(19) United States
(12) Patent Application Publication
(10) Pub. No.: US 2013/0129723 A1
(43) Pub. Date: May 23, 2013

(54) Heterodimer Binding Proteins and Uses Thereof

(71) Applicants: John W. Blankenship, Seattle, WA (US); Philip Tan, Edmonds, WA (US)

(72) Inventors: John W. Blankenship, Seattle, WA (US); Philip Tan, Edmonds, WA (US)

(73) Assignee: Emergent Product Development Seattle, LLC, Seattle, WA (US)

(21) Appl. No.: 13/519,727

(22) PCT Filed: Dec. 29, 2010
(86) PCT No.: PCT/US10/62436

§ 371 Date: Dec. 10, 2012

Related U.S. Application Data
(60) Provisional application No. 61/290,840, filed on Dec. 29, 2009.
(60) Provisional application No. 61/335,266, filed on Jan. 4, 2010.
(60) Provisional application No. 61/366,743, filed on Jul. 22, 2010.

(51) Int. Cl. C07K 16/44

(52) U.S. Cl. CPC C07K 16/44
USPC 424/134.1; 530/387.3; 435/328; 435/69.6

(57) ABSTRACT

The present disclosure provides polypeptide heterodimers formed between two different single chain fusion polypeptides via natural heterodimerization of an immunoglobulin CH1 region and an immunoglobulin light chain constant region (CL). The polypeptide heterodimer comprises two or more binding domains that specifically bind one or more targets (e.g., a receptor). In addition, both chains of the heterodimer further comprise an Fc region portion. The present disclosure also provides nucleic acids, vectors, host cells and methods for making polypeptide heterodimers as well as methods for using such polypeptide heterodimers, such as in directing T cell activation, inhibiting solid malignancy growth, and treating autoimmune or inflammatory conditions.

### CROSS-REFERENCE(S) TO RELATED APPLICATION(S)

This application claims the benefit under 35 U.S.C. §119(e) of U.S. Provisional Patent Application No. 61/290,840, filed Dec. 29, 2009, and U.S. Provisional Patent Application No. 61/365,266, filed Jul. 16, 2010, each of which is herein incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 910180_{—}422PC_SEQUENCE_LISTING.txt. The text file is 839 KB, was created on Dec. 29, 2010, and is being submitted electronically via EFS-Web, concurrent with the filing of the specification.

### BACKGROUND

1. Technical Field

The present disclosure generally provides polypeptide heterodimers, compositions thereof, and methods for making and using such polypeptide heterodimers. More specifically, the polypeptide heterodimers provided herein are formed, in part, via natural heterodimerization between an immunoglobulin CH1 region and an immunoglobulin light chain constant region (CL). In addition, the polypeptide heterodimers provided herein comprise two or more binding domains that specifically bind one or more targets. Furthermore, both single chain polypeptides of the polypeptide heterodimers provided herein each comprise an Fc region portion (e.g., immunoglobulin CH2 and CH3 domains).

2. Description of the Related Art

The process of signal transduction often involves receptor proteins that have extracellular domains, transmembrane domains, and intracellular domains. During ligand binding, cell surface receptor molecules often oligomerize or multimerize (also referred to as “cross-link”) to effectively transmit a signal to the intracellular compartment of the cell. The stimulation or blockade of this interaction between a receptor and a ligand or of the subsequent oligomerization or multimerization of receptors has important therapeutic implications for a wide variety of diseases.

Exemplary molecules useful in modulating receptor and ligand interactions include antibodies or molecules derived from antibodies. For instance, an antibody or its derivative may function as a receptor antagonist that binds to a cell surface receptor and inactivates it by blocking the binding site of an activating ligand or preventing receptor dimerization or multimerization required for activation. In certain other cases, an antibody or its derivative may function as an agonist by binding to and cross-linking multiple membrane receptors, mimicking the function of a natural ligand. Another example is a bispecific antibody derivative that may be used to direct cytotoxic agents or immune effector cells to target sites, such as tumors.

Bispecific antibodies are antibody-based molecules that can simultaneously bind two separate and distinct antigens (or different epitopes of the same antigen). One use of bispecific antibodies has been to redirect cytotoxic immune effector cells for enhanced killing of tumor cells by antibody dependent cellular cytotoxicity (ADCC). In this context, one arm of the bispecific antibody binds an antigen on the tumor cell, and the other binds a determinant expressed on effector cells. By cross-linking tumor and effector cells, the bispecific antibody not only brings the effector cells within the proximity of the tumor cells but also simultaneously triggers their activation, leading to effective tumor cell-killing. Bispecific antibodies have also been used to enrich chemo- or radiotherapeutic agents in tumor tissues to minimize detrimental effects to normal tissue. In this setting, one arm of the bispecific antibody binds an antigen expressed on the cell targeted for destruction, and the other arm delivers a chemotherapeutic drug, radioisotope, or toxin.

A major obstacle in the general development of bispecific antibodies has been the difficulty of producing materials of sufficient quality and quantity for both preclinical and clinical studies. Initially, the main route to the production of bispecific antibodies was by co-expression of both the light chains and both the heavy chains of two parent antibodies of different specificities in a single cell. However, the desired binding-competent bispecific antibodies are a minor product, and purification from the other products is very difficult. Another traditional method for bispecific antibody production is chemical conjugation of two antibodies or their fragments having different specificities. However, this method is also complicated, and the chemical modification process may inactivate the antibody or promote aggregation. Because purification from undesired products remains difficult, the resulting low yield and poor quality of bispecific antibody make this process unsuitable for the large scale production required for clinical development.

Recently, various heterodimerization techniques have been used to improve the production of bispecific antibodies. However, fusion of simple heterodimerization domains like the Jun/Fos coiled-coil to scFv domains lead to a mixture of homo- and heterodimers and need to be assembled by refolding (de Kruif and Logtenberg, J. Biol. Chem. 271: 7630-4, 1996). Fusion of scFv fragments to whole antibodies was also used as a dimerization device (Coloma and Morrison, Nat. Biotechnol. 15:159-63, 1997). However, such fusion results in a large molecule with poor solid tissue penetration capabilities. Fusion of two scFv fragments together has also been used to generate bispecific proteins (e.g., BITE® antibodies by Micromet Inc., Bethesda, Md., U.S. Pat. No. 7,635,472). However, such proteins do not contain Fc regions, and thus do not allow manipulation of their activities via Fc regions. In addition, these proteins are small (˜55 kDa) and thus have relatively short half lives in serum.

To date, immunoglobulin fusion technology has not provided commercially viable heterodimeric proteins or methods for making them. Thus, there remains a need in the art for alternative multispecific heterodimeric proteins as well as efficient methods for producing the same.

### BRIEF SUMMARY

The present disclosure provides polypeptide heterodimers formed between two different single chain polypeptides via natural heterodimerization of an immunoglobulin CH1 region and an immunoglobulin light chain constant region (CL). The present disclosure also provides nucleic acids, vectors, host cells and methods for making polypeptide heterodimers as well as methods for using such polypeptide heterodimers, such as in directed T cell activation, inhibiting solid malignancy growth, treating autoimmune or inflammatory conditions, or treating B-cell associated disorders or diseases.

In one aspect, the present disclosure provides a polypeptide heterodimer that comprises (a) a first single chain polypeptide (SCP-I) comprising from one to four binding domains that specifically bind from one to four targets, a hinge (H-I), an immunoglobulin heterodimerization domain (HD-I), and an Fc region portion (FRP-I); and (b) a second single chain polypeptide (SCP-II) comprising from zero to four binding domains that specifically bind from zero to four targets, a hinge (H-II), an immunoglobulin heterodimerization domain (HD-II), and an Fc region portion (FRP-II); wherein (i) the immunoglobulin HD-I and the immunoglobulin HD-II preferentially associate with each other to form a polypeptide heterodimer comprised of SCP-I and SCP-II, and (1) the immunoglobulin heterodimerization domain of the first single chain polypeptide (HD-I) comprises a first immunoglobulin CH1 region, and the immunoglobulin heterodimerization domain of the second single chain polypeptide (HD-II) comprises a first immunoglobulin CL region, or (2) the immunoglobulin heterodimerization domain of the first single chain polypeptide (HD-I) comprises a first immunoglobulin CL region, and the immunoglobulin heterodimerization domain of the second single chain polypeptide (HD-II) comprises a first immunoglobulin CH1 region; and (ii) the Fc region portion of SCP-I and the Fc region portion of SCP-II comprise an immunoglobulin CH2 and CH3 domain of IgG1, IgG2, IgG3, IgG4, IgA1, IgA2, IgD, or any combination thereof; one or two immunoglobulin CH3 domains of IgG1, IgG2, IgG3, IgG4, IgA1, IgA2, IgD, IgE, IgM, or any combination thereof; or an immunoglobulin CH3 and CH4 domain of IgE, IgM, or any combination thereof, provided that the polypeptide heterodimer comprises at least two binding domains that specifically bind a target, for instance, at least two different targets.

In certain embodiments, the binding domains of the polypeptide heterodimers are single chain Fv (scFv) polypeptides.

In certain embodiments, the polypeptide heterodimer comprises two binding domains (BD1 and BD2). In one embodiment, the two binding domains (BD1 and BD2) are both on the first single chain polypeptide (SCP-I) and wherein the HD-I and FRP-I are disposed between BD1 and BD2. In another embodiment, the first binding domain (BD1) is on the first single chain polypeptide (SCP-I) and the second binding domain (BD2) is on the second single chain polypeptide (SCP-II). For example, the first binding domain (BD1) may be amino terminal to the Fc region portion of the first single chain polypeptide (FRP-I), and the second binding domain (BD2) may amino terminal to the Fc region portion of the second single chain polypeptide (FRP-II). Alternatively, the first binding domain (BD1) may be amino terminal to the Fc region portion of the first single chain polypeptide (FRP-I), and the second binding domain (BD2) may be carboxyl terminal to the Fc region portion of the second single chain polypeptide (FRP-II). Also alternatively, the first binding domain (BD1) may be carboxyl terminal to the Fc region portion of the first single chain polypeptide (FRP-I), and the second binding domain (BD2) may be carboxyl terminal to the Fc region portion of the second single chain polypeptide (FRP-II).

In certain embodiments, the polypeptide heterodimer comprises three binding domains (BD1, BD2 and BD3). In one embodiment, the HD-I and FRP-I are disposed between BD1 and BD2, and the third binding domain (BD3) is amino terminal to the Fc region portion of the second single chain polypeptide (FRP-II). In an alternative embodiment, the HD-I and FRP-I are disposed between BD1 and BD2, and the third binding domain (BD3) is carboxyl terminal to the Fc region portion of the second single chain polypeptide (FRP-II).

In certain embodiments, the polypeptide heterodimer comprises four binding domains (BD1, BD2, BD3, and BD4). For instance, the HD-I and FRP-I may be disposed between BD1 and BD2, and the HD-II and FRP-II may be disposed between BD3 and BD4.

In certain embodiments, the polypeptide heterodimer comprises five to eight binding domains (e.g., 5, 6, 7 or 8 binding domains).

In certain embodiments, at least one of the binding domains of the polypeptide heterodimers provided herein specifically binds to, or is an antagonist of, TCRα, TCRβ, CD3γ, CD3δ, CD3ε, CD28, CD79b, hyperIL-6, monoIL-10, CD86, CD20, PSMA, CD19, HLA-DR, Ron, c-Met, CEACAM-6, LIGHT, GITRL, CD40, PDL1, PDL2, HVEM, LTBR, EGFR, EGFRvIII, ErbB2, ErbB3, ErbB4, IGF1R, EphA2, PDGFR, VEGFR1-4, Angiopoietin 2, CD64, CD32A, CD16, CD71, TNFR1, TNFR2, TWEAKR, TACI, BAFF-R, BCMA, FAS, CD32B, CD21, CD22, CD30, CD33, CD37, CD38, CD70, TNFα, IL-6, hyperIL-6, IL-2, IL-1, IL-7, IL-8, IL-17A/C, IP-10, IFNγ, IFNα, RANKL, FASL, TGFβ, IL10, IL17A/F, CSF2, IGF1, IGF2, BLyS/APRIL, HGF, MSP, EGF (including epiregulin, herregulin, β-regulin, neuregulin), HIF-1α, VEGFA, VEGFB, VEGFC, VEGFD, TNFα, Wnt, sHH, TGFβ, PDGF, TWEAK, EpCAM, CEA, PCTA-1, STEAP-1, PSCA, ALCAM (CD166), EphA2, CD151, CA-125, MUC-1, MAGE-1, TROP2, CCR5, HER-3, HER-4, EGFR, CEA, MUC2, MUC3, MUC4, MUC5_{AC}, MUC5_{b}, MUC7, βhCG, Lewis-Y, ganglioside GD3, 9-O-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRvIII, LG, SAS, CD63, IGF1R, CD151, TGFBR2, GHRHR, GHR, IL-6R, gp130, TNFR2, OSMRβ, Patched-1, Frizzled, Robo1, CD80, CD81, CD86, OX40, CD40, CD137, LIFRβ, TLR7 or TLR9.

In certain other embodiments, at least one of the binding domains of the polypeptide heterodimer is an agonist of IL-10, HLA-G, HGF, IL-35, PD-1, BTLA, TNFR1, TNFR2, DR4, DR5, TWEAKR, or FAS.

In certain polypeptide heterodimers provided herein, at least one binding domain specifically binds a TCR complex or a component thereof, and at least another binding domain specifically binds to PSMA, CD79b, CD19, HLA-DR, CD20, RON, c-Met, or CEACAM-6.

In certain other polypeptide heterodimers provided herein, at least one binding domain specifically binds to CD28, and at least another binding domain specifically binds to, or is an antagonist of, CD79b, hyperIL-6, PDL2, monoIL-10, CD86, LIGHT, GITRL, CD40, PDL1, HVEM, or LTBR.

In certain other polypeptide heterodimers provided herein, at least one binding domain specifically binds to CD28, and at least another binding domain is an agonist of IL-10, HLA-G, HGF, IL-35, PD-1, or BTLA.

In certain embodiments, the immunoglobulin heterodimerization domain of the first single chain polypeptide (HD-I) comprises the first immunoglobulin CH1 region and the immunoglobulin heterodimerization domain of the second single chain polypeptide (HD-II) comprises the first immunoglobulin CL region. In one embodiment, the first CH1 region is amino terminal to the Fc region portion of the first single chain polypeptide, and the first CL region is amino terminal to the Fc region portion of the second single chain polypeptide. In another embodiment, the first CH1 region is carboxyl terminal to the Fc region portion of the first single chain polypeptide, and the first CL region is carboxyl terminal to the Fc region portion of the second single chain polypeptide.

In certain embodiments in which the immunoglobulin heterodimerization domain of the first single chain polypeptide (HD-I) comprises the first immunoglobulin CH1 region and the immunoglobulin heterodimerization domain of the second single chain polypeptide (HD-II) comprises the first immunoglobulin CL region, the first single chain polypeptide further comprises a second CH1 region and the second single chain polypeptide further comprises a second CL region, and the second CH1 region of the first single chain polypeptide and the second CL region of the second single chain polypeptide associate with each other in the polypeptide heterodimer. For instance, the Fc region portion of the first single chain polypeptide may be disposed between the first and second CH1 regions, and the Fc region portion of the second single chain polypeptide may be disposed between the first and second CL regions. Alternatively, both the first and second CH1 regions may be amino terminal to the Fc region portion of the first single chain polypeptide, and both the first and second CL regions may be amino terminal to the Fc region portion of the second single chain polypeptide. Also alternatively, both the first and second CH1 regions may be carboxyl terminal to the Fc region portion of the first single chain polypeptide, and both the first and second CL regions may be carboxyl terminal to the Fc region portion of the second single chain polypeptide.

In certain other embodiments in which the immunoglobulin heterodimerization domain of the first single chain polypeptide (HD-I) comprises the first immunoglobulin CH1 region and the immunoglobulin heterodimerization domain of the second single chain polypeptide (HD-II) comprises the first immunoglobulin CL region, the first single chain polypeptide further comprises a second CL region and the second single chain polypeptide further comprises a second CH1 region, and the second CL region of the first single chain polypeptide and the second CH1 region of the second single chain polypeptide associate with each other in the polypeptide heterodimer. For instance, in one embodiment, in the first single chain polypeptide, the first CH1 region is amino terminal to the Fc region portion and the second CL region is carboxyl terminal to the Fc region portion; and in the second single chain polypeptide, the first CL region is amino terminal to the Fc region portion, and the second CH1 region is carboxyl terminal to the Fc region portion. In another embodiment, in the first single chain polypeptide, the first CH1 region is carboxyl terminal to the Fc region portion, and the second CL region is amino terminal to the Fc region portion; and in the second single chain polypeptide, the first CL region is carboxyl terminal to the Fc region portion, and the second CH1 region is amino terminal to the Fc region portion. In yet another embodiment, in the first single chain polypeptide, both the first CH1 region and the second CL regions are amino terminal to the Fc region portion, and the first CH1 region is amino terminal to the second CL region; and in the second single chain polypeptide, both the first CL region and the second CH1 region are amino terminal to the Fc region portion, and the first CL region is amino terminal to the second CH1 region. In yet another embodiment, in the first single chain polypeptide, both the first CH1 region and the second CL regions are amino terminal to the Fc region portion, and the second CL region is amino terminal to the first CH1 region; and in the second single chain polypeptide, both the first CL region and the second CH1 region are amino terminal to the Fc region portion, and the second CH1 region is amino terminal to the first CL region. In a further embodiment, in the first single chain polypeptide, both the first CH1 region and the second CL regions are carboxyl terminal to the Fc region portion, and the first CH1 region is amino terminal to the second CL region; and in the second single chain polypeptide, both the first CL region and the second CH1 region are carboxyl terminal to the Fc region portion, and the first CL region is amino terminal to the second CH1 region. In another embodiment, in the first single chain polypeptide, both the first CH1 region and the second CL regions are carboxyl terminal to the Fc region portion, and the second CL region is amino terminal to the first CH1 region; and in the second single chain polypeptide, both the first CL region and the second CH1 region are carboxyl terminal to the Fc region portion, and the second CH1 region is amino terminal to the first CL region.

In certain embodiments, the immunoglobulin heterodimerization domain of the first single chain polypeptide (HD-I) comprises a first immunoglobulin CL region, and the immunoglobulin heterodimerization domain of the second single chain polypeptide (HD-II) comprises a first immunoglobulin CH1 region. In one embodiment, the first CL region is amino terminal to the Fc region portion of the first single chain polypeptide, and the first CH1 region is amino terminal to the Fc region portion of the second single chain polypeptide. In another embodiment, the first CL region is carboxyl terminal to the Fc region portion of the first single chain polypeptide, and the first CH1 region is carboxyl terminal to the Fc region portion of the second single chain polypeptide. In yet another embodiment, the first single chain polypeptide further comprises a second CL region and the second single chain polypeptide further comprises a second CH1 region, and the second CL region of the first single chain polypeptide and the second CH1 region of the second single chain polypeptide associate with each other in the polypeptide heterodimer. For instance, the Fc region portion of the first single chain polypeptide may be disposed between the first and second CL regions, and the Fc region portion of the second single chain polypeptide may be disposed between the first and second CH1 regions. Alternatively, both the first and second CL regions may be amino terminal to the Fc region portion of the first single chain polypeptide, and both the first and second CH1 regions may be amino terminal to the Fc region portion of the second single chain polypeptide. Also alternatively, both the first and second CL regions may be carboxyl terminal to the Fc region portion of the first single chain polypeptide, and both the first and second CH1 regions may be carboxyl terminal to the Fc region portion of the second single chain polypeptide.

In certain embodiments, the first CL region is a Cκ region. In other embodiments, the first CL region is a Cλ region.

In certain embodiments, the second CL region is a Cκ region. In other embodiments, the second CL region is a Cλ region.

In certain embodiments, the Cκ region is a wild type human immunoglobulin Cκ region. In certain other embodiments, the Cκ region is an altered human immunoglobulin Cκ region with one or more amino acids of a wild type human Cκ region substituted at N29, N30, Q52, V55, T56, T56, S68, or T70. For example, the one or more amino acid substitutions may be selected from Ala (A), Arg (R), Trp (W), Tyr (Y), Glu (E), Gln (Q), Lys (K), Asp (D), Met (M), Ser (S), and Phe (F).

In certain embodiments, the CH1 region is an altered human immunoglobulin CH1 region comprising an amino acid substitution by which Val (V) at position 68 is substituted by Lys (K), Arg (R) or His (H), and wherein the Ck region is an altered human immunoglobulin Ck region comprising an amino acid substitution by which Leu (L) at position 29 is substituted by Asp (D) or Glu (E). In certain other embodiments, the CH1 region is an altered human immunoglobulin CH1 region comprising an amino acid substitution by which Val (V) at position 68 is changed to Asp (D) or Glu (E), and wherein the Ck region is an altered human immunoglobulin Ck region comprising an amino acid substitution by which Leu (L) at position 29 is changed to Lys (K), Arg (R) or His (H).

In certain embodiments, the Cλ region is a wild type human immunoglobulin Cλ region.

In certain embodiments, the first CH1 region or the second CH1 region when present is a wild type human immunoglobulin CH1 region, such as a wild type human IgG1 CH1 region.

In certain embodiments, the first CH1 region or the second CH1 region when present is an altered human immunoglobulin CH1 region, such as an altered human IgG1 CH1 region, with the cysteine of a wild type human immunoglobulin CH1 region that is involved in forming a disulfide bond with a wild type human immunoglobulin CL region deleted or substituted.

In certain embodiments, the Cκ region is an altered human immunoglobulin Ck region, such as an with the cysteine residue of a wild type human Cκ region that is involved in forming a disulfide bond with a wild type human immunoglobulin CH1 region deleted or substituted.

In certain embodiments, the Cλ region is an altered human immunoglobulin Cλ region with the cysteine residue of a wild type human Cλ region that is involved in forming a disulfide bond with a wild type human immunoglobulin CH1 region deleted or substituted.

In certain embodiments, the first CH1 region and the second CH1 region when present is a polypeptide comprising SEQ ID NO:114, 844 or 845.

In certain embodiments, the Ck region when present is selected from any one of the polypeptides comprising SEQ ID NOS:141-178, 202, and 838-843.

In certain embodiments, the Cλ region when present is a polypeptide comprising SEQ ID NO:140.

In certain embodiments, the Fc region portion of the first single chain polypeptide (FRP-I) and the Fc region portion of the second single chain polypeptide (FRP-II) each comprise an immunoglobulin CH2 domain, such as an IgG1 CH2 domain or an IgG2, IgG3, IgG4, IgA1, IgA2, or IgD CH2 domain.

In certain embodiments, the Fc region portion of the first single chain polypeptide (FRP-I) and the Fc region portion of the second single chain polypeptide (FRP-II) each comprise an immunoglobulin CH3 domain, such as an IgG1 CH3 domain or an IgG2, IgG3, IgG4, IgA1, IgA2, IgD, IgE or IgM CH3 domain.

In certain embodiments, the Fc region portion of the first single chain polypeptide (FRP-I) and the Fc region portion of the second single chain polypeptide (FRP-II) each comprise an immunoglobulin CH2 domain and an immunoglobulin CH3 domain, such as are IgG1, IgG2, IgG3, IgG4, IgA1, IgA2, or IgD CH2 and CH3 domains.

In some embodiments in which the Fc region portion comprises an immunoglobulin CH3 domain that is immediately amino terminal to an immunoglobulin heterodimerization domain (e.g., a CH1 domain or a Ck domain), the immunoglobulin CH3 domain is linked to the CH1 domain immediately carboxyl terminal to the immunoglobulin CH3 domain in one single chain polypeptide via a peptide comprising SEQ ID NOS:846, 847, 848, or 849; and the immunoglobulin CH3 domain is linked to the Ck domain immediately carboxyl terminal to the immunoglobulin CH3 domain in the other single chain polypeptide via a peptide comprising SEQ ID NOs:846, 850, 951, or 852.

In certain embodiments, the Fc region portion of the first single chain polypeptide (FRP-I) and the Fc region portion of the second chain polypeptide (FRP-II) comprise IgM or IgE CH3 and CH4 domains.

In certain embodiments in which the Fc region portion comprises an immunoglobulin CH2 domain, the CH2 domain may be an altered human IgG1, IgG2, IgG3, or IgG4 CH2 domain. Exemplary altered human IgG1, IgG2, IgG3 or IgG4 CH2 domain include those that comprise (a) an amino acid substitution at position 297 and at least one additional substitution or deletion at positions 234 to 238; (b) one or more amino acid mutations at positions 234-238 and at least one substitution at position 253, 310, 318, 320, 322, or 331; or (c) an amino acid substitution at the asparagine of position 297, one or more substitutions or deletions at positions 234 to 238, and at least one substitution at position 253, 310, 318, 320, 322, or 331. Another exemplary CH2 domain is an altered human IgG1 CH2 domain that comprises amino acid substitutions at positions L234, L235, G237, E318, K320 and K322.

In certain embodiments, the CH3 domain of the first single chain polypeptide is an altered human IgG1, IgG2, IgG3, or IgG4 CH3 domain that comprises a T366W, and the CH3 domain of the second single chain polypeptide is an altered human IgG1, IgG2, IgG3 or IgG4 CH3 domain that comprises a Y407A substitution. In certain other embodiments, the CH3 domain of the first single chain polypeptide is an altered human IgG1, IgG2, IgG3, or IgG4 CH3 domain that comprises a T366Y substitution, and the CH3 domain of the second single chain polypeptide is an altered human IgG1, IgG2, IgG3 or IgG4 CH3 domain that comprises a Y407T substitution. In certain other embodiments, the CH3 domain of the first single chain polypeptide is an altered human IgG1, IgG2, IgG3, or IgG4 CH3 domain that comprises a T366W substitution, and the CH3 domain of the second single chain polypeptide is an altered human IgG1, IgG2, IgG3 or IgG4 CH3 domain that comprises T366S, L368A and Y407V substitutions. In certain other embodiments, the CH3 domain of the first single chain polypeptide is an altered human IgG1, IgG2, IgG3, or IgG4 CH3 domain that comprises a Y407A substitution, and the CH3 domain of the second single chain polypeptide is an altered human IgG1, IgG2, IgG3 or IgG4 CH3 domain that comprises a T366W substitution. In certain other embodiments, the CH3 domain of the first single chain polypeptide is an altered human IgG1, IgG2, IgG3, or IgG4 CH3 domain that comprises a Y407T substitution, and the CH3 domain of the second single chain polypeptide is an altered human IgG1, IgG2, IgG3 or IgG4 CH3 domain that comprises a T366Y substitution. In certain other embodiments, the CH3 domain of the first single chain polypeptide is an altered human IgG1, IgG2, IgG3, or IgG4 CH3 domain that comprises T366S, L368A and Y407W substitutions, and the CH3 domain of the second single chain polypeptide is an altered human IgG1, IgG2, IgG3 or IgG4 CH3 domain that comprises a T366W substitution.

In certain embodiments, the hinge of both the first and second single chain polypeptides is an immunoglobulin hinge region, such as an IgG1, IgG2, IgG3, IgG4, IgA1, IgA2, IgD, or IgE hinge. In certain embodiments, the immunoglobulin hinge is a wild type immunoglobulin hinge. In certain other embodiments, the immunoglobulin hinge is an altered immunoglobulin hinge selected from SEQ ID NOS:232, 234, 240, 664-673, 675 and 676.

In certain embodiments, the hinge region is (a) at the amino terminal to the Fc region portion, (b) disposed between the binding domain and the immunoglobulin heterodimerization domain, (c) disposed between the immunoglobulin heterodimerization domain and the Fc region portion, (d) at the amino terminus of the first or second single chain polypeptide, (e) disposed between the Fc region portion and a binding domain, or (f) at the carboxyl terminus of the first or second single chain polypeptide.

In certain embodiments, at least one of the first and second single chain polypeptide hinges is a C-type lectin hinge region, such as an NKg2A or NKg2D peptide, or a derivative thereof.

In certain embodiments, the hinges of the first and second single chain polypeptides are identical. In certain other embodiments, the hinges of the first and second single chain polypeptides are different.

In certain embodiments, the first single chain polypeptide comprises a binding domain that specifically binds a TCR complex or a component thereof, and the second single chain polypeptide comprises a binding domain that specifically binds CD19, CD79b, HLA-DR or CD20.

In certain embodiments, the first single chain polypeptide comprises a binding domain that specifically binds CD28, and the second single chain polypeptide comprises a binding domain (a) that specifically binds CD79b, hyperIL-6, or CD86 or (b) that comprises a PDL ectodomain or a monoIL-10.

In certain embodiments, the first single chain polypeptide comprises a binding domain that specifically binds c-Met, and the second single chain polypeptide comprises a binding domain that specifically binds RON.

In certain embodiments, the first and second single chain polypeptides comprise SEQ ID NOS: SEQ ID NOS:2 and 4, SEQ ID NOS:6 and 8, SEQ ID NOS:10 and 12, SEQ ID NOS:14 and 16, SEQ ID NOS:18 and 20, SEQ ID NOS:20 and 22, SEQ ID NOS:20 and 24, SEQ ID NOS:30 and 32, SEQ ID NOS:29 and 31, SEQ ID NOS:29 and 32, SEQ ID NOS:30 and 72, SEQ ID NOS:53 and 72, SEQ ID NOS:54 and 72, SEQ ID NOS:55 and 72, SEQ ID NOS:70 and 72, SEQ ID NOS:71 and 72, SEQ ID NOS:63 and 56, SEQ ID NOS:64 and 57, SEQ ID NOS:65 and 60, SEQ ID NOS:66 and 58, SEQ ID NOS:67 and 59, SEQ ID NOS:68 and 61, SEQ ID NOS:69 and 62, SEQ ID NOS:54 and 811, SEQ ID NOS:54 and 812, SEQ ID NOS:54 and 813, SEQ ID NOS:814 and 818, SEQ ID NOS:815 and 818, SEQ ID NOS:816 and 818, SEQ ID NOS:817 and 818, SEQ ID NOS:814 and 820, SEQ ID NOS:814 and 821, SEQ ID NOS:54 and 819, SEQ ID NOS:814 and 826, SEQ ID NOS:814 and 822, SEQ ID NOS:814 and 823, SEQ ID NOS:814 and 824, SEQ ID NOS:859 and 862, SEQ ID NOS:860 and 863, SEQ ID NOS:861 and 864, SEQ ID NOS:874 and 825, SEQ ID NOS:875 and 879, SEQ ID NOS:876 and 880, SEQ ID NOS:877 and 881, or SEQ ID NOS:878 and 882.

In another aspect, the present disclosure provides a composition comprising a polypeptide heterodimer provided herein and a pharmaceutically acceptable excipient.

In another aspect, the present disclosure provides an expression vector capable of expressing a polypeptide heterodimer provided herein, comprising a first polynucleotide encoding the first single chain polypeptide and a second polynucleotide encoding the second single chain polypeptide.

In another aspect, the present disclosure provides a host cell comprising the above expression vector.

In another aspect, the present disclosure provides a host cell comprising first and second expression vectors capable of expressing the first and second single chain polypeptides, respectively, of the polypeptide heterodimer provided herein.

In another aspect, the present disclosure provides a method for making a polypeptide heterodimer, comprising (a) culturing a host cell provided herein under conditions suitable to express first and second single chain polypeptides, and (b) optionally isolating or purifying the heterodimers formed from the first and second single chain polypeptides from the culture.

In another aspect, the present disclosure provides a method for directing T cell activation, comprising administering to a patient in need thereof an effective amount of a polypeptide heterodimer that comprises a binding domain that specifically binds TCRα, TCRβ, CD3γ, CD3δ, CD3ε, or a combination thereof, and a second binding domain that specifically binds a different target, for instance, a tumor-specific antigen or other antigen of choice at a site or cell where T cell activation is desired.

In another aspect, the present disclosure provides a method for inhibiting growth, metastatis, or metastatic growth of a malignancy, comprising administering to a patient in need thereof an effective amount of a polypeptide heterodimer that comprises a binding domain that specifically binds TCRα, TCRβ, CD3γ, CD3δ, CD3ε, c-Met, RON, or a combination thereof. In certain embodiments, the method further comprises administering to a patient in need thereof a chemotherapeutic agent or ionizing radiation.

In another aspect, the present disclosure provides a method for treating an autoimmune or inflammatory condition, comprising administering to a patient in need thereof an effective amount of a polypeptide heterodimer that comprises a binding domain that specifically binds TCRα, TCRβ, CD3γ, CD3δ, CD3ε, or CD28.

In another aspect, the present disclosure provides a method for treating a B-cell associated disorder or disease comprising administering to a patient in need thereof an effective amount of a polypeptide heterodimer that comprises a binding domain that specifically binds TCRα, TCRβ, CD3γ, CD3δ, or CD3ε, and a second binding domain that specifically binds to CD19, CD20, CD79b or HLA-DR.

In certain embodiments, the methods for using the polypeptide heterodimers provided herein may further comprise administering to a patient in need thereof a second active agent, such as a second polypeptide heterodimer, a monoclonal antibody, or an immunoglobulin-derived fusion protein.

### BRIEF DESCRIPTION OF THE DRAWINGS

FIG. 1 is a schematic representation of bivalent anti-CD28 polypeptide heterodimer X0172 (left) and SDS-PAGE analysis of X0172 (right). “NR” stands for “Non-Reduced,” and “Red” stands for “Reduced.”

FIG. 2 shows that both a monovalent (X0124) and a bivalent (X0172) anti-CD28 polypeptide heterodimer synergize with a suboptimal concentration of PMA in stimulating purified human T cells when compared to an anti-CD28 scFv (2E12 scFv) but less than a bivalent anti-CD28 SMIP protein (2E12 SMIP).

FIG. 3 shows that bivalent polypeptide heterodimer X0172 binds to CD4 T cells better than 2E12 scFv and monovalent polypeptide heterodimer X0124.

FIG. 4 shows cation exchange chromatography of polypeptide heterodimers X0251, X0252 and X0253 (left) and SDS-PAGE electrophoresis analysis of the same polypeptide heterodimers under non-reduced (NR) and reduced (Red) conditions (right).

FIG. 5 shows the mass spectra of polypeptide heterodimer X0252, which demonstrates that the heterodimer is the predominant species.

FIG. 6 shows SDS-PAGE electrophoresis analysis of polypeptide heterodimers X0283 and X0284 under non-reduced (NR) and reduced (Red) conditions (left) and cation exchange chromatography analysis of polypeptide heterodimer X0283.

FIG. 7 shows direct binding to CD86 by polypeptide heterodimer X0283 as examined by BIACORE® analysis, with response units (Ru) plotted against time (left) and a schematic representation of X0283.

FIGS. 8A and 8B show binding of bispecific anti-RON and anti-CD3 constructs (polypeptide heterodimer S0268 and Scorpion protein S0266) to MDA-MB-453 cells (A) and to isolated T cells (B).

FIGS. 9A and 9B show specificity of binding to (A) Rec1 (CD19⁺, CD20⁺) cells or (B) Jurkat (CD3⁺) cells by bispecific heterodimers having either anti-CD19 and anti-CD3 binding domains (TSC020) or having anti-CD20 and anti-CD3 binding domains (TSC021).

FIGS. 10A-10D show proliferation of CD4+ and CD8+ T-cells in response to bispecific polypeptide heterodimers TSC054, TSC078, TSC079, and bsc19×3 (TSC036) with (A and B) Daudi (CD19⁺) cells or (C and D) MDA-MB-453 (CD19⁻) cells.

FIGS. 11A and 11B shows T-cell directed cytotoxicity induced by bispecific polypeptide heterodimers TSC054, TSC078, TSC079, and S0268 in a chromium (⁵¹Cr) release assay with (A) Daudi (RON⁻, CD19⁺) cells or (B) BxPC-3 (RON⁺, CD19⁻) cells.

FIG. 12 shows target-dependent T-cell proliferation induced by a CD19+ cell line (Daudi) using bispecific polypeptide heterodimers TSC165, TSC166, TSC167, TSC168 and TSC100 at concentrations from 0.1 pM to 10000 pM.

FIG. 13 shows target-dependent T-cell proliferation induced by a CD19+ cell line (Daudi) using bispecific polypeptide heterodimers TSC127 and TS165 with bsc19×3 BiTE as a control at concentrations from 0.001 pM to 1000 pM.

FIG. 14 shows target-dependent redirected T-cell cytotoxicity on a CD19+ cell line (Daudi) using bispecific polypeptide heterodimers TSC100, TSC165, TC166, TSC167, and TSC168.

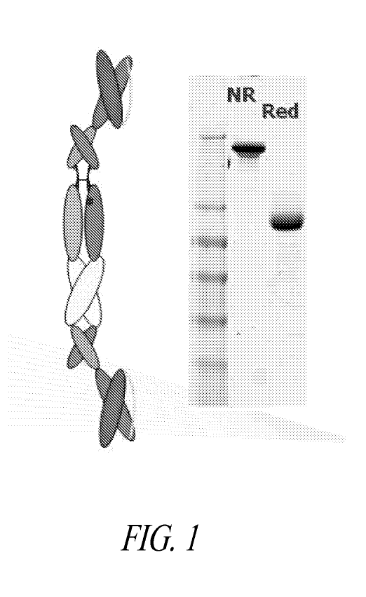

### DETAILED DESCRIPTION

The present disclosure provides polypeptide heterodimers formed between two different single chain polypeptides via natural heterodimerization of an immunoglobulin CH1 region and an immunoglobulin light chain constant region (CL). The polypeptide heterodimer has two or more binding domains that specifically bind one or more targets (e.g., an antigen, a receptor, or a ligand). In addition, both chains of a heterodimer each further comprise an Fc region portion (e.g., immunoglobulin CH2 and/or CH3 domains). The present disclosure also provides nucleic acids, vectors, host cells and methods for making polypeptide heterodimers.

The heterodimerization technology described herein has one or more of the following advantages: (1) potential for minimal immunogenicity of the polypeptide heterodimers because the dimers are formed via natural heterodimerization of an immunoglobulin CH1 region and an immunoglobulin CL region; (2) efficient production and purification of polypeptide heterodimers of the present disclosure is possible by co-expressing the two different single chain polypeptides, as shown in the examples; (3) the ability to mediate Fc effector functions (e.g., CDC, ADCC, ADCP), which can be modulated up or down by mutagenesis, and a longer serum half life because each chain of a polypeptide heterodimer according to the present disclosure has an Fc region portion (e.g., immunoglobulin CH2 and CH3 domains); and (4) polypeptide heterodimers of the present disclosure have a size that is typically smaller than an antibody molecule, which can allow for better tissue penetration, such as into a solid malignancy.

The polypeptide heterodimers provided herein are useful to direct therapeutic agents or immune effector cells to target cells. For instance, in certain embodiments, the polypeptide heterodimers may comprise a binding domain that specifically binds a TCR complex or a component thereof (e.g., TCRα, TCRβ, CD3γ, CD3δ, and CD3ε) and another binding domain that specifically binds a second different target, such as an oncology target (e.g., c-Met, RON, CEACAM-6, and PSMA) or a B-cell target (e.g., CD19, CD79b, HLA-DR and CD20). The binding domain specific for the second different target may have a higher affinity for its target than the affinity of the binding domain for the TCR complex or component thereof, such as CD3. Such polypeptide heterodimers will preferably bind to the oncology target or the B-cell target first and subsequently recruit T cells to the tumor or cancer cells expressing the oncology target or B-cell target and are useful in inhibiting growth, metastasis or metastatic growth of a malignancy (including B-cell cancers). Additional uses of polypeptide heterodimers provided herein include directed T cell activation and treating autoimmune or inflammatory conditions.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts that term's definition in the application, the definition that appears in this application controls.

In the present description, any concentration range, percentage range, ratio range, or integer range is to be understood to include the value of any integer within the recited range and, when appropriate, fractions thereof (such as one tenth and one hundredth of an integer), unless otherwise indicated. As used herein, “about” means±20% of the indicated range, value, sequence, or structure, unless otherwise indicated. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives. As used herein, the terms “include” and “comprise” are used synonymously. In addition, it should be understood that the individual single chain polypeptides or heterodimers derived from various combinations of the structures and substituents (e.g., domains, regions, hinges, and linkers) described herein are disclosed by the present application to the same extent as if each single chain polypeptide or heterodimer were set forth individually. Thus, selection of particular components to form individual single chain polypeptides or heterodimers is within the scope of the present disclosure.

As used herein, a protein “consists essentially of” several domains (e.g., a binding domain that specifically binds a target, a hinge, an immunoglobulin heterodimerization domain, and an Fc region portion) if the other portions of the protein (e.g., amino acids at the amino- or carboxy-terminus or between two domains), in combination, contribute to at most 20% (e.g., at most 15%, 10%, 8%, 6%, 5%, 4%, 3%, 2% or 1%) of the length of the protein and do not substantially affect (i.e., do not reduce the activity by more than 50%, such as more than 40%, 30%, 25%, 20%, 15%, 10%, or 5%) the activities of various domains (e.g., the target binding affinity of the binding domain, the activities of the Fc region portion, and the capability of the immunoglobulin heterodimerization domain in facilitating heterodimerization). In certain embodiments, a protein (e.g., a single chain polypeptide) consists essentially of a binding domain that specifically binds a target, an immunoglobulin heterodimerization domain, a hinge, and an Fc region portion may comprise junction amino acids at the amino- and/or carboxy-terminus of the protein and/or between two different domains (e.g., between the binding domain and the immunoglobulin heterodimerization domain, between the immunoglobulin heterodimerization domain and the hinge, and/or between the hinge and the Fc region portion).

A “polypeptide heterodimer” or “heterodimer,” as used herein, refers to a dimer formed from two different single chain polypeptides. This term does not include an antibody formed from four single chain polypeptides (i.e., two light chains and two heavy chains). A “dimer” refers to a biological entity that consists of two subunits associated with each other via one or more forms of intramolecular forces, including covalent bonds (e.g., disulfide bonds) and other interactions (e.g., electrostatic interactions, salt bridges, hydrogen bonding, and hydrophobic interactions), and is stable under appropriate conditions (e.g., under physiological conditions, in an aqueous solution suitable for expressing, purifying, and/or storing recombinant proteins, or under conditions for non-denaturing and/or non-reducing electrophoresis).

A “single chain polypeptide” is a single, linear and contiguous arrangement of covalently linked amino acids. It does not include two polypeptide chains that link together in a non-linear fashion, such as via an interchain disulfide bond (e.g., a half immunoglobulin molecule in which a light chain links with a heavy chain via a disulfide bond). In certain embodiments, a single chain polypeptide may have or form one or more intrachain disulfide bonds.

An “immunoglobulin heterodimerization domain,” as used herein, refers to an immunoglobulin domain of a single chain polypeptide that preferentially interacts or associates with a different immunoglobulin domain of another single chain polypeptide wherein the interaction of the different heterodimerization domains substantially contributes to or efficiently promotes heterodimerization of the first and second single chain polypeptides (i.e., the formation of a dimer between two different single chain polypeptides, which is also referred to as a “heterodimer”). The interaction(s) between heterodimerization domains “substantially contributes to or efficiently promotes” the heterodimerization of first and second single chain polypeptides if there is a statistically significant reduction in the dimerization between the first and second single chain polypeptides in the absence of the heterodimerization domain of the first single chain polypeptide (HD-I) and/or the heterodimerization domain of the second single chain polypeptide (HD-II). In certain embodiments, when the first and second single chain polypeptides are co-expressed, at least 60%, at least about 60% to about 70%, at least about 70% to about 80%, at least about 80% to about 90%, 91%, 92%, 93%, 94%, 95%, 96%, 97%, 98%, 99%, or 100%, and at least about 90% to about 92%, 93%, 94%, 95%, 96%, 97%, 98%, or 99% of the first and second single chain polypeptides form heterodimers with each other. Representative immunoglobulin heterodimerization domains of the present disclosure include an immunoglobulin CH1 region, an immunoglobulin CL region (e.g., Cκ or Cλ isotypes), or derivatives thereof, including wild type immunoglobulin CH1 and CL regions and altered (or mutated) immunoglobulin CH1 and CL regions, as provided herein.

A “binding domain” or “binding region,” as used herein, refers to a protein, polypeptide, oligopeptide, or peptide that possesses the ability to specifically recognize and bind to a target (e.g., CD3, TCR, CD28, c-Met, RON). A binding domain includes any naturally occurring, synthetic, semi-synthetic, or recombinantly produced binding partner for a biological molecule or another target of interest. Exemplary binding domains include single chain antibody variable regions (e.g., domain antibodies, sFv, scFv, Fab), receptor ectodomains (e.g., c-Met, RON), or ligands (e.g., cytokines, chemokines). A variety of assays are known for identifying binding domains of the present disclosure that specifically bind a particular target, including Western blot, ELISA, and Biacore analysis.

A binding domain and a fusion protein thereof “specifically binds” a target if it binds the target with an affinity or Ka (i.e., an equilibrium association constant of a particular binding interaction with units of 1/M) equal to or greater than 10⁵ M⁻¹, while not significantly binding other components present in a test sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low affinity” binding domains refer to those binding domains with a Kₐ of up to 10⁷ M⁻¹, up to 10⁶ M⁻¹, up to 10⁵ M⁻¹. Alternatively, affinity may be defined as an equilibrium dissociation constant (K_{d}) of a particular binding interaction with units of M (e.g., 10⁻⁵ M to 10⁻¹³ M). Affinities of binding domain polypeptides and single chain polyepeptides according to the present disclosure can be readily determined using conventional techniques (see, e.g., Scatchard et al. (1949) Ann. N.Y. Acad. Sci. 51:660; and U.S. Pat. Nos. 5,283,173, 5,468,614, or the equivalent).

“T cell receptor” (TCR) is a molecule found on the surface of T cells that, along with CD3, is generally responsible for recognizing antigens bound to major histocompatibility complex (MHC) molecules. It consists of a disulfide-linked heterodimer of the highly variable α and β chains in most T cells. In other T cells, an alternative receptor made up of variable γ and δ chains is expressed. Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see, Abbas and Lichtman, Cellular and Molecular Immunology (5th Ed.), Editor: Saunders, Philadelphia, 2003; Janeway et al., Immunobiology: The Immune System in Health and Disease, 4^{th} Ed., Current Biology Publications, p 148, 149, and 172, 1999). TCR as used in the present disclosure may be from various animal species, including human, mouse, rat, or other mammals.

“CD3” is known in the art as a multi-protein complex of six chains (see, Abbas and Lichtman, 2003; Janeway et al., p. 172 and 178, 1999). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. It is believed the ITAMs are important for the signaling capacity of a TCR complex. CD3 as used in the present disclosure may be from various animal species, including human, mouse, rat, or other mammals.

“TCR complex,” as used herein, refers to a complex formed by the association of CD3 with TCR. For example, a TCR complex can be composed of a CD3γ chain, a CD3δ chain, two CD3ε chains, a homodimer of CD3ζ chains, a TCRα chain, and a TCRβ chain. Alternatively, a TCR complex can be composed of a CD3γ chain, a CD3δ chain, two CD3ε chains, a homodimer of CD3ζ chains, a TCRγ chain, and a TCRδ chain.

“A component of a TCR complex,” as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

Terms understood by those in the art of antibody technology are each given the meaning acquired in the art, unless expressly defined differently herein. Antibodies are known to have variable regions, a hinge region, and constant domains. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

For example, the terms “VL” and “VH” refer to the variable binding region from an antibody light and heavy chain, respectively. The variable binding regions are made up of discrete, well-defined sub-regions known as “complementarity determining regions” (CDRs) and “framework regions” (FRs). The term “CL” refers to an “immunoglobulin light chain constant region” or a “light chain constant region,” i.e., a constant region from an antibody light heavy chain. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM). A “Fab” (fragment antigen binding) is the part of an antibody that binds to antigens and includes the variable region and CH1 of the heavy chain linked to the light chain via an inter-chain disulfide bond.

As used herein, “an Fc region constant domain portion” or “Fc region portion” refers to the heavy chain constant region segment of the Fc fragment (the “fragment crystallizable” region or Fc region) from an antibody, which can include one or more constant domains, such as CH2, CH3, CH4, or any combination thereof. By way of background, the Fc region is responsible for the effector functions of an immunoglobulin, such as ADCC (antibody-dependent cell-mediated cytotoxicity), ADCP (antibody-dependent cellular phagocytosis), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al., Nature, 337:525 (1989)).

In addition, antibodies have a hinge sequence that is typically situated between the Fab and Fc region (but a lower section of the hinge may include an amino-terminal portion of the Fc region). By way of background, an immunoglobulin hinge acts as a flexible spacer to allow the Fab portion to move freely in space. In contrast to the constant regions, hinges are structurally diverse, varying in both sequence and length between immunoglobulin classes and even among subclasses. For example, a human IgG1 hinge region is freely flexible, which allows the Fab fragments to rotate about their axes of symmetry and move within a sphere centered at the first of two inter-heavy chain disulfide bridges. By comparison, a human IgG2 hinge is relatively short and contains a rigid poly-proline double helix stabilized by four inter-heavy chain disulfide bridges, which restricts the flexibility. A human IgG3 hinge differs from the other subclasses by its unique extended hinge region (about four times as long as the IgG1 hinge), containing 62 amino acids (including 21 prolines and 11 cysteines), forming an inflexible poly-proline double helix and providing greater flexibility because the Fab fragments are relatively far away from the Fc fragment. A human IgG4 hinge is shorter than IgG1 but has the same length as IgG2, and its flexibility is intermediate between that of IgG1 and IgG2.

According to crystallographic studies, an IgG hinge domain can be functionally and structurally subdivided into three regions: the upper, the core or middle, and the lower hinge regions (Shin et al., Immunological Reviews 130:87 (1992)). Exemplary upper hinge regions include EPKSCDKTHT (SEQ ID NO:227) as found in IgG1, ERKCCVE (SEQ ID NO:211) as found in IgG2, ELKTPLGDTT HT (SEQ ID NO:245) or EPKSCDTPPP (SEQ ID NO:246) as found in IgG3, and ESKYGPP (SEQ ID NO:247) as found in IgG4. Exemplary middle or core hinge regions include CPPCP (SEQ ID NO:228) as found in IgG1 and IgG2, CPRCP (SEQ ID NO:248) as found in IgG3, and CPSCP (SEQ ID NO:249) as found in IgG4. While IgG1, IgG2, and IgG4 antibodies each appear to have a single upper and middle hinge, IgG3 has four in tandem—one being ELKTPLGDTTHTCPRCP (SEQ ID NO:250) and three being EPKSCDTPPP CPRCP (SEQ ID NO:251).

IgA and IgD antibodies appear to lack an IgG-like core region, and IgD appears to have two upper hinge regions in tandem (see SEQ ID NOS:222 and 252). Exemplary wild type upper hinge regions found in IgA1 and IgA2 antibodies are set forth in SEQ ID NOS:215 and 216.

IgE and IgM antibodies, in contrast, lack a typical hinge region and instead have a CH2 domain with hinge-like properties. Exemplary wild-type CH2 upper hinge-like sequences of IgE and IgM are set forth in SEQ ID NO:253 (VCSRDFTPPTVKILQSSSDGGGHFPPTIQLLCLVSGYTPGTINITWLEDG QVMDVDLSTASTTQEGELASTQSELTLSQKHWLSDRTYTCQVTYQGHTFE DSTKKCA) and SEQ ID NO:254 (VIAELPPKVSVFVPPRDGFFGNPRKSKLIC QATGFSPRQIQVSWLREGKQVGSGVTTDQVQAEAKESGPTTYKVTSTLTI KESDWLGQSMFTCRVDHRGLTFQQNASSMCVP), respectively.

As used herein, a “hinge region” or a “hinge” refers to (a) an immunoglobulin hinge region (made up of, for example, upper and core regions) or a functional variant thereof, including wild type and altered immunoglobulin hinges, (b) a lectin interdomain region or a functional variant thereof, (c) a cluster of differentiation (CD) molecule stalk region or a functional variant thereof, or (d) a portion of a cell surface receptor (interdomain region) that connects immunoglobulin V-like or immunoglobulin C-like domains.

As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody. In certain embodiments, a wild type immunoglobulin hinge region sequence is human, and may comprises a human IgG hinge region. Exemplary human wild type immunoglobulin hinge regions are set forth in SEQ ID NOS:215 (IgA1 hinge), 216 (IgA2 hinge), 217 (IgD hinge), 667 (IgG1 hinge), 219 (IgG2 hinge), 220 (IgG3 hinge) and 221 (IgG4 hinge).

An “altered wild type immunoglobulin hinge region” or “altered immunoglobulin hinge region” refers to (a) a wild type immunoglobulin hinge region with up to 30% amino acid changes (e.g., up to 25%, 20%, 15%, 10%, or 5% amino acid substitutions or deletions), or (b) a portion of a wild type immunoglobulin hinge region that has a length of about 5 amino acids (e.g., about 5, 6, 7, 8, 9, 10, 11, 12, 13, 14, 15, 16, 17, 18, 19, or 20 amino acids) up to about 120 amino acids (for instance, having a length of about 10 to about 40 amino acids or about 15 to about 30 amino acids or about 15 to about 20 amino acids or about 20 to about 25 amino acids), has up to about 30% amino acid changes (e.g., up to about 25%, 20%, 15%, 10%, 5%, 4%, 3%, 2%, or 1% amino acid substitutions or deletions or a combination thereof), and has an IgG core hinge region as set forth in SEQ ID NOS:228, 248, or 249.

A “peptide linker” refers to an amino acid sequence that connects a heavy chain variable region to a light chain variable region and provides a spacer function compatible with interaction of the two sub-binding domains so that the resulting polypeptide retains a specific binding affinity to the same target molecule as an antibody that comprises the same light and heavy chain variable regions. In certain embodiments, a linker is comprised of five to about 35 amino acids, for instance, about 15 to about 25 amino acids.

“Junction amino acids” or “junction amino acid residues” refer to one or more (e.g., about 2-10) amino acid residues between two adjacent regions or domains of a single chain polypeptide, such as between a hinge and an adjacent Fc region portion or between a hinge and an adjacent binding domain or between a peptide linker that links two immunoglobulin variable domains and an adjacent immunoglobulin variable domain. Junction amino acids may result from the construct design of a single chain polypeptide (e.g., amino acid residues resulting from the use of a restriction enzyme site during the construction of a nucleic acid molecule encoding a single chain polypeptide).

A “linker between CH3 and CH1 or CL” refers to one or more (e.g., about 2-12) amino acid residues between the C-terminus of a CH3 domain (e.g., a wild type CH3 or a mutated CH3) and the N-terminus of a CH1 domain or CL domain (e.g., Ck).

A “wild type immunoglobulin region” or “wild type immunoglobulin domain” refers to a naturally occurring immunoglobulin region or domain (e.g., a naturally occurring VL, VH, hinge, CL, CH1, CH2, CH3, or CH4) from various immunoglobulin classes or subclasses (including, for example, IgG1, IgG2, IgG3, IgG4, IgA1, IgA2, IgD, IgE, and IgM) and from various species (including, for example, human, sheep, mouse, rat, and other mammals). Exemplary wild type human CH1 regions are set forth in SEQ ID NOS:114, 186-192 and 194, wild type human Cκ region in SEQ ID NO:112, wild type human Cλ regions in SEQ ID NO:113 and 224-226, wild type human CH2 domains in SEQ ID NOS:115, 195-201 and 203, wild type human CH3 domains in SEQ ID NOS:116, 204-210 and 212, and wild type human CH4 domains in SEQ ID NO:213 and 214.

An “altered immunoglobulin region,” “altered immunoglobulin domain,” “mutated immunoglobulin domain,” or the like, refers to an immunoglobulin region with a sequence identity to a wild type immunoglobulin region or domain (e.g., a wild type VL, VH, hinge, CL, CH1, CH2, CH3, or CH4) of at least 75% (e.g., 80%, 82%, 84%, 86%, 88%, 90%, 91%, 92%, 93%, 94%, 95%, 96%, 97%, 98%, 99%, or 99.5%). For example, an “altered immunoglobulin CH1 region” or “altered CH1 region” refers to a CH1 region with a sequence identity to a wild type immunoglobulin CH1 region (e.g., a human CH1) of at least 75% (e.g., 80%, 82%, 84%, 86%, 88%, 90%, 91%, 92%, 93%, 94%, 95%, 96%, 97%, 98%, 99%, or 99.5%). Similarly, an “altered immunoglobulin CH2 domain” or “altered CH2 domain” refers to a CH2 domain with a sequence identity to a wild type immunoglobulin CH2 region (e.g., a human CH2) of at least 75% (e.g., 80%, 82%, 84%, 86%, 88%, 90%, 91%, 92%, 93%, 94%, 95%, 96%, 97%, 98%, 99%, or 99.5%).

“Sequence identity,” as used herein, refers to the percentage of amino acid residues in one sequence that are identical with the amino acid residues in another reference polypeptide sequence after aligning the sequences and introducing gaps, if necessary, to achieve the maximum percent sequence identity, and not considering any conservative substitutions as part of the sequence identity. The percentage sequence identity values are generated by the NCBI BLAST2.0 software as defined by Altschul et al. (1997) “Gapped BLAST and PSI-BLAST: a new generation of protein database search programs,” Nucleic Acids Res. 25:3389-3402, with the parameters set to default values.

In certain embodiments, an altered immunoglobulin domain only contains conservative amino acid substitutions of a wild type immunoglobulin domain. In certain other embodiments, an altered immunoglobulin domain only contains non-conservative amino acid substitutions of a wild type immunoglobulin domain. In yet other embodiments, an altered immunoglobulin domain contains both conservative and non-conservative amino acid substitutions.

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:N.Y. (1975), pp. 71-77; Lewin, Genes IV, Oxford University Press, NY and Cell Press, Cambridge, Mass. (1990), p. 8). In certain embodiments, a conservative substitution includes a leucine to serine substitution.

As used herein, the term “derivative” refers to a modification of one or more amino acid residues of a peptide by chemical or biological means, either with or without an enzyme, e.g., by glycosylation, alkylation, acylation, ester formation, or amide formation. Generally, a “derivative” differs from an “analogue” in that a parent polypeptide may be the starting material to generate a “derivative,” whereas the parent polypeptide may not necessarily be used as the starting material to generate an “analogue.” A derivative may have different chemical, biological or physical properties of the parent polypeptide. For example, a derivative may be more hydrophilic or it may have altered reactivity (e.g., a CDR having an amino acid change that alters its affinity for a target) as compared to the parent polypeptide.

As used herein, unless otherwise provided, a position of an amino acid residue in a variable region of an immunoglobulin molecule is numbered according to the Kabat numbering convention (Kabat, Sequences of Proteins of Immunological Interest, 5^{th} ed. Bethesda, Md.: Public Health Service, National Institutes of Health (1991)), and a position of an amino acid residue in a constant region of an immunoglobulin molecule is numbered according to EU nomenclature (Ward et al., 1995 Therap. Immunol. 2:77-94).

A “receptor” is a protein molecule present in the plasma membrane or in the cytoplasm of a cell to which a signal molecule (i.e., a ligand, such as a hormone, a neurotransmitter, a toxin, a cytokine) may attach. The binding of the signal molecule to the receptor results in a conformational change of the receptor, which ordinarily initiates a cellular response. However, some ligands merely block receptors without inducing any response (e.g., antagonists). Some receptor proteins are peripheral membrane proteins. Many hormone and neurotransmitter receptors are transmembrane proteins that are embedded in the phospholipid bilayer of cell membranes, and another major class of receptors are intracellular proteins such as those for steroid and intracrine peptide hormone receptors.

“B-cell associated disorder or disease” or “a disease or disorder associated with aberrant B-cell activity” refers to a disease or disorder associated with (e.g., causing or resulting from) aberrant B-cell activity or activity that deviates from the normal, proper, or expected course. For example, a B-cell associated disorder or disease may include inappropriate proliferation of B-cells that have damaged or defective DNA or other cellular components. Aberrant B-cell activity may include cell proliferation characterized by inappropriately high levels of B-cell division, inappropriately low levels of B-cell apoptosis, or both. Such diseases may have, for example, single or multiple local abnormal proliferations of B-cells, groups of B-cells or tissue(s), whether cancerous or non-cancerous, benign or malignant. A B-cell associated disorder or disease may also include aberrant antibody production, such as production of autoantibodies, or overproduction of antibodies more desirable when produced at normal levels. It is also contemplated herein that aberrant B-cell activity may occur in certain subpopulations of B-cells and not in other subpopulations, or may include inappropriate stimulation of T-cells, such as by inappropriate antigen presentation to T-cells or by other B-cell pathways. Examples of B-cell associated disorders or diseases include a B-cell malignancy or B-cell cancer (e.g., B-cell lymphoma, B-cell leukemia or B-cell myeloma), a disease characterized by autoantibody production (e.g., autoimmune diseases) or inflammation or a disease characterized by inappropriate T-cell stimulation caused by inappropriate B-cell antigen presentation to T-cells or caused by other pathways involving B-cells.

“Treatment,” “treating” or “ameliorating” refers to either a therapeutic treatment or prophylactic/preventative treatment. A treatment is therapeutic if at least one symptom of disease in an individual receiving treatment improves or a treatment may delay worsening of a progressive disease in an individual, or prevent onset of additional associated diseases.

A “therapeutically effective amount (or dose)” or “effective amount (or dose)” of a specific binding molecule or compound refers to that amount of the compound sufficient to result in amelioration of one or more symptoms of the disease being treated in a statistically significant manner. When referring to an individual active ingredient, administered alone, a therapeutically effective dose refers to that ingredient alone. When referring to a combination, a therapeutically effective dose refers to combined amounts of the active ingredients that result in the therapeutic effect, whether administered serially or simultaneously (in the same formulation or concurrently in separate formulations).

The term “pharmaceutically acceptable” refers to molecular entities and compositions that do not produce allergic or other serious adverse reactions when administered using routes well known in the art.

A “patient in need” refers to a patient at risk of, or suffering from, a disease, disorder or condition that is amenable to treatment or amelioration with a polypeptide heterodimer or a composition thereof provided herein.

The term “immunoglobulin-derived fusion protein,” as used herein, refers to a fusion protein that comprises at least one immunoglobulin region, such as a VL, VH, CL, CH1, CH2, CH3, and CH4 domain. The immunoglobulin region may be a wild type immunoglobulin region or an altered immunoglobulin region. Exemplary immunoglobulin-derived fusion proteins include single chain variable antibody fragment (scFv) (see, e.g., Huston et al., Proc. Natl. Acad. Sci. USA 85: 5879-83, 1988), SMIP™ proteins (see, U.S. Patent Publication Nos. 2003/0133939, 2003/0118592, and 2005/0136049), PIMS proteins (see, PCT Application Publication No. WO 2009/023386), and multi-functional binding proteins (such as SCORPION™ and Xceptor proteins) (see, PCT Application Publication No. WO 2007/146968, U.S. Patent Application Publication No. 2006/0051844, and U.S. Pat. No. 7,166,707).

Additional definitions are provided throughout the present disclosure.

### Polypeptide Heterodimers

In one aspect, the present disclosure provides a polypeptide heterodimer formed by the association of two different single chain polypeptides. The first single chain polypeptide (SCP-I) comprises, consists essentially of, or consists of from one to four binding domains that specifically bind from one to four targets, a hinge (H-I), an immunoglobulin heterodimerization domain (HD-I), and an Fc region portion (FRP-I), whereas the second single chain polypeptide (SCP-II) comprises, consists essentially of, or consists of from zero to four binding domains that specifically bind from zero to four targets, a hinge (H-II), an immunoglobulin heterodimerization domain (HD-II), and an Fc region portion (FRP-II), provided that the polypeptide heterodimer comprises at least two binding domains that specifically bind to one or more targets, for instance, at least two different targets. The H-I and H-II may have the same sequence, but may be different. The HD-I may comprise an immunoglobulin CH1 region, and the HD-II may comprise an immunoglobulin CL region. Alternatively, the HD-I may comprise an immunoglobulin CL region, and the HD-II may comprise an immunoglobulin CH1 region. The FRP-I and FRP-II may have the same sequence, but may be different. The individual components of the polypeptide heterodimers of the present disclosure are described in detail herein.

Binding Domains

As indicated above, the polypeptide heterodimer of the present disclosure comprises two single chain polypeptides: One single chain polypeptide comprises from one to four binding domains that specifically bind from one to four targets, and the other single chain polypeptide of the polypeptide heterodimer comprises from zero to four binding domains that bind from zero to four targets. The total number of binding domains of the polypeptide heterodimer ranges from about two to eight, and the total number of different targets that the binding domains bind ranges from about one to eight, for instance, from two to eight, two to four, two to three or two targets.

If a single chain polypeptide of a polypeptide heterodimer comprises a single binding domain, the binding domain may be located either amino or carboxyl terminal to the Fc region portion of the single chain polypeptide. For example, a single chain polypeptide comprising two binding domains may have one binding domain located amino terminal and the other carboxyl terminal to the Fc region portion of the single chain polypeptide, or both binding domains may be amino terminal or both carboxyl terminal to the Fc region portion. In another example, a single chain polypeptide may comprise three binding domains wherein (a) two binding domains are amino terminal on different single chain proteins and the third binding domain is carboxyl terminal to the Fc region portion on either SCP-I or SCP-II, (b) two binding domains are carboxyl terminal on different single chain proteins and the third binding domain is amino terminal to the Fc region portion on either SCP-I or SCP-II. In still a further example, a polypeptide heterodimer may comprise four binding domains, wherein two binding domains are located amino terminal to the Fc region portion on different chains and the other two binding domains are located carboxyl terminal to the Fc region portion on different chains. Alternatively, in any of these embodiments, two binding domains may be linked to each other in tandem and located on either SCP-I or SCP-II or both, depending on the number of binding domains present—the tandem stacking is used when five to eight binding domains combined are present in SCP-I and SCP-II.

Binding of a target by a binding domain modulates the interaction between the target (e.g., a receptor or a ligand) and another molecule. In certain embodiments, the binding of a target (e.g., a receptor) by a binding domain stimulates certain functions of the target (e.g., signal transduction) or brings different targets closer together for a biological effect (e.g., directing T cells to a tumor which in turn activates the T cells). In certain other embodiments, the binding of a target by a binding domain blocks the interaction between the target and another molecule and thus interferes, reduces or eliminates certain functions of the target.

A target molecule, which is specifically bound by a binding domain contained in a polypeptide heterodimer of the present disclosure, may be found on or in association with a cell of interest (“target cell”). Exemplary target cells include cancer cells, cells associated with an autoimmune disease or disorder or with an inflammatory disease or disorder, B-cells and T-cells. A target molecule may also not be associated with a cell. Exemplary target molecules not associated with a cell include soluble proteins, secreted proteins, deposited proteins, and extracellular structural (matrix) proteins.

In certain embodiments, binding domains of polypeptide heterodimers of the present disclosure specifically bind a target selected from T-cell targets, tumor antigens, B-cell targets, pro-inflammatory cytokines or chemokines, pro-oncogenic cytokines or growth factors, angiogenic agents, Fc receptors, transferrin receptor, receptor tyrosine kinases (RTKs), TNFSFRs, or any combination thereof. For instance, polypeptide heterodimers of the present disclosure specifically bind a T-cell target and a tumor target.

In certain embodiments, a binding domain of a polypeptide heterodimer of the present disclosure specifically binds a T-cell target, such as a TCR complex or a component thereof (e.g., TCRα, TCRβ, CD3γ, CD3δ, and CD3ε), CD28, PD-1, HVEM, BTLA, CD80, CD86, GITR, and TGFBR1.

In certain embodiments, a binding domain of polypeptide heterodimer of the present disclosure specifically binds a TCR complex or a component thereof. For example, in certain embodiments, a binding domain specifically binds to an individual human CD3 chain (e.g., human CD3γ chain, human CD3δ chain, and human CD36ε chain) or a combination of two or more of the individual human CD3 chains (e.g., a complex of human CD3γ and human CD3ε or a complex of human CD3δ and human CD3ε). In certain embodiments, a binding domain specifically binds to a human CD36ε chain. In certain other embodiments, a binding domain specifically binds to one or more of human TCRα, human TCRβ, or a heterodimer formed from human TCRα and human TCRβ. In certain other embodiments, a binding domain of the present disclosure binds to a complex formed from one or more human CD3 chains with one or more human TCR chains, such as a complex formed from a human CD3γ chain, a human CD3δ chain, or a human CD3ε chain with a human TCRα chain or a human TCRβ chain. In any of these embodiments, a polypeptide heterodimer of the present disclosure may also bind a tumor target.

In certain embodiments, one or more binding domains of a polypeptide heterodimer of the present disclosure specifically bind a tumor target, including RON, c-Met, CEACAM-6, PSMA, EpCAM, CEA, PCTA-1, STEAP-1, PSCA, ALCAM (CD166), EphA2, CD151, CA-125, MUC-1, MAGE-1, TROP2, IGF1R, CD44v6, CD151, TGFBR2, GHRHR, GHR, IL-6R, gp130, TNFR2, OSMRβ, Patched-1, Frizzled, Robo1, LTβT, CD80, CD81, CD86, CCR5, HER-3, HER-4, EGFR, CEA, MUC2, MUC3, MUC4, MUC5_{AC}, MUC5_{b}, MUC7, βhCG, Lewis-Y, CD33, CD30, ganglioside GD3, 9-O-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP-2, mesothelin, A33 Antigen, Prostate Stem Cell Antigen (PSCA), Ly-6; desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRvIII, LG, SAS, CD63, B7-H3, or any combination thereof.

In certain embodiments, one or more binding domains of a polypeptide heterodimer of the present disclosure specifically bind a B-cell target, such as CD19, CD20, CD21, CD22, CD30, CD33, CD37, CD38, CD70, CD79b, HLA-DR, or any combination thereof.

In certain embodiments, one or more binding domains of a polypeptide heterodimer of the present disclosure specifically bind a pro-inflammatory cytokine or chemokine, such as TNFα, IL-6, hyperIL-6, IL-2, IL-1, IL-8, IP-10, IFNγ, IFNα, RANKL, FASL, TGFβ, IL7, IL10, IL17A/F, TWEAK, CSF2, IGF1, IGF2 or BLyS/APRIL, or any combination thereof.

In certain embodiments, one or more binding domains of a polypeptide heterodimer of the present disclosure specifically bind a pro-oncogenic cytokine or growth factor, such as HGF, MSP, EGF (including epiregulin, herregulin, β-regulin, neuregulin), HIF-1α, VEGFA, VEGFB, VEGFC, VEGFD, TNFα, IL-6, hyperIL-6, IL-8, Wnt, sHH, TGFβ, PDGF, or any combination thereof.

In certain embodiments, one or more binding domains of a polypeptide heterodimer of the present disclosure specifically bind an angiogenic agent, such as PDGFR, VEGFR1-4, NRP1, Angiopoietin 2, c-Met or any combination thereof.

In certain embodiments, one or more binding domains of a polypeptide heterodimer of the present disclosure specifically bind an Fc receptor, such as CD64, CD32A, CD32B, CD16, FcRn, or any combination thereof.

In certain embodiments, a binding domain of a polypeptide heterodimer of the present disclosure specifically binds a transferrin receptor, such as CD71.

In certain embodiments, one or more binding domains of a polypeptide heterodimer of the present disclosure specifically binds a receptor tyrosine kinase, such as EGFR, EGFRvIII, ErbB2, ErbB3, ErbB4, IGF1R, EphA2, c-Met, RON, or any combination thereof.

In certain embodiments, one or more binding domains of a polypeptide heterodimer of the present disclosure specifically binds a TNFSFR, such as TNFR1, TNFR2, TWEAKR, TACI, BAFF-R, BCMA, FAS, OX40, GITR, 4-1-BB, LTbetaR, HVEM, RANK, or any combination thereof.

In certain embodiments, one or more binding domains of a polypeptide heterodimer of the present disclosure specifically bind hyperIL-6, IL-10, LIGHT, CD40, PDL1, PDL2, or any combination thereof.

In certain embodiments, one or more binding domains of a polypeptide heterodimer of the present disclosure are an agonist of one of the following molecules: IL-10, HLA-G, HGF, IL-35, PD-1, BTLA, TNFR1, TNFR2, DR4, DR5, TWEAKR, FAS, or any combination thereof.

A binding domain may be any peptide that specifically binds a target of interest. Sources of binding domains include antibody variable regions from various species (which can be formatted as antibodies, sFvs, scFvs, Fabs, or soluble VH domain or domain antibodies), including human, rodent, avian, and ovine. Additional sources of binding domains include variable regions of antibodies from other species, such as camelid (from camels, dromedaries, or llamas; Ghahroudi et al. (1997) FEBS Letters 414(3):521-526; Vincke et al. (2009) Journal of Biological Chemistry (2009) 284:3273-3284; Hamers-Casterman et al. (1993) Nature, 363:446 and Nguyen et al. (1998) J. Mol. Biol., 275:413), nurse sharks (Roux et al. (1998) Proc. Nat'l. Acad. Sci. (USA) 95:11804), spotted ratfish (Nguyen et al. (2002) Immunogenetics, 54:39), or lamprey (Herrin et al., (2008) Proc. Nat'l. Acad. Sci. (USA) 105:2040-2045 and Alder et al. (2008) Nature Immunology 9:319-327). These antibodies can apparently form antigen-binding regions using only heavy chain variable region, i.e., these functional antibodies are homodimers of heavy chains only (referred to as “heavy chain antibodies”) (Jespers et al. (2004) Nature Biotechnology 22:1161-1165; Cortez-Retamozo et al. (2004) Cancer Research 64:2853-2857; Baral et al. (2006) Nature Medicine 12:580-584, and Barthelemy et al. (2008) Journal of Biological Chemistry 283:3639-3654).

Exemplary anti-CD3 antibodies from which the binding domain of this disclosure may be derived include Cris-7 monoclonal antibody (Reinherz, E. L. et al. (eds.), Leukocyte typing II., Springer Verlag, New York, (1986)), BC3 monoclonal antibody (Anasetti et al. (1990) J. Exp. Med. 172:1691), OKT3 (Ortho multicenter Transplant Study Group (1985) N. Engl. J. Med. 313:337) and derivatives thereof such as OKT3 ala-ala (Herold et al. (2003) J. Clin. Invest. 11:409), visilizumab (Carpenter et al. (2002) Blood 99:2712), and 145-2C11 monoclonal antibody (Hirsch et al. (1988) J. Immunol. 140: 3766). An exemplary anti-TCR antibody is H57 monoclonal antibody (Lavasani et al. (2007) Scandinavian Journal of Immunology 65:39-47).

An alternative source of binding domains of this disclosure includes sequences that encode random peptide libraries or sequences that encode an engineered diversity of amino acids in loop regions of alternative non-antibody scaffolds, such as fibrinogen domains (see, e.g., Weisel et al. (1985) Science 230:1388), Kunitz domains (see, e.g., U.S. Pat. No. 6,423,498), ankyrin repeat proteins (Binz et al. (2003) Journal of Molecular Biology 332:489-503 and Binz et al. (2004) Nature Biotechnology 22(5):575-582), fibronectin binding domains (Richards et al. (2003) Journal of Molecular Biology 326:1475-1488; Parker et al. (2005) Protein Engineering Design and Selection 18(9):435-444 and Hackel et al. (2008) Journal of Molecular Biology 381:1238-1252), cysteine-knot miniproteins (Vita et al. (1995) Proc. Nat'l. Acad. Sci. (USA) 92:6404-6408; Martin et al. (2002) Nature Biotechnology 21:71-76 and Huang et al. (2005) Structure 13:755-768), tetratricopeptide repeat domains (Main et al. (2003) Structure 11:497-508 and Cortajarena et al. (2008) ACS Chemical Biology 3:161-166), leucine-rich repeat domains (Stumpp et al. (2003) Journal of Molecular Biology 332:471-487), lipocalin domains (see, e.g., WO 2006/095164, Beste et al. (1999) Proc. Nat'l. Acad. Sci. (USA) 96:1898-1903 and Schonfeld et al. (2009) Proc. Nat'l. Acad. Sci. (USA) 106:8198-8203), V-like domains (see, e.g., US Patent Application Publication No. 2007/0065431), C-type lectin domains (Zelensky and Gready (2005) FEBS J. 272:6179; Beavil et al. (1992) Proc. Nat'l. Acad. Sci. (USA) 89:753-757 and Sato et al. (2003) Proc. Nat'l. Acad. Sci. (USA) 100:7779-7784), mAb² or FCAB™ (see, e.g., PCT Patent Application Publication Nos. WO 2007/098934; WO 2006/072620), or the like (Nord et al. (1995) Protein Engineering 8(6):601-608; Nord et al. (1997) Nature Biotechnology 15:772-777; Nord et al. (2001) European Journal of Biochemistry 268(15):4269-4277 and Binz et al. (2005) Nature Biotechnology 23:1257-1268).

Binding domains of this disclosure can be generated as described herein or by a variety of methods known in the art (see, e.g., U.S. Pat. Nos. 6,291,161 and 6,291,158). For example, binding domains of this disclosure may be identified by screening a Fab phage library for Fab fragments that specifically bind to a target of interest (see Hoet et al. (2005) Nature Biotechnol. 23:344). Additionally, traditional strategies for hybridoma development using a target of interest as an immunogen in convenient systems (e.g., mice, HUMAB MOUSE®, TC MOUSE™, KM-MOUSE®, llamas, chicken, rats, hamsters, rabbits, etc.) can be used to develop binding domains of this disclosure.

In certain embodiments, a polypeptide heterodimer comprises a CD86 binding domain, such as a CTLA4 ectodomain, a CD28 ectodomain, or an immunoglobulin variable region binding domain (such as a scFv) specific for CD86 (e.g., from monoclonal antibodies 3D1 or FUN1). In some embodiments, less than an entire ectodomain is used. For example, domains within the CTLA4 ectodomain that bind CD86 and prevent binding of CD86 to CD28 can be used. The CD86 binding domain can block binding of CD86 to CD28 and thereby downregulate T-cell activation.

In certain embodiments, a polypeptide heterodimer comprises an IL-10 agonist, such as IL-10, monoIL-10, or a functional region thereof. “MonoIL-10” refers to an IL-10 molecule having a short linker (GGGSGG, SEQ ID NO:760) that separates the two subdomains of the molecule (amino and carboxyl end domains) so that these subdomains can form an intramolecular dimer.

In certain embodiments, a polypeptide heterodimer comprises an HLA-G agonist, such as an HLA-G5, an HLA-G1, an HLA-G mutein, or a functional region thereof an ectodomain of an HLA-G5, an HLA-G1 or an HLA-G mutein; or an immunoglobulin variable region binding domain (such as an scFv) specific for ILT2, ILT4 or KIR2DL4.

In certain embodiments, a polypeptide heterodimer comprises an HGF agonist, such as an HGF or a sub-domain thereof.

In certain embodiments, a polypeptide heterodimer comprises an IL35 agonist, such as an immunoglobulin variable region binding domain (such as an scFv) specific for IL35R or IL35, or a functional region thereof.

In certain embodiments, a polypeptide heterodimer comprises a LIGHT antagonist, such as an immunoglobulin variable region binding domain (such as an scFv) specific for LIGHT, or a HVEM ectodomain or a functional region thereof.

In certain embodiments, a polypeptide heterodimer comprises a PD-1 agonist, such as an immunoglobulin variable region binding domain (such as an scFv) specific for PD-1, or a PD-1 ligand (e.g. PD-L1 or PD-L2) or a functional region thereof.

In certain embodiments, a polypeptide heterodimer comprises a BTLA agonist, such as an immunoglobulin-like variable region binding domain (such as an scFv) specific for BTLA, or a HVEM ectodomain or a functional region thereof.

In certain embodiments, a polypeptide heterodimer comprises a GITRL antagonist, such as an immunoglobulin-like variable region binding domain (such as an scFv) specific for GITRL, or a GITR ectodomain, soluble GITR, or a functional region thereof.

In certain embodiments, a polypeptide heterodimer comprises a CD40 antagonist, such as an immunoglobulin-like variable region binding domain (such as an scFv) specific for CD40.

In some embodiments, a binding domain is a single chain Fv fragment (scFv) that comprises VH and VL regions specific for a target of interest. In certain embodiments, the V_{H} and V_{L} domains are human. Exemplary VH regions include the VH region of 2E12 (anti-CD28) scFv as set forth in SEQ ID NO:106, the VH region of P2C2 (anti-CD79b) scFv as set forth in SEQ ID NO:184, the VH region of 5D5 (anti-c-Met) scFv as set forth in SEQ ID NO:258, the VH region of A2 (anti-hyperIL-6) scFv as set forth in SEQ ID NO:80, the VH region of 3D1 (anti-CD86) scFv as set forth in SEQ ID NO:92, the VH region of MET021 (anti-c-Met) scFv as set forth in SEQ ID NO:100, the VH region of G19-4 (anti-CD3) scFv as set forth in SEQ ID NO:103, the VH region of HD37 (anti-CD19) scFv as set forth in SEQ ID NO:117, the VH region of M0042 (anti-HLA-DR) scFv as set forth in SEQ ID NO:121, the VH region of BMA031 (anti-TCR) scFv as set forth in SEQ ID NO:828, the VH region of OKT3-M (anti-CD3) scFv as set forth in SEQ ID NO:831, and the VH region of HuM291 (anti-CD3) scFv as set forth in SEQ ID NO:835. The nucleotide sequences encoding the VH regions of the A2 (anti-hyperIL-6) and 3D1 (anti-CD86) scFv's are set forth in SEQ ID NOS:79 and 91, respectively.

Exemplary VL domains are the VL region of 2E12 (anti-CD28) scFv as set forth in SEQ ID NO:107, the VL region of P2C2 (anti-CD79b) scFv as set forth in SEQ ID NO:182, the VL region of 5D5 (anti-c-Met) scFv as set forth in SEQ ID NO:259, the VL region of A2 (anti-hyper IL-6) scFv as set forth in SEQ ID NO:84, the VL region of 3D1 (anti-CD86) scFv as set forth in SEQ ID NO:96, the VL region of MET021 (anti-c-Met) scFv as set forth in SEQ ID NO:101, the VL region of G19-4 (anti-CD3) scFv as set forth in SEQ ID NO:104, the VL region of HD37 (anti-CD19) scFv as set forth in SEQ ID NO:119, the VL region of M0042 (anti-HLA-DR) scFv as set forth in SEQ ID NO:122, the VL region of BMA031 (anti-TCR) scFv as set forth in SEQ ID NO:829, the VL region of OKT3-M (anti-CD3) scFv as set forth in SEQ ID NO:833, and the VL region of HuM291 (anti-CD3) scFv as set forth in SEQ ID NO:836. The nucleotide sequences encoding the VL regions of the A2 (anti-hyperIL-6) and 3D1 (anti-CD86) scFv's are set forth in SEQ ID NOS:83 and 95, respectively.

In some embodiments, a binding domain is a single chain Fv fragment (scFv) that comprises V_{H} and V_{L} domains specific for a TCR complex or a component thereof. In certain embodiments, the V_{H} and V_{L} domains are human or humanized V_{H} and V_{L} domains. Exemplary V_{H} domains include BC3 (anti-CD3) V_{H}, OKT3 (anti-CD3) V_{H}, H57 (anti-TCR) V_{H}, and 2C11 (anti-CD3) V_{H} domains as set forth in SEQ ID NOS:301, 303, 313, and 317, respectively. Further exemplary V_{H} domains include Cris-7 (anti-CD3) V_{H} domains, such as those set forth in SEQ ID NOS:327 and 331-333. Exemplary V_{L} domains are BC3 (anti-CD3) V_{L}, OKT3 (anti-CD3) V_{L}, H57 (anti-TCR) V_{L}, and 2C11 (anti-CD3) V_{L} domains as set forth in SEQ ID NOS:302, 304, 315 and 318, respectively. Further exemplary V_{L} domains include Cris-7 (anti-CD3) V_{L} domains, such as those set forth in SEQ ID NOS:328, 329 and 330.

In certain embodiments, a binding domain comprises or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (V_{L}) or to a heavy chain variable region (V_{H}), or both, wherein each CDR comprises zero changes or at most one, two, or three changes, from a monoclonal antibody or fragment or derivative thereof that specifically binds to a target of interest (e.g., c-Met, RON, CD28, CD79b, CD3ε, TCRα, TCRβ, hyper IL-6, CD86, CD19, and HLA-DR) and such mutant or derivative still binds its target.

In certain embodiments, a binding domain VH or VL region of the present disclosure can be derived from or based on a VH or VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH or VL of a known monoclonal antibody. The insertion(s), deletion(s) or substitution(s) may be anywhere in the VH region, including at the amino- or carboxy-terminus or both ends of this region, provided that each CDR comprises zero changes or at most one, two, or three changes and provided that a binding domain containing the modified VH or VL region can still specifically bind its target with an affinity about the same as the wild type binding domain.

The VH and VL domains may be arranged in either orientation (i.e., from amino-terminus to carboxyl terminus, VH-VL or VL-VH) and may be joined by an amino acid sequence (e.g., having a length of about five to about 35 amino acids) capable of providing a spacer function such that the two sub-binding domains can interact to form a functional binding domain. In certain embodiments, an amino acid sequence that joins the VH and VL domains (also referred to herein as a “linker”) includes those belonging to the (GlyₙSer) family, such as (Gly₃Ser)ₙ(Gly₄Ser)₁, (Gly₃Ser)₁(Gly₄Ser)ₙ, (Gly₃Ser)ₙ(Gly₄Ser)ₙ, or (Gly₄Ser)ₙ, wherein n is an integer of 1 to 5. In certain embodiments, the linker is GGGGSGGGGS GGGGS (SEQ ID NO:183) or GGGGSGGGGSGGGGSGGGGS (SEQ ID NO:108). An additional exemplary linker is GGGGSGGGGSGGGGAS (SEQ ID NO:739). In certain embodiments, these (GlyₙSer)-based linkers are used to link the VH and VL domains in a binding domain, but are not used to link a binding domain to an immunoglobulin heterodimerization domain or to an Fc region portion.

Exemplary binding domains specific for CD28 include a 2E12 scFv as set forth in SEQ ID NO:109, binding domains specific for CD79b include a P2C2 scFv as set forth in SEQ ID NO:185; binding domains specific for c-Met include a 5D5 scFv as set forth in SEQ ID NO:257; binding domains specific for RON include a 4C04 scFv as set forth in SEQ ID NO:261 and a 11H09 scFv as set forth in SEQ ID NO:265; binding domains specific for hyperIL-6 include an A2 scFv as set forth in SEQ ID NO:86; binding domains specific for CD86 include a 3D1 scFv as set forth in SEQ ID NO:98; binding domains specific for HLA-DR include an M0042 scFv as set forth in SEQ ID NO:120; binding domains specific for CD3 include a G19-4 scFv as set forth in SEQ ID NO:102, an OKT3-M scFv as set forth in SEQ ID NO:834, and a HuM291 scFv as set forth in SEQ ID NO:837; binding domain specific for CD19 include a H37 scFv as set forth in SEQ ID NO:105; and binding domains specific for c-Met include an MET021 scFv as set forth in SEQ ID NO:120 (with the light chain CDR1, CDR2, CDR3 and heavy chain CDR1, CDR2 and CDR3 as set forth in SEQ ID NOS:296-298 and 464-466, respectively). The nucleotide sequences encoding the A2 (anti-hyperIL6) and 3D1 (anti-CD86) scFv's are set forth in SEQ ID NOS:85 and 97, respectively. Exemplary binding domains that bind a TCR complex or a component thereof include a BMA031 scFv as set forth in SEQ ID NO:830 and other scFv's as set forth in SEQ ID NOS:310, 311, 312, 319, and 334-340.

Additional exemplary binding domains include a PDL2 ectodomain as set forth in SEQ ID NO:88 and monoIL-10 as set forth in SEQ ID NO:90. The nucleotide sequences encoding the PDL2 ectodomain and monoIL-10 are set forth in SEQ ID NOS:87 and 89, respectively.

The light chain amino acid sequence of the 4C04 (anti-RON) scFv is set forth in SEQ ID NO:602, and its CDR1, CDR2, and CDR3 are set forth in SEQ ID NOS:604-606, respectively. The heavy chain amino acid sequence of the 4C04 (anti-RON) scFv is set forth in SEQ ID NO:603, and its CDR1, CDR2, and CDR3 are set forth in SEQ ID NOS:607-609, respectively.

The light chain amino acid sequence of the 11H09 (anti-RON) scFv is set forth in SEQ ID NO:610, and its CDR1, CDR2, and CDR3 are set forth in SEQ ID NOS:612-614, respectively. The heavy chain amino acid sequence of the 11H09 (anti-RON) scFv is set forth in SEQ ID NO:611, and its CDR1, CDR2, and CDR3 are set forth in SEQ ID NOS:615-617, respectively.

A binding domain may be located either at amino terminal or carboxyl terminal to the Fc region portion of a single chain polypeptide of the present disclosure. In certain embodiments, the binding domain is located at the amino terminus of a single chain polypeptide. In certain other embodiments, the binding domain is located at the carboxyl terminus of a single chain polypeptide. In certain other embodiments, a binding domain is located at both the amino and carboxyl terminus of a single chain polypeptide.

Heterodimerization Domains

As indicated above, a polypeptide heterodimer of the present disclosure comprises an immunoglobulin heterodimerization domain in each polypeptide chain. The immunoglobulin heterodimerization domains in the two single chain polypeptides of a polypeptide heterodimer are different from each other and thus may be differentially modified to facilitate heterodimerization of both chains and to minimize homodimerization of either chain. As shown in the examples, immunoglobulin heterodimerization domains provided herein allow for efficient heterodimerization between different polypeptides and facilitate purification of the resulting polypeptide heterodimers.

As provided herein, immunoglobulin heterodimerization domains useful for promoting heterodimerization of two different single chain polypeptides (e.g., one short and one long) according to the present disclosure include immunoglobulin CH1 and CL domains, for instance, human CH1 and CL domains. In certain embodiments, an immunoglobulin heterodimerization domain is a wild type CH1 region, such as a wild type IgG1, IgG2, IgG3, IgG4, IgA1, IgA2, IgD, IgE, or IgM CH1 region. In further embodiments, an immunoglobulin heterodimerization domain is a wild type human IgG1, IgG2, IgG3, IgG4, IgA1, IgA2, IgD, IgE, or IgM CH1 region as set forth in SEQ ID NOS:114, 186-192 and 194, respectively. In certain embodiments, an immunoglobulin heterodimerization domain is a wild type human IgG1 CH1 region as set forth in SEQ ID NO:114.

In further embodiments, an immunoglobulin heterodimerization domain is an altered immunoglobulin CH1 region, such as an altered IgG1, IgG2, IgG3, IgG4, IgA1, IgA2 IgD, IgE, or IgM CH1 region. In certain embodiments, an immunoglobulin heterodimerization domain is an altered human IgG1, IgG2, IgG3, IgG4, IgA1, IgA2, IgD, IgE, or IgM CH1 region. In still further embodiments, a cysteine residue of a wild type CH1 region (e.g., a human CH1) involved in forming a disulfide bond with a wild type immunoglobulin CL domain (e.g., a human CL) is deleted or substituted in the altered immunoglobulin CH1 region such that a disulfide bond is not formed between the altered CH1 region and the wild type CL domain.

In certain embodiments, an immunoglobulin heterodimerization domain is a wild type CL domain, such as a wild type Cκ domain or a wild type Cλ domain. In particular embodiments, an immunoglobulin heterodimerization domain is a wild type human Cκ or human Cλ domain as set forth in SEQ ID NOS:112 and 113, respectively. In further embodiments, an immunoglobulin heterodimerization domain is an altered immunoglobulin CL domain, such as an altered Cκ or Cλ domain, for instance, an altered human Cκ or human Cλ domain.

In certain embodiments, a cysteine residue of a wild type CL domain (e.g., a human CL) involved in forming a disulfide bond with a wild type immunoglobulin CH1 region (e.g., a human CH1) is deleted or substituted in the altered immunoglobulin CL domain. Such altered CL domains may further comprise an amino acid deletion at their amino termini. An exemplary Cκ domain is set forth in SEQ ID NO:141, in which the first arginine and the last cysteine of the wild type human Ck domain are both deleted. In certain embodiments, only the last cysteine of the wild type human Ck domain is deleted in the altered Ck domain because the first arginine deleted from the wild type human Ck domain may be provided by a linker that has an arginine at its carboxyl terminus and links the amino terminus of the altered Ck domain with another domain (e.g., an Fc region portion). An exemplary Cλ domain is set forth in SEQ ID NO:140, in which the first arginine of a wild type human Cλ domain is deleted and the cysteine involved in forming a disulfide bond with a cysteine in a CH1 region is substituted by a serine.

In further embodiments, an immunoglobulin heterodimerization domain is an altered Cκ domain that contains one or more amino acid substitutions, as compared to a wild type Cκ domain, at positions that may be involved in forming the interchain-hydrogen bond network at a Cκ-Cκ interface. For example, in certain embodiments, an immunoglobulin heterodimerization domain is an altered human Cκ domain having one or more amino acids at positions N29, N30, Q52, V55, T56, S68 or T70 that are substituted with a different amino acid. The numbering of the amino acids is based on their positions in the altered human Cκ sequence as set forth in SEQ ID NO:141. In certain embodiments, an immunoglobulin heterodimerization domain is an altered human Cκ domain having one, two, three or four amino acid substitutions at positions N29, N30, V55, or T70. The amino acid used as a substitute at the above-noted positions may be an alanine, or an amino acid residue with a bulk side chain moiety such as arginine, tryptophan, tyrosine, glutamate, glutamine, or lysine. Additional amino acid residues that may be used to substitute amino acid residues of the wild type human Ck sequence at the above noted positions (e.g., N30) include aspartate, methionine, serine and phenyalanine. Exemplary altered human Cκ domains are set forth in SEQ ID NOS:142-178. Altered human Cκ domains are those that facilitate heterodimerization with a CH1 region, but minimize homodimerization with another Cκ domain. Representative altered human Cκ domains are set forth in SEQ ID NOS:160 (N29W V55A T70A), 161 (N29Y V55A T70A), 202 (T70E N29A N30A V55A), 167 (N30R V55A T70A), 168 (N30K V55A T70A), 170 (N30E V55A T70A), 172 (V55R N29A N30A), 175 (N29W N30Y V55A T70E), 176 (N29Y N30Y V55A T70E), 177 (N30E V55A T70E), 178 (N30Y V55A T70E), 838 (N30D V55A T70E), 839 (N30M V55A T70E), 840 (N30S V55A T70E), and 841 (N30F V55A T70E).

In certain embodiments, in addition to or alternative to the mutations in Ck domains described herein, both the immunoglobulin heterodimerization domains (i.e., immunoglobulin CH1 and CL domains) of a polypeptide heterodimer have mutations so that the resulting immunoglobulin heterodimerization domains form salt bridges (i.e., ionic interactions) between the amino acid residues at the mutated sites. For example, the immunoglobulin heterodimerization domains of a polypeptide heterodimer may be a mutated CH1 domain in combination with a mutated Ck domain. In the mutated CH1 domain, valine at position 68 (V68) of the wild type human CH1 domain is substituted by an amino acid residue having a negative charge (e.g., aspartate or glutamate), whereas leucine at position 29 (L29) of a mutated human Ck domain in which the first arginine and the last cysteine have been deleted is substituted by an amino acid residue having a positive charge (e.g., lysine, arginine or histidine). The charge-charge interaction between the amino acid residue having a negative charge of the resulting mutated CH1 domain and the amino acid residue having a positive charge of the resulting mutated Ck domain forms a salt bridge, which stabilizes the heterodimeric interface between the mutated CH1 and Ck domains. Alternatively, V68 of the wild type CH1 may be substituted by an amino acid residue having a positive charge, whereas L29 of a mutated human Ck domain in which the first arginine and the last cysteine have been deleted may be substituted by an amino acid residue having a negative charge. Exemplary mutated CH1 sequences in which V68 is substituted by an amino acid with either a negative or positive charge are set forth in SEQ ID NOS:844 and 845. Exemplary mutated Ck sequences in which L29 is substituted by an amino acid with either a negative or positive charge are set forth in SEQ ID NOS:842 and 843.

Positions other than V68 of human CH1 domain and L29 of human Ck domain may be substituted with amino acids having opposite charges to produce ionic interactions between the amino acids in addition or alternative to the mutations in V68 of CH1 domain and L29 of Ck domain. Such positions can be identified by any suitable method, including random mutagenesis, analysis of the crystal structure of the CH1-Ck pair to identify amino acid residues at the CH1-Ck interface, and further identifying suitable positions among the amino acid residues at the CH1-Ck interface using a set of criteria (e.g., propensity to engage in ionic interactions, proximity to a potential partner residue, etc.).

In certain embodiments, polypeptide heterodimers of the present disclosure contain only one pair of immunoglobulin heterodimerization domains. For example, a first chain of a polypeptide heterodimer may comprise a CH1 region as an immunoglobulin heterodimerization domain, while a second chain may comprise a CL domain (e.g., a Cκ or Cλ) as an immunoglobulin heterodimerization domain. Alternatively, a first chain may comprise a CL region (e.g., a Cκ or Cλ) as an immunoglobulin heterodimerization domain, while a second chain may comprise a CH1 region as an immunoglobulin heterodimerization domain. As set forth herein, the immunoglobulin heterodimerization domains of the first and second chains are capable of associating to form a polypeptide heterodimer of this disclosure.

In certain other embodiments, polypeptide heterodimers of the present disclosure may have two pairs of immunoglobulin heterodimerization domains. For example, a first chain of a polypeptide heterodimer may comprise two CH1 regions, while a second chain may have two CL domains that associate with the two CH1 regions in the first chain. Alternatively, a first chain may comprise two CL domains, while a second chain may have two CH1 regions that associate with the two CL domains in the first chain. In certain embodiments, a first chain polypeptide comprises a CH1 region and a CL domain, while a second chain polypeptide comprises a CL domain and a CH1 region that associate with the CH1 region and the CL domain, respectively, of the first chain polypeptide.

In the embodiments where a polypeptide heterodimer comprises only one heterodimerization pair (i.e., one immunoglobulin heterodimerization domain in each chain), the immunoglobulin heterodimerization domain of each chain may be located amino terminal to the Fc region portion of that chain. Alternatively, the immunoglobulin heterodimerization domain in each chain may be located carboxyl terminal to the Fc region portion of that chain.

In the embodiments where a polypeptide heterodimer comprises two heterodimerization pairs (i.e., two immunoglobulin heterodimerization domains in each chain), both immunoglobulin heterodimerization domains in each chain may be located amino terminal to the Fc region portion of that chain. Alternatively, both immunoglobulin heterodimerization domains in each chain may be located carboxyl terminal to the Fc region portion of that chain. In further embodiments, one immunoglobulin heterodimerization domain in each chain may be located amino terminal to the Fc region portion of that chain, while the other immunoglobulin heterodimerization domain of each chain may be located carboxyl terminal to the Fc region portion of that chain. In other words, in those embodiments, the Fc region portion is interposed between the two immunoglobulin heterodimerization domains of each chain.

Fc Region Portion

As indicated herein, polypeptide heterodimers of the present disclosure comprise an Fc region constant domain portion (also referred to as an Fc region portion) in each polypeptide chain. The inclusion of an Fc region portion slows clearance of the heterodimers from circulation after administration to a subject. By mutations or other alterations, the Fc region portion further enables relatively easy modulation of heterodimer polypeptide effector functions (e.g., ADCC, ADCP, CDC, complement fixation and binding to Fc receptors), which can either be increased or decreased depending on the disease being treated, as known in the art and described herein. In certain embodiments, an Fc region portion of polypeptide heterodimers of the present disclosure will be capable of mediating one or more of these effector functions.

An Fc region portion present in single chain polypeptides that form part of the polypeptide heterodimers of the present disclosure may comprise a CH2 domain, a CH3 domain, a CH4 domain or any combination thereof. For example, an Fc region portion may comprise a CH2 domain, a CH3 domain, both CH2 and CH3 domains, both CH3 and CH4 domains, two CH3 domains, a CH4 domain, or two CH4 domains.

A CH2 domain that may form an Fc region portion of a single chain polypeptide of a heterodimer of the present disclosure may be a wild type immunoglobulin CH2 domain or an altered immunoglobulin CH2 domain thereof from certain immunoglobulin classes or subclasses (e.g., IgG1, IgG2, IgG3, IgG4, IgA1, IgA2, or IgD) and from various species (including human, mouse, rat, and other mammals).

In certain embodiments, a CH2 domain is a wild type human immunoglobulin CH2 domain, such as wild type CH2 domains of human IgG1, IgG2, IgG3, IgG4, IgA1, IgA2, or IgD, as set forth in SEQ ID NOS:115, 199-201 and 195-197, respectively. In certain embodiments, the CH2 domain is a wild type human IgG1 CH2 domain as set forth in SEQ ID NO:115.

In certain embodiments, a CH2 domain is an altered immunoglobulin CH2 region (e.g., an altered human IgG1 CH2 domain) that comprises an amino acid substitution at the asparagine of position 297 (e.g., asparagine to alanine). Such an amino acid substitution reduces or eliminates glycosylation at this site and abrogates efficient Fc binding to FcγR and C1q. The sequence of an altered human IgG1 CH2 domain with an Asn to Ala substitution at position 297 is set forth in SEQ ID NO:324.

In certain embodiments, a CH2 domain is an altered immunoglobulin CH2 region (e.g., an altered human IgG1 CH2 domain) that comprises at least one substitution or deletion at positions 234 to 238. For example, an immunoglobulin CH2 region can comprise a substitution at position 234, 235, 236, 237 or 238, positions 234 and 235, positions 234 and 236, positions 234 and 237, positions 234 and 238, positions 234-236, positions 234, 235 and 237, positions 234, 236 and 238, positions 234, 235, 237, and 238, positions 236-238, or any other combination of two, three, four, or five amino acids at positions 234-238. In addition or alternatively, an altered CH2 region may comprise one or more (e.g., two, three, four or five) amino acid deletions at positions 234-238, for instance, at one of position 236 or position 237 while the other position is substituted. The above-noted mutation(s) decrease or eliminate the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 has been replaced with one or more alanine residues. In further embodiments, only one of the amino acid residues at positions 234-238 have been deleted while one or more of the remaining amino acids at positions 234-238 can be substituted with another amino acid (e.g., alanine or serine).

In certain other embodiments, a CH2 domain is an altered immunoglobulin CH2 region (e.g., an altered human IgG1 CH2 domain) that comprises one or more amino acid substitutions at positions 253, 310, 318, 320, 322, and 331. For example, an immunoglobulin CH2 region can comprise a substitution at position 253, 310, 318, 320, 322, or 331, positions 318 and 320, positions 318 and 322, positions 318, 320 and 322, or any other combination of two, three, four, five or six amino acids at positions 253, 310, 318, 320, 322, and 331. The above-noted mutation(s) decrease or eliminate the complement-dependent cytotoxicity (CDC) of a polypeptide heterodimer that comprises the altered CH2 domain.

In certain other embodiments, in addition to the amino acid substitution at position 297, an altered CH2 region (e.g., an altered human IgG1 CH2 domain) can further comprise one or more (e.g., two, three, four, or five) additional substitutions at positions 234-238. For example, an immunoglobulin CH2 region can comprise a substitution at positions 234 and 297, positions 234, 235, and 297, positions 234, 236 and 297, positions 234-236 and 297, positions 234, 235, 237 and 297, positions 234, 236, 238 and 297, positions 234, 235, 237, 238 and 297, positions 236-238 and 297, or any combination of two, three, four, or five amino acids at positions 234-238 in addition to position 297. In addition or alternatively, an altered CH2 region may comprise one or more (e.g., two, three, four or five) amino acid deletions at positions 234-238, such as at position 236 or position 237. The additional mutation(s) decreases or eliminates the antibody-dependent cell-mediated cytotoxicity (ADCC) activity or Fc receptor-binding capability of a polypeptide heterodimer that comprises the altered CH2 domain. In certain embodiments, the amino acid residues at one or more of positions 234-238 have been replaced with one or more alanine residues. In further embodiments, only one of the amino acid residues at positions 234-238 has been deleted while one or more of the remaining amino acids at positions 234-238 can be substituted with another amino acid (e.g., alanine or serine).

In certain embodiments, in addition to one or more (e.g., 2, 3, 4, or 5) amino acid substitutions at positions 234-238, a mutated CH2 region (e.g., an altered human IgG1 CH2 domain) in a fusion protein of the present disclosure may contain one or more (e.g., 2, 3, 4, 5, or 6) additional amino acid substitutions (e.g., substituted with alanine) at one or more positions involved in complement fixation (e.g., at positions 1253, H310, E318, K320, K322, or P331). Examples of mutated immunoglobulin CH2 regions include human IgG1, IgG2, IgG4 and mouse IgG2a CH2 regions with alanine substitutions at positions 234, 235, 237 (if present), 318, 320 and 322. An exemplary mutated immunoglobulin CH2 region is mouse IGHG2c CH2 region with alanine substitutions at L234, L235, G237, E318, K320, and K322 (SEQ ID NO:314).

In still further embodiments, in addition to the amino acid substitution at position 297 and the additional deletion(s) or substitution(s) at positions 234-238, an altered CH2 region (e.g., an altered human IgG1 CH2 domain) can further comprise one or more (e.g., two, three, four, five, or six) additional substitutions at positions 253, 310, 318, 320, 322, and 331. For example, an immunoglobulin CH2 region can comprise a (1) substitution at position 297, (2) one or more substitutions or deletions or a combination thereof at positions 234-238, and one or more (e.g., 2, 3, 4, 5, or 6) amino acid substitutions at positions 1253, H310, E318, K320, K322, and P331, such as one, two, three substitutions at positions E318, K320 and K322. The amino acids at the above-noted positions may be substituted by alanine or serine.

In certain embodiments, an immunoglobulin CH2 region polypeptide comprises: (i) an amino acid substitution at the asparagines of position 297 and one amino acid substitution at position 234, 235, 236 or 237; (ii) an amino acid substitution at the asparagine of position 297 and amino acid substitutions at two of positions 234-237; (iii) an amino acid substitution at the asparagine of position 297 and amino acid substitutions at three of positions 234-237; (iv) an amino acid substitution at the asparagine of position 297, amino acid substitutions at positions 234, 235 and 237, and an amino acid deletion at position 236; (v) amino acid substitutions at three of positions 234-237 and amino acid substitutions at positions 318, 320 and 322; or (vi) amino acid substitutions at three of positions 234-237, an amino acid deletion at position 236, and amino acid substitutions at positions 318, 320 and 322.

Exemplary altered immunoglobulin CH2 regions with amino acid substitutions at the asparagine of position 297 include: human IgG1 CH2 region with alanine substitutions at L234, L235, G237 and N297 and a deletion at G236 (SEQ ID NO:325), human IgG2 CH2 region with alanine substitutions at V234, G236, and N297 (SEQ ID NO:326), human IgG4 CH2 region with alanine substitutions at F234, L235, G237 and N297 and a deletion of G236 (SEQ ID NO:322), human IgG4 CH2 region with alanine substitutions at F234 and N297 (SEQ ID NO:343), human IgG4 CH2 region with alanine substitutions at L235 and N297 (SEQ ID NO:344), human IgG4 CH2 region with alanine substitutions at G236 and N297 (SEQ ID NO:345), and human IgG4 CH2 region with alanine substitutions at G237 and N297 (SEQ ID NO:346).

In certain embodiments, in addition to the amino acid substitutions described above, an altered CH2 region (e.g., an altered human IgG1 CH2 domain) may contain one or more additional amino acid substitutions at one or more positions other than the above-noted positions. Such amino acid substitutions may be conservative or non-conservative amino acid substitutions. For example, in certain embodiments, P233 may be changed to E233 in an altered IgG2 CH2 region (see, e.g., SEQ ID NO:326). In addition or alternatively, in certain embodiments, the altered CH2 region may contain one or more amino acid insertions, deletions, or both. The insertion(s), deletion(s) or substitution(s) may anywhere in an immunoglobulin CH2 region, such as at the N- or C-terminus of a wild type immunoglobulin CH2 region resulting from linking the CH2 region with another region (e.g., a binding domain or an immunoglobulin heterodimerization domain) via a hinge.

In certain embodiments, an altered CH2 region in a polypeptide heterodimer of the present disclosure comprises or is a sequence that is at least 90%, at least 91%, at least 92%, at least 93%, at least 94%, at least 95%, at least 96%, at least 97%, at least 98%, at least 99% identical to a wild type immunoglobulin CH2 region, such as the CH2 region of wild type human IgG1, IgG2, or IgG4, or mouse IgG2a (e.g., IGHG2c).

An altered immunoglobulin CH2 region in a polypeptide heterodimer of the present disclosure may be derived from a CH2 region of various immunoglobulin isotypes, such as IgG1, IgG2, IgG3, IgG4, IgA1, IgA2, and IgD, from various species (including human, mouse, rat, and other mammals). In certain embodiments, an altered immunoglobulin CH2 region in a fusion protein of the present disclosure may be derived from a CH2 region of human IgG1, IgG2 or IgG4, or mouse IgG2a (e.g., IGHG2c), whose sequences are set forth in SEQ ID NOS:115, 199, 201 and 320.

In certain embodiments, an altered CH2 domain is a human IgG1 CH2 domain with alanine substitutions at positions 235, 318, 320, and 322 (i.e., a human IgG1 CH2 domain with L235A, E318A, K320A and K322A substitutions) (SEQ ID NO:595), and optionally an N297 mutation (e.g., to alanine). In certain other embodiments, an altered CH2 domain is a human IgG1 CH2 domain with alanine substitutions at positions 234, 235, 237, 318, 320 and 322 (i.e., a human IgG1 CH2 domain with L234A, L235A, G237A, E318A, K320A and K322A substitutions) (SEQ ID NO:596), and optionally an N297 mutation (e.g., to alanine)

In certain embodiments, an altered CH2 domain is an altered human IgG1 CH2 domain with mutations known in the art that enhance immunological activities such as ADCC, ADCP, CDC, complement fixation, Fc receptor binding, or any combination thereof.

The CH3 domain that may form an Fc region portion of a single chain polypeptide of a heterodimer of the present disclosure may be a wild type immunoglobulin CH3 domain or an altered immunoglobulin CH3 domain thereof from certain immunoglobulin classes or subclasses (e.g., IgG1, IgG2, IgG3, IgG4, IgA1, IgA2, IgD, IgE, IgM) of various species (including human, mouse, rat, and other mammals). In certain embodiments, a CH3 domain is a wild type human immunoglobulin CH3 domain, such as wild type CH3 domains of human IgG1, IgG2, IgG3, IgG4, IgA1, IgA2, IgD, IgE, or IgM as set forth in SEQ ID NOS:116, 208-210, 204-207, and 212, respectively. In certain embodiments, the CH3 domain is a wild type human IgG1 CH3 domain as set forth in SEQ ID NO:116. In certain embodiments, a CH3 domain is an altered human immunoglobulin CH3 domain, such as an altered CH3 domain based on or derived from a wild-type CH3 domain of human IgG1, IgG2, IgG3, IgG4, IgA1, IgA2, IgD, IgE, or IgM antibodies. For example, an altered CH3 domain may be a human IgG1 CH3 domain with one or two mutations at positions H433 and N434 (positions are numbered according to EU numbering). The mutations in such positions may be involved in complement fixation. In certain other embodiments, an altered CH3 domain may be a human IgG1 CH3 domain but with one or two amino acid substitutions at position F405 or Y407. The amino acids at such positions are involved in interacting with another CH3 domain. In certain embodiments, an altered CH3 domain may be an altered human IgG1 CH3 domain with its last lysine deleted. The sequence of this altered CH3 domain is set forth in SEQ ID NO:761.

In certain embodiments, a polypeptide heterodimer comprises a CH3 pair that comprises so called “knobs-into-holes” mutations (see, Marvin and Zhu, Acta Pharmacologica Sinica 26:649-58, 2005; Ridgway et al., Protein Engineering 9:617-21, 1966). More specifically, mutations may be introduced into each of the two CH3 domains so that the steric complementarity required for CH3/CH3 association obligates these two CH3 domains to pair with each other. For example, a CH3 domain in one single chain polypeptide of a polypeptide heterodimer may contain a T366W mutation (a “knob” mutation, which substitutes a small amino acid with a larger one), and a CH3 domain in the other single chain polypeptide of the polypeptide heterodimer may contain a Y407A mutation (a “hole” mutation, which substitutes a large amino acid with a smaller one). Other exemplary knobs-into-holes mutations include (1) a T366Y mutation in one CH3 domain and a Y407T in the other CH3 domain, and (2) a T366W mutation in one CH3 domain and T366S, L368A and Y407V mutations in the other CH3 domain.

The CH4 domain that may form an Fc region portion of a single chain polypeptide of a heterodimer of the present disclosure may be a wild type immunoglobulin CH4 domain or an altered immunoglobulin CH4 domain thereof from IgE or IgM molecules. In certain embodiments, the CH4 domain is a wild type human immunoglobulin CH4 domain, such as wild type CH4 domains of human IgE and IgM molecules as set forth in SEQ ID NOS:213 and 214, respectively. In certain embodiments, a CH4 domain is an altered human immunoglobulin CH4 domain, such as an altered CH4 domain based on or derived from a CH4 domain of human IgE or IgM molecules, which have mutations that increase or decrease an immunological activity known to be associated with an IgE or IgM Fc region.

In certain embodiments, an Fc region constant domain portion in heterodimers of the present disclosure comprises a combination of CH2, CH3 or CH4 domains (i.e., more than one constant sub-domain selected from CH2, CH3 and CH4). For example, the Fc region portion may comprise CH2 and CH3 domains or CH3 and CH4 domains. In certain other embodiments, the Fc region portion may comprise two CH3 domains and no CH2 or CH4 domains (i.e., only two or more CH3). The multiple constant sub-domains that form an Fc region portion may be based on or derived from the same immunoglobulin molecule, or the same class or subclass immunoglobulin molecules. In certain embodiments, the Fc region portion is an IgG CH2CH3 (e.g., IgG1 CH2CH3, IgG2 CH2CH3, and IgG4 CH2CH3) and may be a human (e.g., human IgG1, IgG2, and IgG4) CH2CH3. For example, in certain embodiments, the Fc region portion comprises (1) wild type human IgG1 CH2 and CH3 domains, (2) human IgG1 CH2 with N297A substitution (i.e., CH2(N297A)) and wild type human IgG1 CH3, or (3) human IgG1 CH2(N297A) and an altered human IgG1 CH3 with the last lysine deleted.

Alternatively, the multiple constant sub-domains may be based on or derived from different immunoglobulin molecules, or different classes or subclasses immunoglobulin molecules. For example, in certain embodiments, an Fc region portion comprises both human IgM CH3 domain and human IgG1 CH3 domain. The multiple constant sub-domains that form an Fc region portion may be directly linked together or may be linked to each other via one or more (e.g., about 2-10) amino acids.

Exemplary Fc region portions are set forth in SEQ ID NOS:305-309, 321, 323, 341, 342, and 762.

In certain embodiments, the Fc region portions of both single chain polypeptides of a polypeptide heterodimer are identical to each other. In certain other embodiments, the Fc region portion of one single chain polypeptide of a polypeptide heterodimer is different from the Fc region portion of the other single chain polypeptide of the heterodimer. For example, one Fc region portion may contain a CH3 domain with a “knob” mutation, whereas the other Fc region portion may contain a CH3 domain with a “hole” mutation.

Hinge

A hinge region contained in a single chain polypeptide of a polypeptide heterodimer according to the present disclosure may be located (a) immediately amino terminal to an Fc region portion (e.g., depending on the isotype, amino terminal to a CH2 domain wherein the Fc region portion is a CH2CH3, or amino terminal to a CH3 domain wherein the Fc region portion is a CH3CH4), (b) interposed between and connecting a binding domain (e.g., scFv) and an immunoglobulin heterodimerization domain, (c) interposed between and connecting an immunoglobulin heterodimerization domain and an Fc region portion (e.g., wherein the Fc region portion is a CH2CH3 or a CH3CH4, depending on the isotype or isotypes), (d) interposed between and connecting an Fc region portion and a binding domain, (e) at the amino terminus of the single chain polypeptide, or (f) at the carboxyl terminus of the single chain polypeptide. The single chain polypeptide comprising a hinge region as described herein will be capable of associating with a different single chain fusion polypeptide to form a polypeptide heterodimer provided herein, and the polypeptide heterodimer formed will contain a binding domain that retains its target specificity or its specific target binding affinity.

In certain embodiments, a hinge present in a single chain polypeptide that forms a polypeptide heterodimer with another single chain polypeptide may be an immunoglobulin hinge region, such as a wild type immunoglobulin hinge region or an altered immunoglobulin hinge region thereof.

In certain embodiments, a hinge is a wild type human immunoglobulin hinge region (e.g., human immunoglobulin hinge regions as set forth in SEQ ID NOS:215-221). In certain other embodiments, one or more amino acid residues may be added at the amino- or carboxy-terminus of a wild type immunoglobulin hinge region as part of a fusion protein construct design. For example, additional junction amino acid residues at the hinge amino-terminus can be “RT,” “RSS,” “TG,” or “T”, or at the hinge carboxy-terminus can be “SG”, or a hinge deletion can be combined with an addition, such as ΔP with “SG” added at the carboxyl terminus.

In certain embodiments, a hinge is an altered immunoglobulin hinge in which one or more cysteine residues in a wild type immunoglobulin hinge region is substituted with one or more other amino acid residues (e.g., serine or alanine). For example, a hinge may be an altered immunoglobulin hinge based on or derived from a wild type human IgG1 hinge as set forth in SEQ ID NO:667, which from amino terminus to carboxyl terminus comprises the upper hinge region (EPKSCDKTHT, SEQ ID NO:227) and the core hinge region (CPPCP, SEQ ID NO:228). Exemplary altered immunoglobulin hinges include an immunoglobulin human IgG1 hinge region having one, two or three cysteine residues found in a wild type human IgG1 hinge substituted by one, two or three different amino acid residues (e.g., serine or alanine). An altered immunoglobulin hinge may additionally have a proline substituted with another amino acid (e.g., serine or alanine). For example, the above-described altered human IgG1 hinge may additionally have a proline located carboxyl terminal to the three cysteines of wild type human IgG1 hinge region substituted by another amino acid residue (e.g., serine, alanine). In one embodiment, the prolines of the core hinge region are not substituted. Exemplary altered immunoglobulin hinges are set forth in SEQ ID NOS: 229-240, 255, 664-677, and 748-759. An example of an altered IgG1 hinge is an altered human IgG1 hinge in which the first cysteine is substituted by serine. The sequence of this altered IgG1 hinge is set forth in SEQ ID NO:664, and is referred to as the “human IgG1 SCC-P hinge” or “SCC-P hinge.” In certain embodiments, one or more amino acid residues (e.g., “RT,” “RSS,” or “T”) may be added at the amino- or carboxy-terminus of a mutated immunoglobulin hinge region as part of a fusion protein construct design.

In certain embodiments, a hinge polypeptide comprises or is a sequence that is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical to a wild type immunoglobulin hinge region, such as a wild type human IgG1 hinge, a wild type human IgG2 hinge, or a wild type human IgG4 hinge.

In further embodiments, a hinge present in a single chain polypeptide that forms a polypeptide heterodimer with another single chain polypeptide may be a hinge that is not based on or derived from an immunoglobulin hinge (i.e., not a wild type immunoglobulin hinge or an altered immunoglobulin hinge). These types of non-immunoglobulin based hinges may be used on or near the carboxyl end (e.g., located carboxyl terminal to Fc region portions) of the single chain polypeptides that form the polypeptide heterodimers. Examples for such hinges include peptides of about five to about 150 amino acids of the interdomain or stalk region of type II C-lectins or CD molecules, for instance, peptides of about eight to 25 amino acids and peptides of about seven to 18 amino acids, and derivatives thereof.

The “interdomain or stalk region” of a type II C-lectin or CD molecule refers to the portion of the extracellular domain of the type II C-lectin or CD molecule that is located between the C-type lectin-like domain (CTLD; e.g., similar to CTLD of natural killer cell receptors) and the transmembrane domain. For example, in the human CD94 molecule (GenBank Accession No. AAC50291.1, PRI Nov. 30, 1995), the extracellular domain corresponds to amino acid residues 34-179, whereas the CTLD corresponds to amino acid residues 61-176. Accordingly, the interdomain or stalk region of the human CD94 molecule includes amino acid residues 34-60, which is found between the membrane and the CTLD (see Boyington et al., Immunity 10:75, 1999; for descriptions of other stalk regions, see also Beavil et al., Proc. Nat'l. Acad. Sci. USA 89:753, 1992; and Figdor et al., Nature Rev. Immunol. 2:77, 2002). These type II C-lectin or CD molecules may also have from six to 10 junction amino acids between the stalk region and the transmembrane region or the CTLD. In another example, the 233 amino acid human NKG2A protein (GenBank Accession No. P26715.1, PRI Jun. 15, 2010) has a transmembrane domain ranging from amino acids 71-93 and an extracellular domain ranging from amino acids 94-233. The CTLD is comprised of amino acids 119-231, and the stalk region comprises amino acids 99-116, which is flanked by junctions of five and two amino acids. Other type II C-lectin or CD molecules, as well as their extracellular ligand-bind domains, interdomain or stalk regions, and CTLDs are known in the art (see, e.g., GenBank Accession Nos. NP_{—}001993.2; AAH07037.1, PRI Jul. 15, 2006; NP_{—}001773.1, PRI Jun. 20, 1010; AAL65234.1, PRI Jan. 17, 2002, and CAA04925.1, PRI Nov. 14, 2006, for the sequences of human CD23, CD69, CD72, NKG2A and NKG2D and their descriptions, respectively).

A “derivative” of an interdomain or stalk region, or fragment thereof, of a type II C-lectin or CD molecule includes an about an eight to about 150 amino acid sequence in which one, two, or three amino acids of the stalk region of a wild type type II C-lectin or CD molecule have a deletion, insertion, substitution, or any combination thereof, for instance, the one or more changes are substitutions or the one or more mutations include only one deletion. In further embodiments, a derivative of an interdomain or stalk region is more resistant to proteolytic cleavage as compared to the wild-type interdomain or stalk region sequence, such as those derived from about eight to about 20 amino acids of NKG2A, NKG2D, CD23, CD64, CD72, or CD94.

In certain embodiments, interdomain or stalk region hinges have seven to 18 amino acids and can form an α-helical coiled coil structure. In certain embodiments, interdomain or stalk region hinges contain 0, 1, 2, 3, or 4 cysteines. Exemplary interdomain or stalk region hinges are peptide fragments of the interdomain or stalk regions, such as ten to 150 amino acid fragments from the stalk regions of CD69, CD72, CD94, NKG2A and NKG2D, as set forth in SEQ ID NOS:241-244, 716 and 601. Additional exemplary stalk region or interdomain hinges include those as set forth in SEQ ID NOS:78, 734-737, 742-747, and 766-790.

Alternative hinges that can be used in single chain polypeptides of polypeptide heterodimers are from portions of cell surface receptors (interdomain regions) that connect immunoglobulin V-like or immunoglobulin C-like domains. Regions between Ig V-like domains where the cell surface receptor contains multiple Ig V-like domains in tandem and between Ig C-like domains where the cell surface receptor contains multiple tandem Ig C-like regions are also contemplated as hinges useful in single chain polypeptides of polypeptide heterodimers. In certain embodiments, hinge sequences comprised of cell surface receptor interdomain regions may further contain a naturally occurring or added motif, such as an IgG core hinge sequence that confers one or more disulfide bonds to stabilize the polypeptide heterodimer formation. Examples of hinges include interdomain regions between the Ig V-like and Ig C-like regions of CD2, CD4, CD22, CD33, CD48, CD58, CD66, CD80, CD86, CD150, CD166, and CD244.

In certain embodiments, hinge sequences have about 5 to 150 amino acids, 5 to 10 amino acids, 10 to 20 amino acids, 20 to 30 amino acids, 30 to 40 amino acids, 40 to 50 amino acids, 50 to 60 amino acids, 5 to 60 amino acids, 5 to 40 amino acids, 8 to 20 amino acids, or 10 to 15 amino acids. The hinge may be primarily flexible, but may also provide more rigid characteristics or may contain primarily α-helical structure with minimal β-sheet structure. The lengths or the sequences of the hinges may affect the binding affinities of the binding domains to which the hinges are directly or indirectly (via another region or domain, such as an immunoglobulin heterodimerization domain) connected as well as one or more activities of the Fc region portions to which the hinges are directly or indirectly connected (see, Examples 9 and 10).

In certain embodiments, hinge sequences are stable in plasma and serum and are resistant to proteolytic cleavage. The first lysine in the IgG1 upper hinge region may be mutated to minimize proteolytic cleavage, for instance, the lysine may be substituted with methionine, threonine, alanine or glycine, or is deleted (see, e.g., SEQ ID NOS:379-434, which may include junction amino acids at the amino terminus such as RT).

In some embodiments, hinge sequences may contain a naturally occurring or added motif such as an immunoglobulin hinge core structure CPPCP (SEQ ID NO:228) that confers the capacity to form a disulfide bond or multiple disulfide bonds to stabilize the carboxy-terminus of a molecule. In other embodiments, hinge sequences may contain one or more glycosylation sites.

Exemplary hinges, including altered immunoglobulin hinges, are set forth in SEQ ID NOS:379-434, 618-749, and 763-791.

In certain embodiments, a single chain polypeptide of a polypeptide heterodimer according to the present disclosure comprises more than one hinge. For example, a single chain polypeptide having two binding domains, one of which at the amino terminus and the other at the carboxyl terminus, may have two hinges. One hinge may be directly or indirectly (e.g., via an immunoglobulin heterodimerization domain) connected to the binding domain at or near the amino terminus, and the other hinge may be connected (e.g., directly connected) to the other binding domain at or near the carboxyl terminus. In certain embodiments, even if a single chain polypeptide has only one binding domain, it may have more than one hinge, for example, at its amino or carboxyl terminus. Such a hinge may interact with a corresponding hinge in the other chain of the heterodimer, such as forming one or more interchain disulfide bonds, to facilitate or enhance the heterodimerization of the two chains. A hinge (H-I) of a SCP-I of a polypeptide heterodimer “corresponds to” a hinge (H-II) of a SCP-II of the heterodimer when H-I and H-II are located on the same end of the Fc region portion of their respective single chain polypeptide. For example, a polypeptide heterodimer may comprise the following two single chain polypeptides: A first chain polypeptide from amino to carboxyl terminus comprises a first binding domain, CH1, hinge, CH2, and CH3, and a second chain polypeptide from amino to carboxyl terminus comprises a CK, first hinge, CH2, CH3, second hinge, and a second binding domain. The hinge in the first chain would be regarded as “corresponding” to the first hinge of the second chain because both are amino terminal to the Fc region portions to which they are connected.

In certain embodiments where a single chain polypeptide of a polypeptide heterodimer comprises a binding domain at or near its carboxyl terminus, a hinge may be present to link the binding domain with another portion of the single chain polypeptide (e.g., an Fc region portion or an immunoglobulin heterodimerization domain). In one embodiment, such a hinge is a non-immunoglobulin hinge (i.e., a hinge not based on or derived from a wild type immunoglobulin hinge) and may be a stalk region of a type II C-lectin or CD molecule, an interdomain region that connect IgV-like or IgC-like domains of a cell surface receptor, or a derivative or functional variant thereof. Exemplary carboxyl terminal hinges, sometimes referred to as “back-end” hinges, includes those set forth in SEQ ID NOS:78, 734-737, 742-747, and 766-790.

In certain embodiments, a hinge of one single chain polypeptide of a polypeptide heterodimer is identical to a corresponding hinge of the other single chain polypeptide of the heterodimer. In certain other embodiments, a hinge of one chain is different from that of the other chain (in their length or sequence). The different hinges in the different chains allow different manipulation of the binding affinities of the binding domains to which the hinges are connected, so that the heterodimer is able to preferentially bind to the target of one binding domain over the target of the other binding domain. For example, in certain embodiments, a polypeptide heterodimer has a CD3 or TCR binding domain in one chain and a tumor antigen binding domain in another chain. Having two different hinges in the two chains may allow the heterodimer to bind to the tumor antigen first, and then to a CD3 or TCR molecule second. Thus, the heterodimer may recruit CD3⁺ T cells to tumor cells carrying the tumor antigen, which in turn may damage or destroy the tumor cells.

Other Components or Modifications

In certain embodiments, a single chain polypeptide that forms a heterodimer with another single chain polypeptide may contain one or more additional domains or regions. Such additional regions may be a leader sequence (also referred to as “signal peptide”) at the amino-terminus for secretion of an expressed single chain polypeptide. Exemplary leader peptides of this disclosure include natural leader sequences or others, such as those as set forth in SEQ ID NOS:110 and 111.

Additional regions may also be sequences at the carboxy-terminus for identifying or purifying single chain polypeptides (e.g., epitope tags for detection or purification, such as a histidine tag, biotin, a FLAG® epitope, or any combination thereof).

Further optional regions may be additional amino acid residues (referred to as “junction amino acids” or “junction amino acid residues”) having a length of 1 to about 10 amino acids (e.g., about 2 to 5 amino acids), which may be resulted from use of specific expression systems or construct design for the single chain polypeptides of the present disclosure. Such additional amino acid residues (for instance, one, two, three, four or five additional amino acids) may be present at the amino or carboxyl terminus or between various regions or domains of a single chain polypeptide, such as between a binding domain and an immunoglobulin heterodimerization domain, between an immunoglobulin heterodimerization domain and a hinge, between a hinge and an Fc region portion, between domains of an Fc region portion (e.g., between CH2 and CH3 domains or between two CH3 domains), between a binding domain and a hinge, between an Fc region portion and an immunoglobulin heterodimerization domain, or between a variable domain and a linker. Exemplary junction amino acids amino-terminal to a hinge include RDQ (SEQ ID NO:598), RT, SS, SASS (SEQ ID NO:599) and SSS (SEQ ID NO:600). Exemplary junction amino acids carboxy-terminal to a hinge include amino acids SG. Additional exemplary junction amino acids include SR.

In certain embodiments, junction amino acids are present between an Fc region portion that comprises CH2 and CH3 domains and an immunoglobulin heterodimerization domain (CH1 or CL). These junction amino acids are also referred to as a “linker between CH3 and CH1 or CL” if they are present between the C-terminus of CH3 and the N-terminus of CH1 or CL. Such a linker may be about 2-12 amino acids in length. In certain embodiments, the Fc region portion comprises human IgG1 CH2 and CH3 domains in which the C-terminal lysine residue of human IgG1 CH3 is deleted. Exemplary linkers between CH3 and CH1 include those set forth in SEQ ID NO:847-849. Exemplary linkers between CH3 and Ck include those set forth in SEQ ID NOS:850-852 (in which the carboxyl terminal arginine in the linkers may alternatively be regarded as the first arginine of Ck). In certain embodiments, the presence of such linkers or linker pairs (e.g., SEQ ID NO:847 as a CH3-CH1 linker in one single chain polypeptide of a heterodimer and SEQ ID NO:850 as a CH3-Ck linker in the other single chain polypeptide of the heterodimer; SEQ ID NO:848 as a CH3-CH1 linker and SEQ ID NO:851 as a CH3-Ck linker; and SEQ ID NO:849 as a CH3-CH1 linker and SEQ ID NO:852 as a CH3-Ck linker) improves the production of heterodimer compared the presence of a reference linker as set forth in SEQ ID NO:846 (in which the last lysine of CH3 is included as part of the linker) in both single chain polypeptides of a heterodimer.

In certain embodiments, an immunoglobulin Fc region (e.g., CH2, CH3, and/or CH4 regions) of a polypeptide heterodimer of the present disclosure may have an altered glycosylation pattern relative to an immunoglobulin reference sequence. For example, any of a variety of genetic techniques may be employed to alter one or more particular amino acid residues that form a glycosylation site (see Co et al. (1993) Mol. Immunol. 30:1361; Jacquemon et al. (2006) J. Thromb. Haemost. 4:1047; Schuster et al. (2005) Cancer Res. 65:7934; Warnock et al. (2005) Biotechnol. Bioeng. 92:831), such as N297 of the CH2 domain (EU numbering). Alternatively, the host cells producing polypeptide heterodimers of this disclosure may be engineered to produce an altered glycosylation pattern. One method known in the art, for example, provides altered glycosylation in the form of bisected, non-fucosylated variants that increase ADCC. The variants result from expression in a host cell containing an oligosaccharide-modifying enzyme. Alternatively, the POTELLIGENT® technology of BioWa/Kyowa Hakko is contemplated to reduce the fucose content of glycosylated molecules according to this disclosure. In one known method, a CHO host cell for recombinant immunoglobulin production is provided that modifies the glycosylation pattern of the immunoglobulin Fc region, through production of GDP-fucose.

Alternatively, chemical techniques are used to alter the glycosylation pattern of polypeptide heterodimers of this disclosure. For example, a variety of glycosidase and/or mannosidase inhibitors provide one or more of desired effects of increasing ADCC activity, increasing Fc receptor binding, and altering glycosylation pattern. In certain embodiments, cells expressing polypeptide heterodimers of the instant disclosure are grown in a culture medium comprising a carbohydrate modifier at a concentration that increases the ADCC of immunoglycoprotein molecules produced by said host cell, wherein said carbohydrate modifier is at a concentration of less than 800 μM. In one embodiment, the cells expressing these polypeptide heterodimers are grown in a culture medium comprising castanospermine or kifunensine, for instance, castanospermine at a concentration of 100-800 μM, such as 100 μM, 200 μM, 300 μM, 400 μM, 500 μM, 600 μM, 700 μM, or 800 μM. Methods for altering glycosylation with a carbohydrate modifier such as castanospermine are provided in U.S. Pat. No. 7,846,434 or PCT Publication No. WO 2008/052030.

Structural Arrangements and Exemplary Heterodimers

To form a polypeptide heterodimer according to the present invention, two single chain polypeptides are designed so that the immunoglobulin heterodimerization domain of the first single chain polypeptide is properly aligned and interacts with the immunoglobulin heterodimerization domain of the second single chain polypeptide. In certain embodiments, the heterodimer may comprise a second immunoglobulin heterodimerization domain pair to facilitate or enhance the heterodimerization of the two chains. In certain embodiments, in addition to the interaction between the two immunoglobulin heterodimerization domains, an Fc region portion (e.g., a CH3 domain) in the first chain may interact with an Fc region in the second chain to enhance heterodimerization (e.g., via interaction between two wild type CH3 domains or between a CH3 domain pair with “knobs-into-holes” mutations). Moreover, in certain embodiments, the hinge in the first chain (e.g., an altered human IgG1 hinge with two cysteine residues as set forth in SEQ ID NO:664) may interact with the hinge in the second chain (e.g., the same altered human IgG1 hinge as set forth in SEQ ID NO:664) to form, for example, disulfide bonds, which may further strengthen the interaction between the first and second single chain polypeptides to form a polypeptide heterodimer of the present disclosure. Furthermore, in certain embodiments, the first and second chain may comprise a second hinge pair (e.g., at the carboxyl termini of the two chains) to further enhance the interactions between the two chains.

In certain embodiments, a polypeptide heterodimer of the present disclosure comprises two binding domains (BD1 and BD2), wherein the binding domains bind to two different target molecules. In certain embodiments, the two binding domains (BD1 and BD2) are both on the SCP-I with the immunoglobulin heterodimerization domain (HD-I) and the Fc region portion (FRP-1) disposed between BD1 and BD2. In certain other embodiments, the first binding domain (BD1) is on the SCP-I and the second binding domain (BD2) is on the SCP-II. In certain embodiments, both BD1 and BD2 are amino terminal to the Fc region portion of the SCP-I and SCP-II, respectively. In certain other embodiments, BD1 is amino terminal to the Fc region portion of the SCP-I and BD2 is carboxyl terminal to the Fc region portion of the SCP-II. In certain other embodiments, BD1 is carboxyl terminal to the Fc region portion of the SCP-I and BD2 is amino terminal to the Fc region portion of the SCP-II. In certain other embodiments, both BD1 and BD2 are carboxyl terminal to the Fc region portion of the SCP-I and SCP-II, respectively.

In certain embodiments, a polypeptide heterodimer comprises three binding domains (BD1, BD2 and BD3), wherein the binding domains bind to two or three different target molecules. For example, BD1, BD2, and BD3 each bind a different target, or BD1 and BD2 bind a first target while BD3 binds a second target, or BD1 and BD3 bind a first target while BD2 binds a second target, or BD2 and BD3 bind a first target while BD1 binds a second target. In certain embodiments, the immunoglobulin heterodimerization domain and the Fc region portion are disposed between BD1 and BD2 on SCP-I, and BD3 is amino terminal to the Fc region portion of the SCP-II. In further embodiments, the immunoglobulin heterodimerization domain and the Fc region portion are disposed between BD1 and BD2 on SCP-II, and BD3 is carboxyl terminal to the Fc region portion of the SCP-I.

In certain embodiments, a polypeptide heterodimer comprises four binding domains (BD1, BD2, BD3 and BD4), wherein the binding domains bind to two to four different target molecules. In certain embodiments, the immunoglobulin heterodimerization domain and Fc region portion of the SCP-I are disposed between BD1 and BD2, and the immunoglobulin heterodimerization domain and Fc region portion of SCP-II are disposed between BD3 and BD4.

In certain embodiments, a polypeptide heterodimer comprises five binding domains (BD1, BD2, BD3, BD4 and BD5), wherein the binding domains bind to two to four different target molecules. In certain embodiments, SCP-I comprises three binding domains (BD1, BD2 and BD3), and SCP-II comprises two binding domains (BD4 and BD5). In further embodiments, BD1 and BD2 may be, for example, linked in tandem to each other (either directly or via a peptide linker of about two to eight amino acids) at the amino (or carboxyl) terminus of SCP-I with BD3 located at the carboxyl (or amino) terminus of SCP-I or SCP-II, wherein BD4 and BD5 are on SCP-II if BD3 is on SCP-I, or BD4 or BD5 are on SCP-I if BD3 is on SCP-II.

In still further embodiments, the polypeptide heterodimer comprises six binding domains (BD1-BD6). In certain embodiments, SCP-I and SCP-II each comprise three binding domains (e.g., BD1-BD3 on SCP-I, and BD4-BD6 on SCP-II). In such embodiments, for example, BD1 and BD2 may be linked in tandem and located at the SCP-I amino (or carboxyl) terminus, and BD3 may be located at the SCP-I carboxyl (or amino) terminus. Similarly, BD4 and BD5 may be linked in tandem and located at the SCP-II amino (or carboxyl) terminus, and BD6 may be located at the SCP-II carboxyl (or amino) terminus. In certain other embodiments, the first single chain polypeptide (SCP-I) comprises four binding domains (BD1-BD4) and the second single chain polypeptide (SCP-II) comprises two binding domains (BD5 and BD6). In such embodiments, BD1 and BD2 may be linked in tandem and located at or near amino terminus of SCP-I, BD3 and BD4 may be linked in tandem and located at or near carboxyl terminus of SCP-I, and BD5 and BD6 may be located at or near the amino and carboxyl termini of SCP-II, respectively.

In certain embodiments, the polypeptide heterodimer comprises seven binding domains (BD1-BD7). In such embodiments, SCP-I may comprise four binding domains (BD1-BD4), and SCP-II may comprise the other three binding domains (BD5-BD7). For example, BD1 and BD2 may be linked in tandem and located at or near the amino terminus of SCP-I, and BD3 and BD4 may be linked in tandem and located at or near the carboxyl terminus of SCP-II. BD5 and BD6 may be linked in tandem and located at or near the amino (or carboxyl) terminus of SCP-II, and BD7 may be located at or near the carboxyl (or amino) terminus of SCP-II.

In certain embodiments, the polypeptide heterodimer comprises eight binding domains (BD1-BD8). In such embodiments, the first and second single chain polypeptides may each comprise four binding domains. In each chain, two binding domains may be located at or near the amino terminus, and the other two binding domains located at or near the carboxyl terminus.

To simplify the description of how various components can be arranged to make first and second single chain polypeptides that form polypeptide heterodimers of the present disclosure, arrangements in exemplary embodiments (1) to (50) are provided below in which only two binding domains are included in each heterodimer.

In embodiment (1), a polypeptide heterodimer comprises the following two single chain polypeptides: from amino terminus to carboxyl terminus, a first single chain polypeptide comprising a first binding domain, a CH1 region, a hinge, and an Fc region portion; and a second single chain polypeptide comprising a second binding domain, a CL region (e.g., Cκ, Cλ), a hinge, and an Fc region portion.

In embodiment (2), a polypeptide heterodimer comprises the following two single chain polypeptides: from amino terminus to carboxyl terminus, a first single chain polypeptide comprising a first binding domain, a hinge, an Fc region portion, and a CH1 region; and a second single chain polypeptide comprising a second binding domain, a hinge, an Fc region portion, and a CL region.

In embodiment (3), a polypeptide heterodimer comprises the following two single chain polypeptides: from amino terminus to carboxyl terminus, a first single chain polypeptide comprising a first binding domain, a CH1 region, a hinge, an Fc region portion, and a second CH1 region; and a second single chain polypeptide comprising a second binding domain, a CL region, a hinge, an Fc region portion, and a second CL region.

In embodiment (4), a polypeptide heterodimer comprises the following two single chain polypeptides: from amino terminus to carboxyl terminus, a first single chain polypeptide comprising a first binding domain, a CH1 region, a second CH1 region, a hinge, and an Fc region portion; and a second single chain polypeptide comprising a second binding domain, a CL region, a second CL region, a hinge and an Fc region portion.

In embodiment (5), a polypeptide heterodimer comprises the following two single chain polypeptides: from amino terminus to carboxyl terminus, a first single chain polypeptide comprising a first binding domain, a hinge, an Fc region portion, a CH1 region, and a second CH1 region; and a second single chain polypeptide comprising a second binding domain, a hinge, an Fc region portion, a CL region and a second CL region.

In embodiment (6), a polypeptide heterodimer comprises the following two single chain polypeptides: from amino terminus to carboxyl terminus, a first single chain polypeptide comprising a CH1 region, a hinge, an Fc region portion, a second hinge, and a first binding domain; and a second single chain polypeptide comprising a CL region, a hinge, an Fc region portion, a second hinge, and a second binding domain.

In embodiment (7), a polypeptide heterodimer is formed from the following two single chain polypeptides: from amino terminus to carboxyl terminus, a first single chain polypeptide comprising a hinge, an Fc region portion, a CH1 region, a second hinge, and a first binding domain; and a second single chain polypeptide comprising a hinge, an Fc region portion, a CL region, a second hinge, and a second binding domain.

In embodiment (8), a polypeptide heterodimer comprises the following two single chain polypeptides: from amino terminus to carboxyl terminus, a first single chain polypeptide comprising a CH1 region, a hinge, an Fc region portion, a second CH1 region, a second hinge, and a first binding domain; and a second single chain polypeptide comprising a CL region, a hinge, an Fc region portion, a second CL region, a second hinge, and a second binding domain.

In embodiment (9), a polypeptide heterodimer comprises the following two single chain polypeptides: from amino terminus to carboxyl terminus, a first single chain polypeptide comprising a CH1 region, a second CH1 region, a hinge, an Fc region portion, a second hinge, and a first binding domain; and a second single chain polypeptide comprising a CL region, a second CL region, a hinge, an Fc region portion, a second hinge, and a second binding domain.

In embodiment (10), a polypeptide heterodimer comprises the following two single chain polypeptides: from amino terminus to carboxyl terminus, a first single chain polypeptide comprising a hinge, an Fc region portion, a CH1 region, a second CH1 region, a second hinge, and a first binding domain; and a second single chain polypeptide comprising a hinge, an Fc region portion, a CL region, a second CL region, a second hinge, and a second binding domain.

In embodiment (11), a polypeptide heterodimer comprises the following two single chain polypeptides: from amino terminus to carboxyl terminus, a first single chain polypeptide comprising a first binding domain, a CL region, a hinge, and an Fc region portion; and a second single chain polypeptide comprising a second binding domain, a CH1 region, a hinge, and an Fc region portion.

In embodiment (12), a polypeptide heterodimer comprises the following two single chain polypeptides: from amino terminus to carboxyl terminus, a first single chain polypeptide comprising a first binding domain, a hinge, an Fc region portion, and a CL region; and a second single chain polypeptide comprising a second binding domain, a hinge, an Fc region portion, and a CH1 region.

In embodiment (13), a polypeptide heterodimer comprises the following two single chain polypeptides: from amino terminus to carboxyl terminus, a first single chain polypeptide comprising a first binding domain, a CL region, a hinge, an Fc region portion, and a second CL region; and a second single chain polypeptide comprising a second binding domain, a CH1 region, a hinge, an Fc region portion, and a second CH1 region.

In embodiment (14), a polypeptide heterodimer comprises the following two single chain polypeptides: from amino terminus to carboxyl terminus, a first single chain polypeptide comprising a first binding domain, a CL region, a second CL region, a hinge, and an Fc region portion; and a second single chain polypeptide comprising a second binding domain, a CH1 region, a second CH1 region, a hinge, and an Fc region portion.

In embodiment (15), a polypeptide heterodimer comprises the following two single chain polypeptides: from amino terminus to carboxyl terminus, a first single chain polypeptide comprising a first binding domain, a hinge, an Fc region portion, a CL region, and a second CL region; and a second single chain polypeptide comprising a second binding domain, a hinge, an Fc region portion, a CH1 region, and a second CH1 region.

In embodiment (16), a polypeptide heterodimer comprises the following two single chain polypeptides: from amino terminus to carboxyl terminus, a first single chain polypeptide comprising a CL region, a hinge, an Fc region portion, a second hinge, and a first binding domain; and a second single chain polypeptide comprising a CH1 region, a hinge, an Fc region portion, a second hinge, and a second binding domain.

In embodiment (17), a polypeptide heterodimer comprises the following two single chain polypeptides: from amino terminus to carboxyl terminus, a first single chain polypeptide comprising a hinge, an Fc region portion, a CL region, a second hinge, and a first binding domain; and a second single chain polypeptide comprising a hinge, an Fc region portion, a CH1 region, a second hinge, and a second binding domain.

In embodiment (18), a polypeptide heterodimer comprises the following two single chain polypeptides: from amino terminus to carboxyl terminus, a first single chain polypeptide comprising a CL region, a hinge, an Fc region portion, a second CL region, a second hinge, and a first binding domain; and a second single chain polypeptide comprising a CH1 region, a hinge, an Fc region portion, a second CH1 region, a second hinge, and a second binding domain.

In embodiment (19), a polypeptide heterodimer comprises the following two single chain polypeptides: from amino terminus to carboxyl terminus, a first single chain polypeptide comprising a CL region, a second CL region, a hinge, an Fc region portion, a second hinge, and a first binding domain; and a second single chain polypeptide comprising a CH1 region, a second CH1 region, a hinge, an Fc region portion, a second hinge, and a second binding domain.

In embodiment (20), a polypeptide heterodimer comprises the following two single chain polypeptides: from amino terminus to carboxyl terminus, a first single chain polypeptide comprising a hinge, an Fc region portion, a CL region, a second CL region, a second hinge, and a first binding domain; and a second single chain polypeptide comprising a hinge, an Fc region portion, a CH1 region, a second CH1 region, a second hinge, and a second binding domain.

In embodiment (21), a polypeptide heterodimer comprises the following two single chain polypeptides: from amino terminus to carboxyl terminus, a first single chain polypeptide comprising a first binding domain, a CH1 region, a hinge, an Fc region portion, and a CL region; and a second single chain polypeptide comprising a second binding domain, a CL region, a hinge, an Fc region portion, and a CH1 region.

In embodiment (22), a polypeptide heterodimer comprises the following two single chain polypeptides: from amino terminus to carboxyl terminus, a first single chain polypeptide comprising a first binding domain, a CL region, a hinge, an Fc region portion, and a CH1 region; and a second single chain polypeptide comprising a second binding domain, a CH1 region, a hinge, an Fc region portion, and a CL region.

In embodiment (23), a polypeptide heterodimer comprises the following two single chain polypeptides: from amino terminus to carboxyl terminus, a first single chain polypeptide comprising a first binding domain, a CH1 region, a CL region, a hinge, and an Fc region portion; and a second single chain polypeptide comprising a second binding domain, a CL region, a CH1 region, a hinge and an Fc region portion.

In embodiment (24), a polypeptide heterodimer comprises the following two single chain polypeptides: from amino terminus to carboxyl terminus, a first single chain polypeptide comprising a first binding domain, a CL region, a CH1 region, a hinge, and an Fc region portion; and a second single chain polypeptide comprising a second binding domain, a CH1 region, a CL region, a hinge and an Fc region portion.

In embodiment (25), a polypeptide heterodimer comprises the following two single chain polypeptides: from amino terminus to carboxyl terminus, a first single chain polypeptide comprising a first binding domain, a hinge, an Fc region portion, a CH1 region and a CL region; and a second single chain polypeptide comprising a second binding domain, a hinge, an Fc region portion, a CL region and a CH1 region.

In embodiment (26), a polypeptide heterodimer comprises the following two single chain polypeptides: from amino terminus to carboxyl terminus, a first single chain polypeptide comprising a first binding domain, a hinge, an Fc region portion, a CL region and a CH1 region; and a second single chain polypeptide comprising a second binding domain, a hinge, an Fc region portion, a CH1 region and a CL region.

In embodiment (27), a polypeptide heterodimer comprises the following two single chain polypeptides: from amino terminus to carboxyl terminus, a first single chain polypeptide comprising a CH1 region, a hinge, an Fc region portion, a CL region, a second hinge, and a first binding domain; and a second single chain polypeptide comprising a CL region, a hinge, an Fc region portion, a CH1 region, a second hinge, and a second binding domain.

In embodiment (28), a polypeptide heterodimer comprises the following two single chain polypeptides: from amino terminus to carboxyl terminus, a first single chain polypeptide comprising a CL region, a hinge, an Fc region portion, a CH1 region, a second hinge, and a first binding domain; and a second single chain polypeptide comprising a CH1 region, a hinge, an Fc region portion, a CL region, a second hinge, and a second binding domain.

In embodiment (29), a polypeptide heterodimer comprises the following two single chain polypeptides: from amino terminus to carboxyl terminus, a first single chain polypeptide comprising a CH1 region, a CL region, a hinge, an Fc region portion, a second hinge, and a first binding domain; and a second single chain polypeptide comprising a CL region, a CH1 region, a hinge, an Fc region portion, a second hinge, and a second binding domain.

In embodiment (30), a polypeptide heterodimer comprises the following two single chain polypeptides: from amino terminus to carboxyl terminus, a first single chain polypeptide comprising a CL region, a CH1 region, a hinge, an Fc region portion, a second hinge, and a first binding domain; and a second single chain polypeptide comprising a CH1 region, a CL region, a hinge, an Fc region portion, a second hinge, and a second binding domain.

In embodiment (31), a polypeptide heterodimer comprises the following two single chain polypeptides: from amino terminus to carboxyl terminus, a first single chain polypeptide comprising a hinge, an Fc region portion, a CH1 region, a CL region, a second hinge, and a first binding domain; and a second single chain polypeptide comprising a hinge, an Fc region portion, a CL region, a CH1 region, a second hinge, and a second binding domain.

In embodiment (32), a polypeptide heterodimer comprises the following two single chain polypeptides: from amino terminus to carboxyl terminus, a first single chain polypeptide comprising a hinge, an Fc region portion, a CL region, a CH1 region, a second hinge, and a first binding domain; and a second single chain polypeptide comprising a hinge, an Fc region portion, a CH1 region, a CL region, a second hinge, and a second binding domain.

In embodiment (33), a polypeptide heterodimer comprises the following two single chain polypeptides: from amino to carboxyl terminus, a first single chain polypeptide comprising a first binding domain, a CH1 region, a hinge, an Fc region portion, a second hinge, and a second binding domain; and a second single chain polypeptide comprising a CL region, a hinge, and an Fc region.

In embodiment (34), a polypeptide heterodimer comprises the following two single chain polypeptides: from amino to carboxyl terminus, a first single chain polypeptide comprising a first binding domain, a CL region, a hinge, an Fc region portion, a second hinge, and a second binding domain; and a second single chain polypeptide comprising a CH1 region, a hinge and an Fc region portion.

In embodiment (35), a polypeptide heterodimer comprises the following two single chain polypeptides: from amino to carboxyl terminus, a first single chain polypeptide comprising a first binding domain, a CH1 region, a hinge and an Fc region portion; and a second single chain polypeptide comprising a CL region, a hinge, an Fc region portion, a second hinge and a second binding domain.

In embodiment (36), a polypeptide heterodimer comprises the following two single chain polypeptides: from amino to carboxyl terminus, a first single chain polypeptide comprising a CH1 region, a hinge, an Fc region portion, a second hinge and a first binding domain; and a second single chain polypeptide comprising a second binding domain, a CL region, a hinge and an Fc region portion.

In embodiment (37), a polypeptide heterodimer comprises the following two single chain polypeptides: from amino to carboxyl terminus, a first single chain polypeptide comprising a first binding domain, a CH1 region, a hinge, an Fc region portion, and a second hinge; and a second single chain polypeptide comprising a second binding domain, a CL region, a hinge, an Fc region portion, and a second hinge.

In embodiment (38), a polypeptide heterodimer comprises the following two single chain polypeptides: from amino to carboxyl terminus, a first single chain polypeptide comprising a first binding domain, a hinge, an Fc region portion, a CH1 region, a second hinge, and a second binding domain; and the second single chain polypeptide comprising a hinge, an Fc region portion, and a CL region.

In embodiment (39), a polypeptide heterodimer comprises the following two single chain polypeptides: from amino to carboxyl terminus, a first single chain polypeptide comprising a hinge, an Fc region portion, and a CH1 region; and a second single chain polypeptide comprising a first binding domain, a hinge, an Fc region portion, a CL region, a second hinge, and a second binding domain.

In embodiment (40), a polypeptide heterodimer comprises the following two single chain polypeptides: from amino to carboxyl terminus, a first single chain polypeptide comprising a first binding domain, a hinge, an Fc region portion, and a CH1 region; and a second single chain polypeptide comprising a hinge, an Fc region portion, a CL region, a second hinge and a second binding domain.

In embodiment (41), a polypeptide heterodimer comprises the following two single chain polypeptides: from amino to carboxyl terminus, a first single chain polypeptide comprising a hinge, an Fc region portion, a CH1 region, a second hinge and a first binding domain; and the second single chain polypeptide comprising a second binding domain, a hinge, an Fc region portion, and a CL region.

In embodiment (42), a polypeptide heterodimer comprises the following two single chain polypeptides: from amino to carboxyl terminus, a first single chain polypeptide comprising a first binding domain, a hinge, an Fc region portion, a CH1 region, and a second hinge; and a second single chain polypeptide comprising a second binding domain, a hinge, an Fc region portion, a CL region and a second hinge.

In embodiment (43), a polypeptide heterodimer comprises the following two single chain polypeptides: from amino to carboxyl terminus, a first single chain polypeptide comprising a first binding domain, a CH1 region, a hinge an Fc region portion, a CL region, a second hinge and a second binding domain; and the second single chain polypeptide comprising a CL region, a hinge, an Fc region portion and a CH1 region.

In embodiment (44), a polypeptide heterodimer comprises the following two single chain polypeptides: from amino to carboxyl terminus, a first single chain polypeptide comprising a CH1 region, a hinge, an Fc region portion, and a CL region; and a second single chain polypeptide comprising a first binding domain, a CL region, a hinge, an Fc region, a CH1 region, a second hinge and a second binding domain.

In embodiment (45), a polypeptide heterodimer comprises the following two single chain polypeptides: from amino to carboxyl terminus, a first single chain polypeptide comprising a first binding domain, a CH1 region, a hinge, an Fc region portion, and a CL region; and a second single chain polypeptide comprising a CL region, a hinge, an Fc region portion, a CH1 region, a second hinge, and a second binding domain.

In embodiment (46), a polypeptide heterodimer comprises the following two single chain polypeptides: from amino to carboxyl terminus, a first single chain polypeptide comprising a CH1 region, a hinge, an Fc region portion, a CL, a second hinge and a first binding domain; and a second single chain polypeptide comprising a second binding domain, a CL region, a hinge, an Fc region portion, and a CH1 region.

In embodiment (47), a polypeptide heterodimer comprises the following two single chain polypeptides: from amino to carboxyl terminus, a first single chain polypeptide comprising a first binding domain, a CH1 region, a hinge, an Fc region portion, a second CH1 region, a second hinge, and a second binding domain; and a second single chain polypeptide comprising a CL region, a hinge, an Fc region portion, and a second CL region.

In embodiment (48), a polypeptide heterodimer comprises the following two single chain polypeptides: from amino to carboxyl terminus, a first single chain polypeptide comprising a CH1 region, a hinge, an Fc region portion, and a second CH1 region; and a second single chain polypeptide comprising a first binding domain, a CL region, a hinge, an Fc region portion, a second CL region, a second hinge, and a second binding domain.

In embodiment (49), a polypeptide heterodimer comprises the following two single chain polypeptides: from amino to carboxyl terminus, a first single chain polypeptide comprising a first binding domain, a CH1 region, a hinge, an Fc region portion, and a second CH1 region; and a second single chain polypeptide comprising a CL region, a hinge, an Fc region portion, a second CL region, a second hinge and a second binding domain.

In embodiment (50), a polypeptide heterodimer comprises the following two single chain polypeptides: from amino to carboxyl terminus, a first single chain polypeptide comprising a CH1 region, a hinge, an Fc region portion, a CH1 region, a second hinge, and a first binding domain; and a second single chain polypeptide comprising a second binding domain, a CL region, a hinge, an Fc region portion, and a second CL region.

Exemplary embodiments (51) to (60) are also provided below in which three or four binding domains are included in each heterodimer. Additional binding domains may be included to make polypeptide heterodimers that comprise five to eight binding domains according to the present disclosure in view of the general description provided herein.

In embodiment (51), a polypeptide heterodimer comprises the following two single chain polypeptides: from amino to carboxyl terminus, a first single chain polypeptide comprising a first binding domain, a CH1 region, a hinge, an Fc region portion, a second hinge and a second binding domain; and a second single chain polypeptide comprising a third binding domain, a CL region, a hinge and an Fc region portion.

In embodiment (52), a polypeptide heterodimer comprises the following two single chain polypeptides: from amino to carboxyl terminus, a first single chain polypeptide comprising a first binding domain, a CH1 region, a hinge, an Fc region portion, a second hinge, and a second binding domain; and a second single chain polypeptide comprising a CL region, a hinge, an Fc region portion, a second hinge and a third binding domain.

In embodiment (53), a polypeptide heterodimer comprises the following two single chain polypeptides: from amino to carboxyl terminus, a first single chain polypeptide comprising a first binding domain, a CL region, a hinge, an Fc region portion, a second hinge and a second binding domain; and the second single chain polypeptide comprising a third binding domain, a CH1 region, a hinge and an Fc region portion.

In embodiment (54), a polypeptide heterodimer comprises the following two single chain polypeptides: from amino to carboxyl terminus, a first single chain polypeptide comprising a first binding domain, a CL region, a hinge, an Fc region portion, a second hinge and a second binding domain; and a second single chain polypeptide comprising a CH1 region, a hinge, an Fc region portion, a second hinge and a third binding domain.

In embodiment (55), a polypeptide heterodimer comprises the following two single chain polypeptides: from amino to carboxyl terminus, a first single chain polypeptide comprising a first binding domain, a hinge, an Fc region portion, a CH1 region, a second hinge and a second binding domain; and the second single chain polypeptide comprising a third binding domain, a hinge, an Fc region portion and a CL region.

In embodiment (56), a polypeptide heterodimer comprises the following two single chain polypeptides: from amino to carboxyl terminus, a first single chain polypeptide comprising a first binding domain, a hinge, an Fc region portion, a CH1 region, a second hinge, and a second binding domain; and a second single chain polypeptide comprising a hinge, an Fc region portion, a CL region, a second hinge, and a third binding domain.

In embodiment (57), a polypeptide heterodimer comprises the following two single chain polypeptides: from amino to carboxyl terminus, a first single chain polypeptide comprising a first binding domain, a hinge, an Fc region portion, a CL region, a second hinge, and a second binding domain; and a second single chain polypeptide comprising a third binding domain, a hinge, an Fc region portion and a CH1 region.

In embodiment (58), a polypeptide heterodimer comprises the following two single chain polypeptides: from amino to carboxyl terminus, a first single chain polypeptide comprising a first binding domain, a hinge, an Fc region portion, a CL region, a second hinge, and a second binding domain; and a second single chain polypeptide comprising a hinge, an Fc region portion, a CH1 region, a second hinge, and a third binding domain.

In embodiment (59), a polypeptide heterodimer comprises the following two single chain polypeptides: from amino to carboxyl terminus: a first binding domain, a CH1 region, a hinge, an Fc region portion, a second hinge, and a second binding domain; and a second single chain polypeptide comprising a third binding domain, a CL region, a hinge, an Fc region portion, a second hinge and a fourth binding domain.

In embodiment (60), a polypeptide heterodimer comprises the following two single chain polypeptides: from amino to carboxyl terminus, a first single chain polypeptide comprising a first binding domain, a hinge, an Fc region portion, a CH1 region, a second hinge and a second binding domain; and a second single chain polypeptide comprising a third binding domain, a hinge, an Fc region portion, a CL region, a second hinge and a fourth binding domain.

In embodiments (1) to (32), a polypeptide heterodimer of the present disclosure comprises the following two single chain polypeptides: a first single chain polypeptide comprising a first binding domain (BD1) that specifically binds to a T cell target (e.g., a TCR complex or a component thereof, including TCRα, TCRβ, CD3γ, CD3δ, and CD3ε), a hinge that is an SCC-P IgG1 hinge, an Fc region portion that is a wild type or altered human IgG1 CH2CH3, and a CL region that is wild type or an altered human Ck region with N30Y V55A T70E (YAE) substitutions; and a second single chain polypeptide comprising binding domain (BD2) that specifically binds to a B-cell target (e.g., CD19, CD79b, HLA-DR, CD37, CD20) or a tumor or cancer antigen (e.g., RON, c-Met, EpCAM, CEACAM-6, PSMA), a hinge that is also an SCC-P IgG1 hinge, an Fc region portion that is a wild type or altered human IgG1 CH2CH3, and a CH1 region that is human CH1 region.

In further embodiments, a polypeptide heterodimer of embodiments (1) to (32) may further comprise a third binding domain (BD3) that is identical to BD1 or BD2, and is linked to a single chain polypeptide via a second hinge (e.g., linker H75 or H68 as set forth in SEQ ID NOS:742 and 78, respectively). In still further embodiments, a polypeptide heterodimer of embodiment of embodiments (1) to (32) may further comprise third and fourth binding domains (BD3) and (BD4) that each are identical or different to BD1 or BD2 and linked to the single chain polypeptide(s) via a second hinge (e.g., linker H75 or H68 as set forth in SEQ ID NOS:742 and 78).

In certain embodiments, the polypeptide heterodimers of the present disclosure can be engineered to have binding domains with different affinities to target specific cell types. For example, it may be desirable for a polypeptide heterodimer with a binding domain for a TCR complex or a component thereof and another binding domain for a tumor antigen (or a B-cell target) to preferentially bind tumor cells having the tumor antigen (or B-cells carrying the B-cell target) with a higher affinity so that the polypeptide heterodimer will bind the tumor cells (or the B-cells) first and then recruit T cells via its TCR/CD3 binding domain to the tumor site or cells. Differential binding affinities may be achieved by, for example, choosing a binding domain for one target with a higher binding affinity than the other binding domain has for its target or by including multiple binding domains for one target on a polypeptide heterodimer and a single binding domain or fewer binding domains for the second or other targets. In addition, using different hinges (e.g., using hinges of different lengths) may affect binding of one domain more than another or using different hinges for different binding domains in order to alter the binding activity of the binding domains.

In certain embodiments, multiple binding domains may need to be located at an appropriate distance from each other so that their interactions with their targets will produce a desirable effect. For example, in certain embodiments, a polypeptide heterodimer comprising a binding domain for a TCR complex or a component thereof in a single chain polypeptide and another binding domain for a tumor antigen or a B-cell target in a second single chain polypeptide may have both binding domains at the amino or carboxyl termini of their corresponding chains so that they are in physical proximity to each other in the polypeptide heterodimer.

Exemplary heterodimers may be formed from single chain polypeptide pairs described herein. If sequence identification numbers noted herein contain signal peptide sequences (e.g., the first 20 amino acids), such signal peptide sequences are not part of the mature single chain polypeptides that form the exemplary polypeptide heterodimers and thus should be considered excluded.

Exemplary single chain polypeptides are set forth in SEQ ID NOS:2, 4, 6, 8, 10, 12, 14, 16, 18, 20, 22, 24, 29-32, 53-72, 74, 810-826, 859-864, and 874-882.

Exemplary heterodimers may be formed from the following single chain polypeptide pairs: SEQ ID NOS:2 and 4, SEQ ID NOS:6 and 8, SEQ ID NOS:10 and 12, SEQ ID NOS:14 and 16, SEQ ID NOS:18 and 20, SEQ ID NOS:20 and 22, SEQ ID NOS:20 and 24, SEQ ID NOS:30 and 32, SEQ ID NOS:29 and 31, SEQ ID NOS:29 and 32, SEQ ID NOS:30 and 72, SEQ ID NOS:53 and 72, SEQ ID NOS:54 and 72, SEQ ID NOS:55 and 72, SEQ ID NOS:70 and 72, SEQ ID NOS:71 and 72, SEQ ID NOS:63 and 56, SEQ ID NOS:64 and 57, SEQ ID NOS:65 and 60, SEQ ID NOS:66 and 58, SEQ ID NOS:67 and 59, SEQ ID NOS:68 and 61, SEQ ID NOS:69 and 62, SEQ ID NOS:54 and 811, SEQ ID NOS:54 and 812, SEQ ID NOS:54 and 813, SEQ ID NOS:814 and 818, SEQ ID NOS:815 and 818, SEQ ID NOS:816 and 818, SEQ ID NOS:817 and 818, SEQ ID NOS:814 and 820, SEQ ID NOS:814 and 821, SEQ ID NOS:54 and 819, SEQ ID NOS:814 and 826, SEQ ID NOS:814 and 822, SEQ ID NOS:814 and 823, SEQ ID NOS:814 and 824, SEQ ID NOS:859 and 862, SEQ ID NOS:860 and 863, SEQ ID NOS:861 and 864, SEQ ID NOS:874 and 825, SEQ ID NOS:875 and 879, SEQ ID NOS:876 and 880, SEQ ID NOS:877 and 881, or SEQ ID NOS:878 and 882.

### Nucleic Acids, Vectors, Host Cells, and Methods for Making Heterodimers

In a related aspect, the present disclosure also provides isolated nucleic acid (used interchangeably with “polynucleotide”) molecules that encode single chain polypeptides provided herein. Exemplary nucleic acid molecules (either with or without a nucleotide sequence encoding a signal peptide sequence) are set forth in SEQ ID NOS:1, 3, 5, 7, 9, 11, 13, 15, 17, 19, 21, 23, 25-28, 33-52 and 792-808.

The present disclosure also provides vectors that comprise nucleic acid sequence encoding single chain polypeptides provided herein. As used herein, “vector” refers to a nucleic acid molecule capable of transporting another nucleic acid to which it has been linked. Exemplary vectors include plasmids, yeast artificial chromosomes, and viral genomes. Certain vectors can autonomously replicate in a host cell, while other vectors can be integrated into the genome of a host cell and thereby are replicated with the host genome.

In certain embodiments, the vectors may be recombinant expression vectors. “Recombinant expression vectors” or “expression vectors” refer to vectors that contain nucleic acid sequences that are operatively linked to an expression control sequence (e.g., a promoter) and are thus capable of directing the expression of those sequences.

Promoter sequences useful in expression vectors provided herein can be selected from any desired gene using CAT (chloramphenicol transferase) vectors or other vectors with selectable markers. Eukaryotic promoters include CMV immediate early, HSV thymidine kinase, early and late SV40, LTRs from retrovirus, and mouse metallothionein-I. In certain embodiments, the promoters are inducible promoters.

In certain embodiments, a vector is an expression vector that comprises a nucleic acid sequence encoding a first single chain polypeptide of a polypeptide heterodimer provided herein. In certain other embodiments, a vector is an expression vector that comprises a nucleic acid sequence encoding a second single chain polypeptide of a polypeptide heterodimer provided herein.

In certain embodiments, a vector is an expression vector that comprises nucleic acid sequences encoding both first and second single chain polypeptides of a polypeptide heterodimer. The promoter for the nucleic acid sequence encoding the first single chain polypeptide may be the same as the promoter for the nucleic acid encoding the second single chain polypeptide. Alternatively, the promoter for the nucleic acid sequence encoding the first single chain polypeptide may be different from the promoter for the nucleic acid encoding the second single chain polypeptide so that the expression level of the first and second single chain polypeptides may be differentially modulated to maximum heterodimerization of the first and second single chain polypeptides. In certain embodiments, one or both the promoters for the nucleic acid encoding the first and second single chain polypeptides are inducible promoters.

The present disclosure also provides a host cell transformed or transfected with, or otherwise containing, any of the nucleic acids or vectors provided herein. Exemplary host cells include VERO cells, HeLa cells, Chinese hamster ovary (CHO) cell lines (including modified CHO cells capable of modifying the glycosylation pattern of expressed multivalent binding molecules, see US Patent Application Publication No. 2003/0115614), COS cells (such as COS-7), W138, BHK, HepG2, 3T3, RIN, MDCK, A549, PC12, K562, HEK293 cells, HepG2 cells, N cells, 3T3 cells, Spodoptera frugiperda cells (e.g., Sf9 cells), Saccharomyces cerevisiae cells, Escherichia coli, Bacillus subtilis, Salmonella typhimurium, or members of the Streptomycete family.

In certain embodiments, a host cell comprises a first expression vector containing a nucleic acid encoding a first single chain polypeptide and a second expression vector containing a nucleic acid encoding a second single chain polypeptide.

In certain other embodiments, a host cell comprises an expression vector containing a nucleic acid encoding both first and second single chain polypeptides.

The disclosure also includes a method of producing polypeptide heterodimers described herein. In certain embodiments, the method comprises culturing a host cell that comprises nucleic acids encoding both the first and second single chain polypeptides under conditions suitable to express the polypeptides, and optionally isolating or purifying the heterodimers formed from the first and second single chain polypeptides from the culture. The nucleic acid encoding the first single chain polypeptide and the nucleic acid encoding the second single chain polypeptide may be present in a single expression vector in the host cell or in two different expression vectors in the host cells. In the latter case, the ratio between the two expression vectors may be controlled to maximize heterodimerization of the first and second single chain polypeptides.

The present disclosure provides purified polypeptide heterodimers as described herein. The term “purified,” as used herein, refers to a composition, isolatable from other components, wherein the polypeptide heterodimer is enriched to any degree relative to its naturally obtainable state. In certain embodiments, the present disclosure provides substantially purified polypeptide heterodimers as described herein. “Substantially purified” refers to a polypeptide heterodimer composition in which the polypeptide heterodimer forms the major component of the composition, such as constituting at least about 50%, such as at least about 60%, about 70%, about 80%, about 90%, about 95%, about 99%, of the polypeptides, by weight, in the composition.

Protein purification techniques are well known to those of skill in the art. These techniques involve, at one level, the crude fractionation of the polypeptide and non-polypeptide fractions. Further purification using chromatographic and electrophoretic techniques to achieve partial or complete purification (or purification to homogeneity) is frequently desired. Analytical methods particularly suited to the preparation of a pure fusion protein are ion-exchange chromatography, size exclusion chromatography; polyacrylamide gel electrophoresis; and isoelectric focusing. Particularly efficient methods of purifying peptides are fast protein liquid chromatography and HPLC.

Various methods for quantifying the degree of purification are known to those of skill in the art in light of the present disclosure. These include, for example, assessing the amount of polypeptide heterodimers in a fraction by SDS/PAGE analysis and HPLC as illustrated in the examples provided herein.

The method for making polypeptide heterodimers provided herein is advantageous over a method for first expressing and purifying separately individual single chain polypeptides and then incubating purified individual single chain polypeptides together to form polypeptide heterodimers. For example, certain single chain polypeptides (e.g., certain polypeptides containing only CH1 regions as their immunoglobulin heterodimerization domains) are unstable when expressed alone. In addition, separate expression and purification of individual single chain polypeptides followed by combining the purified individual single chain polypeptides involve more steps than coexpressing both single chain polypeptides followed by purifying resulting polypeptide heterodimers and generally less efficient.

### Compositions and Methods for Using Heterodimers

In addition to polypeptide heterodimers, the present disclosure also provides pharmaceutical compositions and unit dose forms that comprise the polypeptide heterodimers as well as methods for using the polypeptide heterodimers, the pharmaceutical compositions and unit dose forms.

Compositions of polypeptide heterodimers of this disclosure generally comprise a polypeptide heterodimer provided herein in combination with a pharmaceutically acceptable excipient, including pharmaceutically acceptable carriers and diluents. Pharmaceutical acceptable excipients will be nontoxic to recipients at the dosages and concentrations employed. They are well known in the pharmaceutical art and described, for example, in Rowe et al., Handbook of Pharmaecutical Excipients: A Comprehensive Guide to Uses, Properties, and Safety, 5^{th} Ed., 2006.

Pharmaceutically acceptable carriers for therapeutic use are also well known in the pharmaceutical art, and are described, for example, in Remington's Pharmaceutical Sciences, Mack Publishing Co. (A. R. Gennaro (Ed.) 1985). Exemplary pharmaceutically acceptable carriers include sterile saline and phosphate buffered saline at physiological pH. Preservatives, stabilizers, dyes and the like may be provided in the pharmaceutical composition. In addition, antioxidants and suspending agents may also be used.

Pharmaceutical compositions may also contain diluents such as buffers, antioxidants such as ascorbic acid, low molecular weight (less than about 10 residues) polypeptides, proteins, amino acids, carbohydrates (e.g., glucose, sucrose, dextrins), chelating agents (e.g., EDTA), glutathione and other stabilizers and excipients. Neutral buffered saline or saline mixed with nonspecific serum albumin are exemplary diluents. For instance, the product may be formulated as a lyophilizate using appropriate excipient solutions (e.g., sucrose) as diluents.

The present disclosure also provides a method for treating a disease or disorder associated with, for example, excessive receptor-mediated signal transduction, comprising administering to a patient in need thereof an effective amount of a polypeptide heterodimer comprising a binding domain that specifically binds a receptor, such as a receptor tyrosine kinase, including EGFR, EGFRvIII, ErbB2, ErbB3, ErbB4, IGF1R, c-Met, RON, and EphA2.

Exemplary diseases or disorders associated with excess receptor-mediated signal transduction include cancer (e.g., solid malignancy and hematologic malignancy), autoimmune or inflammatory diseases or conditions, sepsis resulting from bacterial infection, and viral infection.

In one aspect, the present disclosure provides a method for directing T cell activation, comprising administering to a patient in need thereof an effective amount of a polypeptide heterodimer that comprises a binding domain that specifically binds TCRα, TCRβ, CD3γ, CD3δ, CD3ε or a combination thereof, and a second binding domain that specifically binds a different target, for instance, a tumor-specific antigen or other antigen of choice at a site or cell where T cell activation is desired.

In another aspect, the present disclosure provides a method for inhibiting growth, metastasis or metastatic growth of a malignancy (e.g., a solid malignancy or a hematologic malignancy), comprising administering to a patient in need thereof an effective amount of a polypeptide heterodimer provided herein or a composition thereof.

A wide variety of cancers, including solid malignancy and hematologic malignancy, are amenable to the compositions and methods disclosed herein. Types of cancer that may be treated include, but are not limited to: adenocarcinoma of the breast, prostate, pancreas, colon and rectum; all forms of bronchogenic carcinoma of the lung (including squamous cell carcinoma, adenocarcinoma, small cell lung cancer and non-small cell lung cancer); myeloid; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, merkel cell, mucinous, non-small cell lung, oat cell, papillary, scirrhous, bronchiolar, bronchogenic, squamous cell, and transitional cell). Additional types of cancers that may be treated include: histiocytic disorders; leukemia; histiocytosis malignant; Hodgkin's disease; immunoproliferative small; non-Hodgkin's lymphoma; plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; osteoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioneuroma; glioma; medulloblastoma; meningioma; neurilemmoma; neuroblastoma; neuroepithelioma; neurofibroma; neuroma; paraganglioma; paraganglioma nonchromaffin; and glioblastoma multiforme. The types of cancers that may be treated also include, but are not limited to, angiokeratoma; angiolymphoid hyperplasia with eosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pinealoma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; and cervical dysplasia.

Additional exemplary cancers that are also amenable to the compositions and methods disclosed herein are B-cell cancers, including B-cell lymphomas [such as various forms of Hodgkin's disease, non-Hodgkins lymphoma (NHL) or central nervous system lymphomas], leukemias [such as acute lymphoblastic leukemia (ALL), chronic lymphocytic leukemia (CLL), Hairy cell leukemia and chronic myoblastic leukemia] and myelomas (such as multiple myeloma). Additional B cell cancers include small lymphocytic lymphoma, B-cell prolymphocytic leukemia, lymphoplasmacytic lymphoma, splenic marginal zone lymphoma, plasma cell myeloma, solitary plasmacytoma of bone, extraosseous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt lymphoma/leukemia, B-cell proliferations of uncertain malignant potential, lymphomatoid granulomatosis, and post-transplant lymphoproliferative disorder.

In certain embodiments, polypeptide heterodimers useful for inhibiting growth of a solid malignancy or metastasis or metastatic growth of a solid malignancy or a hematologic malignancy include those that specifically bind to a tumor or cancer antigen and a T cell target. Such heterodimers are typically designed to have a higher binding affinity to the tumor or cancer antigen (e.g., at least about 5, 10, 15, 20, 25, 50, 75 or 100 folds higher) than to the T cell target so that they preferentially bind to the tumor or cancer antigen first and subsequently bind to the T cell target, which in turn recruit and activate T cells to damage or destroy the tumor or cancer cells carrying the tumor or cancer antigen on their surface. Exemplary tumor or cancer antigens and T cell targets include those provided above in the section describing binding domains of polypeptide heterodimers. For instance, a T cell target can be CD3 or PSMA.

In certain embodiments, polypeptide heterodimers useful for inhibiting growth, metastasis, or metastatic growth of a malignancy, or by directed T cell activation, comprise at least one binding domain that specifically binds an oncology target (including a tumor or cell antigen, such as RON, c-Met, CEACAM-6, or PSMA) and another binding domain that specifically binds a TCR complex or a component thereof (e.g., TCRα, TCRβ, CD3γ, CD3δ, and CD3ε).

In certain other embodiments, polypeptide heterodimers useful for inhibiting growth, metastasis, or metastatic growth of a malignancy comprise at least one binding domain that specifically binds CD28 and another binding domain that specifically binds CD79b. Such polypeptide heterodimers may further comprise a binding domain that comprises a PDL2 ectodomain, a binding domain that specifically binds hyperIL-6, or both binding domains.

In certain other embodiments, polypeptide heterodimers useful for inhibiting growth, metastasis, or metastatic growth of a malignancy comprise at least one binding domain that specifically binds RON and another binding domain that specifically binds c-Met.

In certain other embodiments, polypeptide heterodimers useful for inhibiting growth, metastasis, or metastatic growth of a malignancy comprise binding domains that specifically bind one or more of the following receptor tyrosine kinases: c-Met, RON, EGFR, EGFRvIII, Her2, ErbB3, ErbB4, and IGF1R, EphA2. In certain embodiments, the heterodimers may further comprise one or more binding domains that specifically bind to one or more of the following antiangiogenic agents: PDGFR, VEGFR1-4, and angiopoietin 2. In certain embodiments, the above heterodimers may further comprise one or more binding domains that specifically bind to one or more the following Fc receptors to increase targeting of cytotoxic effector function: CD64, CD32A and CD16. In certain embodiments, the above heterodimers may further comprise a binding domain that specifically binds the transferrin receptor (CD71) to enable degradation of the receptors.

In certain other embodiments, polypeptide heterodimers useful for inhibiting growth, metastasis, or metastatic growth of a malignancy comprise binding domains that are agonists of two or more of the following TNFSFRs: TNFR1, TNFR2, DR4, DR5, TWEAKR, and FAS.

In certain other embodiments, polypeptide heterodimers useful for inhibiting growth, metastasis, or metastatic growth of a malignancy comprise binding domains that specifically bind two or more of the following pro-oncogenic cytokines or growth factors: HGF, MSP, EGF (including epiregulin, herregulin, β-regulin, neuregulin), HIF-1α, VEGFA, VEGFB, VEGFC, VEGFD, TNFα, IL-6, hyperIL-6, IL-8, Wnt, sHH, TGFβ, or PDGF.

In certain embodiments, polypeptide heterodimers useful for inhibiting growth of a solid malignancy or metastasis or metastatic growth of a solid malignancy include those that specifically bind to, for example, EGFR, ErbB3, ErbB4, c-Met, RON, EphA2, IGF1R, VEGFR1, VEGFR2, VEGFR3, CD44v6, CD151, EpCAM, CEACAM6, TGFBR2, GHRHR, GHR, IL-6R, gp130, TNFR2, PD1, TWEAK-R, OSMRβ, Patched-1, Frizzled, or Robo1.

Polypeptide heterodimers useful for inhibiting metastasis or metastatic growth of a hematologic malignancy include those that specifically bind to, for example, EGFR, ErbB3, c-Met, RON, EphA2, IGF1R, TGFBR2, IL-6R, gp130, TNFR2, PD1, OSMRβ, LTβR, CD19, CD80, CD81, or CD86.

In another aspect, the present disclosure provides a method for treating an autoimmune or inflammatory disease, disorder or condition, comprising administering to a patient in need thereof an effective amount of a polypeptide heterodimer provided herein or a composition thereof.

Exemplary autoimmune or inflammatory diseases, disorders or conditions that may be treated by the fusion proteins and compositions and unit dose forms thereof include, and are not limited to, inflammatory bowel disease (e.g., Crohn's disease or ulcerative colitis), diabetes mellitus (e.g., type I diabetes), dermatomyositis, polymyositis, pernicious anaemia, primary biliary cirrhosis, acute disseminated encephalomyelitis (ADEM), Addison's disease, ankylosing spondylitis, antiphospholipid antibody syndrome (APS), autoimmune hepatitis, Goodpasture's syndrome, Graves' disease, Guillain-Barré syndrome (GBS), Hashimoto's disease, idiopathic thrombocytopenic purpura, systemic lupus erythematosus, lupus nephritis, neuropsychiatric lupus, multiple sclerosis (MS), myasthenia gravis, pemphigus vulgaris, asthma, psoriatic arthritis, rheumatoid arthritis, Sjögren's syndrome, temporal arteritis (also known as “giant cell arteritis”), autoimmune hemolytic anemia, Bullous pemphigoid, vasculitis, coeliac disease, chronic obstructive pulmonary disease, endometriosis, Hidradenitis suppurativa, interstitial cystitis, morphea, scleroderma, narcolepsy, neuromyotonia, vitiligo, and autoimmune inner ear disease.

Exemplary polypeptide heterodimers useful for treating an autoimmune or inflammatory disease, disorder or condition may comprises one or more binding domain that specifically binds CD28, and one, two, or three of the following additional binding domains: a binding domain that comprises PDL2 ectodomain, a binding domain that comprises monoIL-10, and a binding domain that specifically binds CD86. For example, a polypeptide heterodimer may comprise a binding domain that specifically binds CD28 and one to three binding domains that specifically bind CD86.

Additional exemplary polypeptide heterodimers useful for treating an autoimmune or inflammatory disease, disorder or condition may comprise one or more binding domains that specifically bind CD28 and one or more binding domains that are an IL-10 agonist (e.g., monoIL-10), an HLA-G agonist, an HGF agonist, an IL-35 agonist, a PD-1 agonist, a BTLA agonist, a LIGHT antagonist, a GITRL antagonist or a CD40 antagonist. Alternatively, polypeptide heterodimers may comprise two or more binding domains selected from an IL-10 agonist (e.g., monoIL-10), an HLA-G agonist, an HGF agonist, an IL-35 agonist, a PD-1 agonist, a BTLA agonist, a LIGHT antagonist, a GITRL antagonist or a CD40 antagonist.

Additional exemplary polypeptide heterodimers useful for treating an autoimmune or inflammatory disease, disorder or condition may comprise one or more binding domains that specifically bind CD32B and one or more binding domains that are an IL-10 agonist (e.g., monoIL-10), an HLA-G agonist, an HGF agonist, an IL-35 agonist, a PD-1 agonist, a BTLA agonist, a LIGHT antagonist, a GITRL antagonist or a CD40 antagonist.

Additional exemplary polypeptide heterodimers useful for treating an autoimmune or inflammatory disease, disorder or condition may comprise binding domains that specifically bind one or more of the following TNFSFRs: TNFR1, TNFR2, HVEW, LTβR, TWEAKR, TACI, BAFF-R, BCMA, or FAS.

Additional exemplary polypeptide heterodimers useful for treating an autoimmune or inflammatory disease, disorder or condition may comprise binding domains that specifically bind two or more of the following pro-inflammatory cytokines/chemokines, such as TNFα, IL-6, IL-2, IL-1, IL-8, IP-10, IFNγ, IFNα, RANKL, FASL, TGFβ, IL7, IL10, IL17A/F, TWEAK, CSF2, IGF1, IGF2 or BLyS/APRIL.

Polypeptide heterodimers useful for treating an autoimmune or inflammatory disease, disorder or condition include those that specifically bind to, for example, TGFBR2, IL-6R, gp130, TNFR1, TNFR2, PD1, HVEM, OX40, CD40, CD137, TWEAK-R, LTβR, LIFRβ, OSMRβ, CD3, TCRα, TCRβ, CD19, CD28, CD80, CD81, CD86, TLR7, TLR9, or any combination thereof.

In another aspect, the present disclosure provides a method for treating a B-cell associated disorder or disease that comprises administering to a patient in need thereof (e.g., a patient having or suspected of having a B-cell associated disorder or disease) an effective amount of a polypeptide heterodimer provided herein, such as those that specifically bind a B cell target.

Polypeptide heterodimers useful in treating a B-cell associated disorder or disease may comprise binding domains that specifically bind one or more B-cell targets, such as CD79b, CD19, HLA-DR, CD20, CD21, CD22, CD30, CD33, CD37, CD38, or CD70. The polypeptide heterodimers may further comprise a binding domain that specifically binds a T-cell target, such as a TCR complex or a component thereof, including TCRα, TCRβ, CD3γ, CD3δ, or CD3ε.

Polypeptide heterodimers useful in treating a B-cell associated disorder or disease may comprise a binding domain that specifically bind a B-cell target, such as CD79b, CD19, HLA-DR, CD20, CD21, CD22, CD30, CD33, CD37, CD38, and CD70, and a binding domain that specifically binds CD64, CD32A, or CD16 to increase targeting of cytotoxic effector function.

The polypeptide heterodimers or compositions thereof of the present disclosure may be administered orally, topically, transdermally, parenterally, by inhalation spray, vaginally, rectally, or by intracranial injection, or any combination thereof. In one embodiment, the polypeptide heterodimers or compositions thereof are administered parenterally. The term “parenteral,” as used herein, includes subcutaneous injections, intravenous, intramuscular, intracisternal injection, or infusion techniques. Administration by intravenous, intradermal, intramusclar, intramammary, intraperitoneal, intrathecal, retrobulbar, intrapulmonary injection and/or surgical implantation at a particular site is contemplated as well. For instance, the invention includes administering polypeptide heterodimers or compositions thereof by intravenous injection.

The pharmaceutically effective dose depends on the type of disease, the composition used, the route of administration, the type of subject being treated, the physical characteristics of the specific subject under consideration for treatment, concurrent medication, and other factors that those skilled in the medical arts will recognize. For example, an amount between 0.01 mg/kg and 1000 mg/kg (e.g., about 0.1 to 1 mg/kg, about 1 to 10 mg/kg, about 10-50 mg/kg, about 50-100 mg/kg, about 100-500 mg/kg, or about 500-1000 mg/kg) body weight (which can be administered as a single dose, daily, weekly, monthly, or at any appropriate interval) of active ingredient may be administered depending on the potency of a polypeptide heterodimer of this disclosure.

Also contemplated is the administration of polypeptide heterodimers or compositions thereof in combination with a second agent. A second agent may be one accepted in the art as a standard treatment for a particular disease state or disorder, such as in cancer, inflammation, autoimmunity, and infection. Exemplary second agents contemplated include polypeptide heterodimers that bind to targets different from those that primary polypeptide heterodimers bind, polyclonal antibodies, monoclonal antibodies, immunoglobulin-derived fusion proteins, chemotherapeutics, ionizing radiation, steroids, NSAIDs, anti-infective agents, or other active and ancillary agents, or any combination thereof.

In certain embodiments, a polypeptide heterodimer and a second agent act synergistically. In other words, these two compounds interact such that the combined effect of the compounds is greater than the sum of the individual effects of each compound when administered alone (see, e.g., Berenbaum, Pharmacol. Rev. 41:93, 1989).

In certain other embodiments, a polypeptide heterodimer and a second agent act additively. In other words, these two compounds interact such that the combined effect of the compounds is the same as the sum of the individual effects of each compound when administered alone.

Second agents useful in combination with polypeptide heterodimers or compositions thereof provided herein may be steroids, NSAIDs, mTOR inhibitors (e.g., rapamycin (sirolimus), temsirolimus, deforolimus, everolimus, zotarolimus, curcumin, farnesylthiosalicylic acid), calcineurin inhibitors (e.g., cyclosporine, tacrolimus), anti-metabolites (e.g., mycophenolic acid, mycophenolate mofetil), polyclonal antibodies (e.g., anti-thymocyte globulin), monoclonal antibodies (e.g., daclizumab, basiliximab), and CTLA4-Ig fusion proteins (e.g., abatacept or belatacept).

Second agents useful for inhibiting growth of a solid malignancy, inhibiting metastasis or metastatic growth of a solid malignancy, or treating or ameliorating a hematologic malignancy include chemotherapeutic agents, ionizing radiation, and other anti-cancer drugs. Examples of chemotherapeutic agents contemplated as further therapeutic agents include alkylating agents, such as nitrogen mustards (e.g., mechlorethamine, cyclophosphamide, ifosfamide, melphalan, and chlorambucil); bifunctional chemotherapeutics (e.g., bendamustine); nitrosoureas (e.g., carmustine (BCNU), lomustine (CCNU), and semustine (methyl-CCNU)); ethyleneimines and methyl-melamines (e.g., triethylenemelamine (TEM), triethylene thiophosphoramide (thiotepa), and hexamethylmelamine (HMM, altretamine)); alkyl sulfonates (e.g., buslfan); and triazines (e.g., dacabazine (DTIC)); antimetabolites, such as folic acid analogues (e.g., methotrexate, trimetrexate, and pemetrexed (multi-targeted antifolate)); pyrimidine analogues (such as 5-fluorouracil (5-FU), fluorodeoxyuridine, gemcitabine, cytosine arabinoside (AraC, cytarabine), 5-azacytidine, and 2,2′-difluorodeoxycytidine); and purine analogues (e.g., 6-mercaptopurine, 6-thioguanine, azathioprine, 2′-deoxycoformycin (pentostatin), erythrohydroxynonyladenine (EHNA), fludarabine phosphate, 2-chlorodeoxyadenosine (cladribine, 2-CdA)); Type I topoisomerase inhibitors such as camptothecin (CPT), topotecan, and irinotecan; natural products, such as epipodophylotoxins (e.g., etoposide and teniposide); and vinca alkaloids (e.g., vinblastine, vincristine, and vinorelbine); anti-tumor antibiotics such as actinomycin D, doxorubicin, and bleomycin; radiosensitizers such as 5-bromodeozyuridine, 5-iododeoxyuridine, and bromodeoxycytidine; platinum coordination complexes such as cisplatin, carboplatin, and oxaliplatin; substituted ureas, such as hydroxyurea; and methylhydrazine derivatives such as N-methylhydrazine (MIH) and procarbazine.

In certain embodiments, second agents useful for inhibiting growth metastasis or metastatic growth of a malignancy include polypeptide heterodimers according to the present disclosure that bind to cancer cell targets other than the target that the first polypeptide heterodimer binds. In certain other embodiments, second agents useful for such treatments include polyclonal antibodies, monoclonal antibodies, and immunoglobulin-derived fusion proteins that bind to cancer cell targets. Exemplary cancer cell targets are provided above in the context of describing targets of polypeptide heterodimers useful for the above-noted treatment.

Further therapeutic agents contemplated by this disclosure for treatment of autoimmune diseases are referred to as immunosuppressive agents, which act to suppress or mask the immune system of the individual being treated. Immunosuppressive agents include, for example, non-steroidal anti-inflammatory drugs (NSAIDs), analgesics, glucocorticoids, disease-modifying antirheumatic drugs (DMARDs) for the treatment of arthritis, or biologic response modifiers. Compositions in the DMARD description are also useful in the treatment of many other autoimmune diseases aside from rheumatoid arthritis.

Exemplary NSAIDs are chosen from the group consisting of ibuprofen, naproxen, naproxen sodium, Cox-2 inhibitors such as Vioxx and Celebrex, and sialylates. Exemplary analgesics are chosen from the group consisting of acetaminophen, oxycodone, tramadol of proporxyphene hydrochloride. Exemplary glucocorticoids are chosen from the group consisting of cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists (e.g. etanercept (Enbrel), adalimumab (Humira) and infliximab (Remicade)), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular) and minocycline.

Additional second agents useful for treating an autoimmune or inflammatory disease, disorder or condition may be a polyclonal or monoclonal antibody, an immunoglobulin-derived fusion protein (e.g., scFv, SMIP™, PIMS, SCORPION™ fusion proteins), or a polypeptide heterodimer according to the present disclosure that specifically bind a target associated with such a disease, disorder or condition. Examples of such targets are provided above in the context of targets of polypeptide heterodimers of the present disclosure useful in the above-noted treatment.

It is contemplated the binding molecule composition and the second active agent may be given simultaneously in the same formulation. Alternatively, the second agents may be administered in a separate formulation but concurrently (i.e., given within less than one hour of each other).

In certain embodiments, the second active agent may be administered prior to administration of a polypeptide heterodimer or a composition thereof. Prior administration refers to administration of the second active agent at least one hour prior to treatment with the polypeptide heterodimer or the composition thereof. It is further contemplated that the active agent may be administered subsequent to administration of the binding molecule composition. Subsequent administration is meant to describe administration at least one hour after the administration of the polypeptide heterodimer or the composition thereof.

This disclosure contemplates a dosage unit comprising a pharmaceutical composition of this disclosure. Such dosage units include, for example, a single-dose or a multi-dose vial or syringe, including a two-compartment vial or syringe, one comprising the pharmaceutical composition of this disclosure in lyophilized form and the other a diluent for reconstitution. A multi-dose dosage unit can also be, e.g., a bag or tube for connection to an intravenous infusion device.

This disclosure also contemplates a kit comprising a pharmaceutical composition of this disclosure in unit dose, or multi-dose, container, e.g., a vial, and a set of instructions for administering the composition to patients suffering a disorder such as a disorder described above.

### EXAMPLES

### Example 1

### Bivalent Polypeptide Heterodimer with Two Pairs of Heterodimerization Domains

Polypeptide heterodimer X0172 was made by co-expressing single chain polypeptides X0130 (2E12 CH1 CH2 CH3 Ck) and X0168 (Ck CH2 CH3 CH1 H68 2E12). Single chain polypeptide X0130 comprises, from its amino to carboxyl terminus: 2E12 (anti-CD28) scFv, human IgG1 CH1, human IgG1 SCC-P hinge, human IgG1 CH2, human IgG1 CH3, and altered human Ck (without the first Arg or the last Cys). The nucleic acid and amino acid sequences of X130 are set forth in SEQ ID NOS:1 and 2, respectively. Single chain polypeptide X0168 comprises, from its amino to carboxyl terminus: altered human Ck (without the first Arg or the last Cys), human IgG1 SCC-P hinge, human IgG1 CH2, human IgG1 CH3, human IgG1 CH1, H68 linker, and 2E12 (anti-CD28) scFv. The nucleic acid and amino acid sequences of X0168 are set forth in SEQ ID NOS:3 and 4, respectively. The amino acid sequence of H68 linker is set forth in SEQ ID NO:78.

For comparison, polypeptide heterodimer X0124 was made by co-expressing single chain polypeptide X0112 (2E12 CH1 CH2 CH3) and X0113 (Ck CH2 CH3). Single chain polypeptide X0112 comprises, from its amino to carboxyl terminus: 2E12 (anti-CD28) scFv, human IgG1 CH1, human IgG1 SCC-P hinge, human IgG1 CH2, and human IgG1 CH3. The nucleic acid and amino acid sequences of X0112 are set forth in SEQ ID NOS:5 and 6, respectively. Single chain polypeptide X0113 comprises, from its amino to carboxyl terminus: altered human Ck (without the first Arg or the last Cys), human IgG1 SCC-P hinge, human IgG1 CH2, and human IgG1 CH3. The nucleic acid and amino acid sequence of X0113 are set forth in SEQ ID NOS:7 and 8, respectively.

### Expression

The day before transfection, HEK293 cells were suspended at a cell concentration of 0.5×10⁶ cells/ml in Freestyle 293 expression medium (Gibco). For a large transfection, 250 ml of cells were used, but for a small transfection, 60 ml of cells were used. On the transfection day, 320 ul of 293fectin reagent (Invitrogen) was mixed with 8 ml of media. At the same time, 250 ug of DNA for each of the two chains were also mixed with 8 ml of media and incubated for 5 minutes. After 15 minutes of incubation, the DNA-293fectin mixture was added to the 250 ml of 293 cells and returned to the shaker at 37° C. and shaken at a speed of 120 RPM. For the smaller transfection using 60 ml of cells, a fourth of the DNA, 293fectin and media were used.

### Purification

Protein A affinity chromatography was used to purify the proteins. 2 mL of packed protein A agarose (Repligen) was added to a Biorad column (Econo-column chromatography column, size 2.5×10 cm), washed extensively with PBS (10× column volume) and the supernatants were loaded, washed with PBS again and eluted with 3 column volumes of Pierce IgG elution buffer. Proteins were then dialyzed extensively against PBS. Proteins were then concentrated using Amicon centrifugal filter devices to a final volume of around 0.5 mL.

For second step purification, Protein L affinity chromatography or cation exchange chromatography were used. For Protein L purification, protein A purified Polypeptide heterodimer was passed over a Protein L agarose column that had been pre-equilibrated with PBS, washed with PBS (10× column volume) and then eluted with Pierce IgG elution buffer. Proteins were then dialyzed against PBS extensively and concentrated using Amicon centrifugal filter devices to a final volume of around 0.5 mL.

Samples (200-300 ug) of previously affinity purified (Protein A or Protein L) Polypeptide heterodimer constructs were dialyzed into 20 mM MES, pH 6.0 (Buffer A) and loaded onto a MonoS 5/50 GL cation exchange column (GE Healthcare) at a flow rate of 2 mL/min, using an AKTA Explorer FPLC. The column was allowed to equilibrate for 5 column volumes (CV) and then run in a gradient format to a mixture of 50%:50% buffer A:buffer B (buffer B being 20 mM MES, 1 M NaCl, pH 6.0) over 20 CV. A following mixture of 100% buffer B was run for 5 CV to clean the column, and the system was run for another 5 CV at 100% buffer A to re-equilibrate prior to the next injection. Peaks were collected and analyzed by SDS-PAGE and electrospray mass spectrometry.

### SDS-PAGE Analysis

Proteins purified were analyzed on a 10% SDS-PAGE gel using Invitrogen's X-cell Surelock gel box.

Synergy with Suboptimal Concentration of PMA

Peripheral blood mononuclear cells (PBMC) from in-house donors were isolated from heparinized whole blood via centrifugation over Lymphocyte Separation Media (MP Biomedicals, Aurora, Ohio) and washed two times with RPMI media (Gibco-Invitrogen, Carlsbad, Calif.). CD4+ T-cells were then enriched from the PBMC using negative selection with a MACS CD4+ T-cell Isolation Kit (Miltenyi Biotec, Auburn, Calif.). The enriched (>95%) CD4+ T-cells were then resuspended at a concentration of 1×10⁶ cell/ml in complete RPMI/10% FCS. Test reagents were prepared at 40 ug/ml (yielding a final concentration of 10 μg/ml) in complete RPMI/10% FCS and added in 50 μl/well to flat-bottom 96-well plates (BD Falcon, San Jose, Calif.). PMA (Phorbol 12 myristate 13-acetate; A.G. Scientific, Inc., San Diego, Calif.) in complete RPMI/10% FCS was added in 50 ul/well at 4 ng/ml (final concentration of 1 ng/ml). Then T-cells in complete RPMI/10% FCS were added at a concentration of 5×10⁴ cells/well in a 50 μl volume, and finally an appropriate amount of complete RPMI/10% FCS was added to each well (typically 50 ul) to bring the final volume to 200 ul/well. The cells were treated with the test samples +/−PMA and incubated for 72 hours at 37° C. in 5% CO₂. One microliter of tritiated thymidine (Amersham Biosciences, Pisctaway, N.J.) in a 1:50 dilution of complete RPMI/10% FCS (50 ul/well) was added to the wells for the last 6 hours of culture. Plates were harvested onto a Unifilter-96, GF/C microplate (Perkin Elmer, Boston, Mass.) with a Packard Filtermate Harvester (Perkin Elmer, Boston, Mass.). Numbers are expressed as cpm and are the mean of replicate samples.

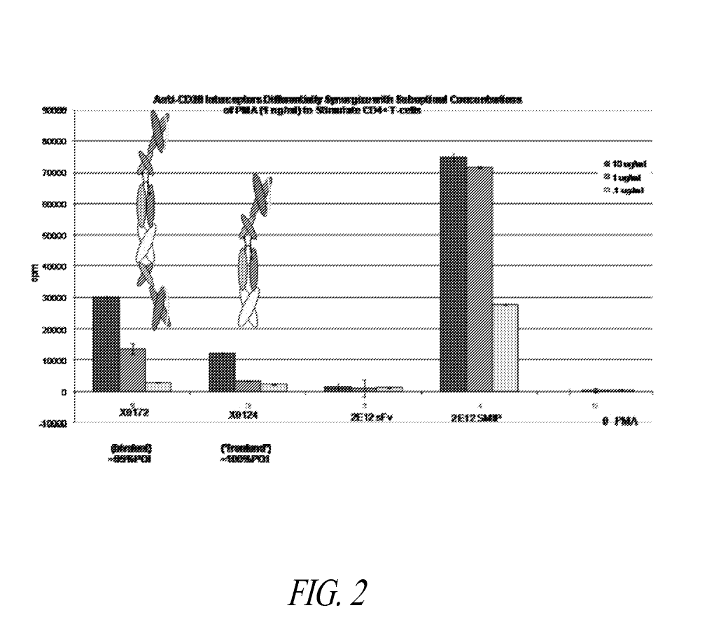

FIG. 2 shows that Polypeptide heterodimer X0172 has a differential property compared to 2E12 SMIP M0039 (SEQ ID NO:77). It did not synergize with PMA as well as the SMIP. This bivalent polypeptide heterodimer has a property closer to the monovalent polypeptide heterodimer, X0124, rather than the bivalent SMIP.

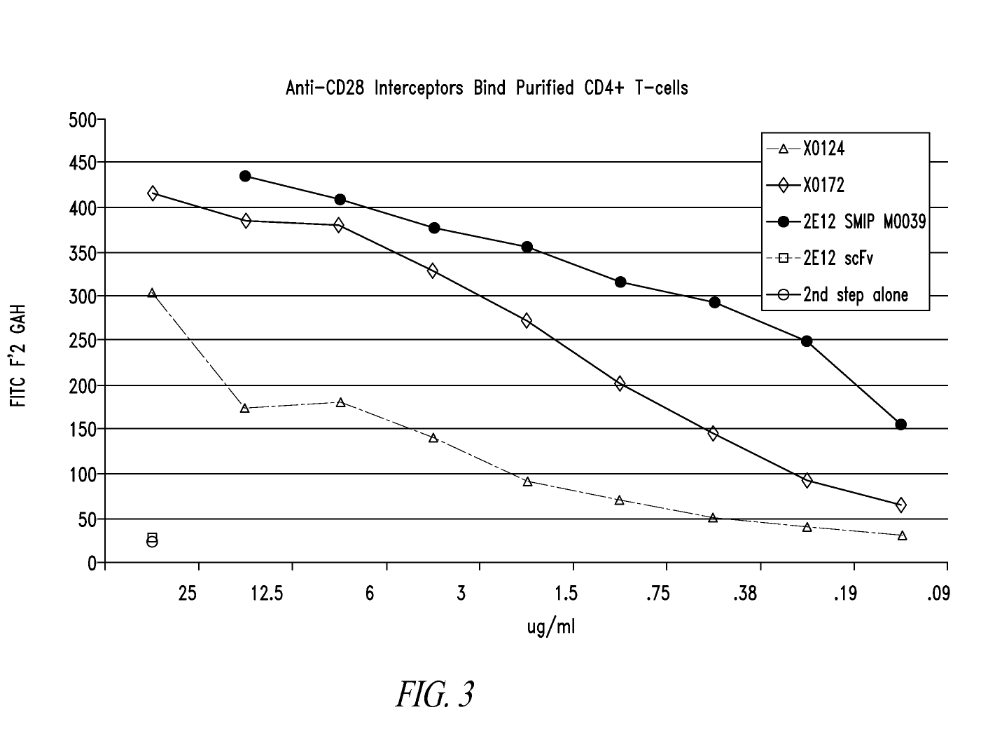

FIG. 3 shows that bivalent polypeptide heterodimer X0172 bound to CD4⁺ cells better than 2E12 scFv (SEQ ID NO:109) and monovalent polypeptide heterodimer X0124.

### Example 2

### Bivalent, Trivalent, Tetravalent Polypeptide Heterodimer Using Single Heterodimerization Domain Pair

Bivalent polypeptide heterodimer X0251 was made by co-expressing single chain polypeptides X0244 (Ck(YAE) CH2(N297A) CH3 H68 2E12) and X0245 (2E12 CH1 CH2(N297A) CH3). Single chain polypeptide X0244 comprises, from its amino to carboxyl terminus: human Ck(YAE) (i.e., human Ck without the first Arg or last Cys but with N30Y, V55A, and T70E substitutions), human IgG1 SCC-P hinge, human IgG1 CH2 (N297A) (i.e., human IgG1 CH2 with a N297A substitution), human IgG1 CH3, H68 linker, and 2E12 (anti-CD28) scFv. The nucleotide and amino acid sequences of X0244 are set forth in SEQ ID NOS:9 and 10, respectively. Single chain polypeptide X0245 comprises, from its amino to carboxyl terminus: 2E12 (anti-CD28) scFv, human IgG1 CH1, human IgG1 SCC-P hinge, human IgG1 CH2 (N297A), and human IgG1 CH3. The nucleotide and amino acid sequences of X0245 are set forth in SEQ ID NOS:11 and 12, respectively.

Trivalent, bispecific polypeptide heterodimer X0252 was made by co-expressing single chain polypeptides X0246 (P2C2 Ck(YAE) CH2(N297A) CH3) and X0247 (2E12 CH1 CH2(N297A) CH3 H68 2E12). Single chain polypeptide X0246 comprises, from its amino to carboxyl terminus: P2C2 (anti-CD79b) scFv, human Ck (YAE), human IgG1 SCC-P hinge, human IgG1 CH2 (N297A), and human IgG1 CH3. The nucleotide and amino acid sequences of X0246 are set forth in SEQ ID NOS:13 and 14, respectively. Single chain polypeptide X0247 comprises, from its amino to carboxyl terminus: 2E12 (anti-CD28) scFv, human IgG1 CH1, human IgG1 SCC-P hinge, human IgG1 CH2 (N297A), human IgG1 CH3, H68 linker, and 2E12 (anti-CD28) scFv. The nucleotide and amino acid sequences of X0247 are set forth in SEQ ID NOS:15 and 16, respectively.

Tetravalent, trispecific polypeptide heterodimer X0253 was made by co-expressing single chain polypeptides X0248 (P2C2 CK(YAE) CH2(N297A) CH3 H68 A2) and X0249 (PDL2 ECD CH1 CH2(N297A) CH3 H68 2E12). Single chain polypeptide X0248 comprises, from its amino to carboxyl terminus: P2C2 (anti-CD79b) scFv, human Ck (YAE), human IgG1 SCC-P hinge, human IgG1 CH2 (N297A), human IgG1 CH3, H68 linker, and A2 (anti-hyperIL-6) scFv. The nucleotide and amino acid sequences of X0248 are set forth in SEQ ID NOS:17 and 18, respectively. Single chain polypeptide X0249 comprises, from its amino to carboxyl terminus: PDL2 ECD (i.e., PDL2 ectodomain), human IgG1 CH1, human IgG1 SCC-P hinge, human IgG1 CH2 (N297A), human IgG1 CH3, H68 linker, and 2E12 (anti-CD28) scFv. The nucleotide and amino acid sequences of X0249 are set forth in SEQ ID NOS:19 and 20, respectively.

Another tetravalent, tetraspecific polypeptide heterodimer X0283, was made by co-expressing single chain polypeptides X0249 (PDL2 ECD CH1 CH2(N297A) CH3 H68 2E12) and X0281 (monoIL-10 Ck(YAE) CH2(N297A) CH3 H68 3D1). Single chain polypeptide X0281 comprises, from its amino to carboxyl terminus: monoIL-10, human Ck(YAE), human IgG1 SCC-P hinge, human IgG1 CH2 (N297A), human IgG1 CH3, H68 linker, and 3D1(anti-CD86) scFv. The nucleotide and amino acid sequences of X0281 are set forth in SEQ ID NOS:21 and 22.

As a control for polypeptide heterodimer X0283, tetravalent heterodimer, polypeptide heterodimer X0284, was made by co-expressing single chain polypeptides X0249 (PDL2 ECD CH1 CH2(N297A) CH3 H68 2E12) and X0282 (monoIL-10 Ck CH2(N297A) CH3 H68 3D1). Single chain polypeptide X0282 is identical to X0281 except that it does not contain N30Y V55A T70E substitutions in the human Ck sequence. Thus, single chain polypeptide X0282 comprises, from its amino to carboxyl terminus: monoIL-10, altered human Ck (without the first Arg or the last Cys), human IgG1 SCC-P hinge, human IgG1 CH2 (N297A), human IgG1 CH3, H68 linker, and 3D1(anti-CD86) scFv. The nucleotide and amino acid sequences of X0282 are set forth in SEQ ID NOS:23 and 24, respectively.

The polypeptide heterodimer molecules were expressed and purified according to Example 1. Purified proteins were analyzed using SDS-PAGE electrophoresis under reduced and non-reduced conditions. Size exclusion chromatography was performed on an AKTA Explorer FPLC (Pharmacia Biotech) using a Superdex200 10/300 GL column. Some proteins were analyzed by electrospray mass spectrometry using an Agilent 6120 TOF ES/MS.

Surface plasmon resonance (SPR) measurements were performed on a Biacore T100 SPR using HBS-P+ (GE Healthcare) as a running buffer. Target was directly immobilized onto a CM5 chip using standard amine coupling chemistry (BIACORE® Amine Coupling Kit, GE Healthcare), with final immobilization levels between 800 and 1900 Ru (resonance units). Polypeptide heterodimer X0283 was injected at 25° C. or 37° C. for 150 seconds at a flow rate of 30 μl/min in a series of concentrations from 10 nM to 1 μM. Dissociation was monitored for 1200 seconds, and the surface was regenerated by injecting 50 mM NaOH for 60 seconds. Binding interactions with the surface were stable through at least 60 regeneration cycles. Data were analyzed using BiaEvaluation for the T100 software (version 2.0, GE Healthcare).

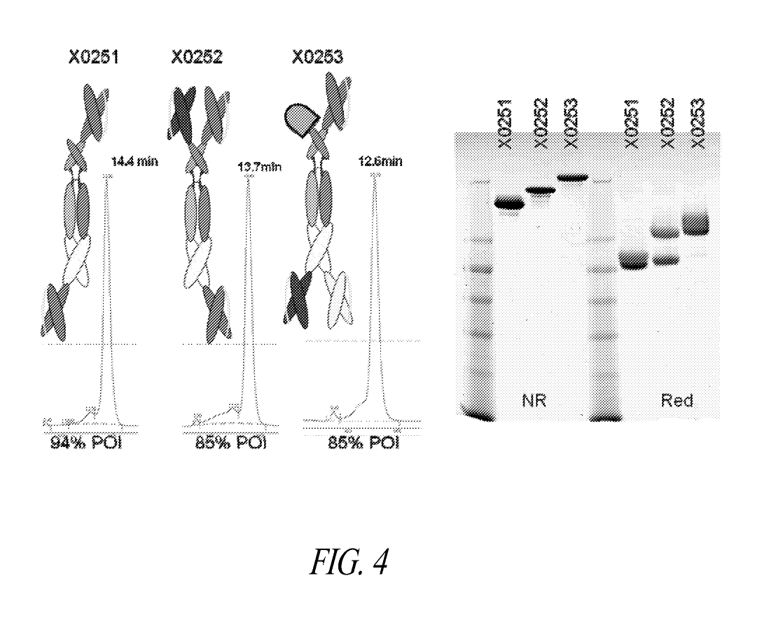

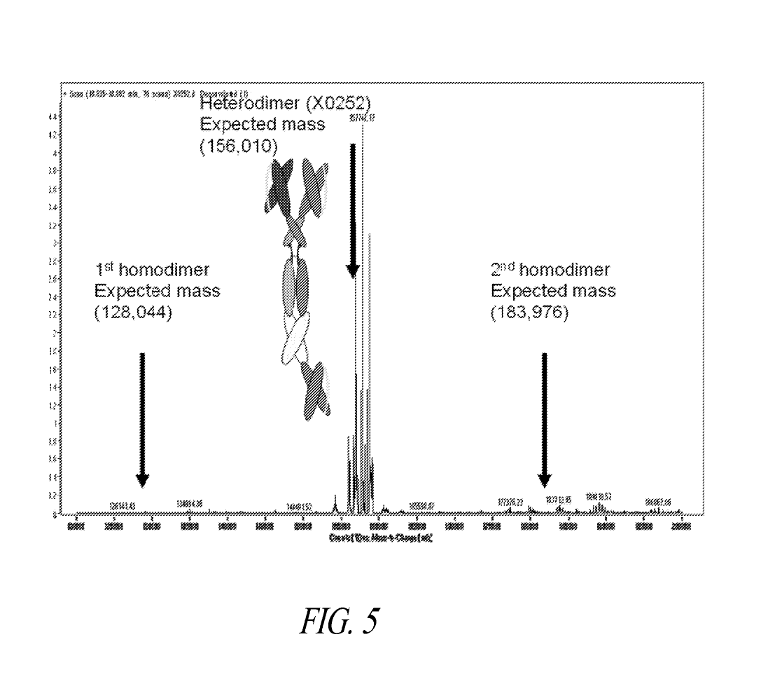

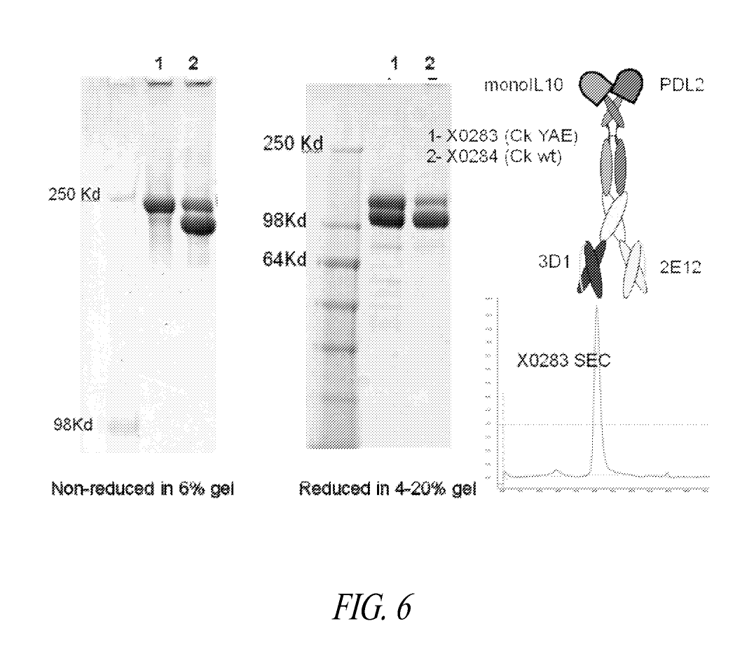

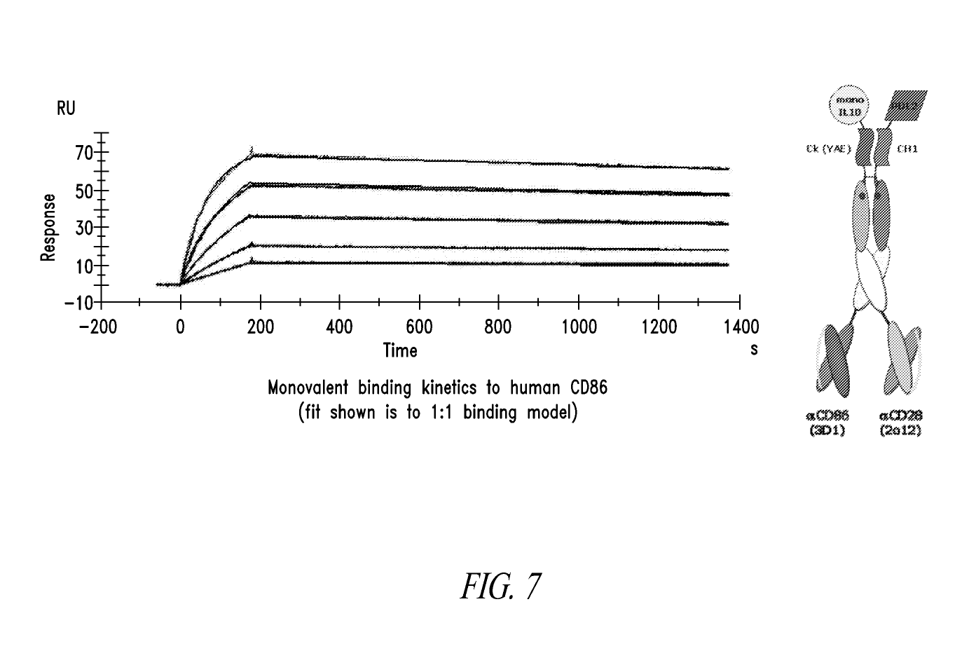

The SDS-PAGE electrophoresis and cation exchange chromatography analyses show that polypeptide heterodimers X0251, X0252 and X0253 were reasonably well expressed and purified (FIG. 4). Mass spectrometry analysis of X0252 shows that the protein is mostly homogeneous with an undetectable amount of the homodimers (FIG. 5). In addition, the SDS-PAGE electrophoresis and cation exchange chromatography analyses of polypeptide heterodimers X0283 and X0284 show that polypeptide heterodimer X0283 having the CH1/Ck(YAE) heterodimerization domains efficiently assembled into a heterodimer compared to control polypeptide heterodimer X0284 having the CH1/Ck heterodimerization domains, which formed homodimers as well (FIG. 6). The Biocore analysis further shows that the 3D1 (anti-CD86) binding domain at the carboxyl terminus of single chain polypeptide X0281 monovalently bound to CD86 (FIG. 7).

### Example 3

### Polypeptide Heterodimers with Anti-RON and Anti-c-Met Binding Domains

A bivalent polypeptide heterodimer with anti-RON binding domains (ORN151) and two bispecific polypeptide heterodimers comprising anti-RON and anti-cMet binding domains (ORN152 and ORN153) were made.

Bivalent polypeptide heterodimer ORN151 comprises single chain polypeptides ORN145 (4C04 CH2 CH3 CH1) and ORN148 (11H09CH2 CH3 Ck(YAE)). Single chain polypeptide ORN145 comprises from its amino to carboxyl terminus: 4C04 (anti-RON) scFv, human IgG1 SCC-P hinge, human IgG1 CH2, human IgG1 CH3 and human IgG1 CH1. The nucleotide and amino acid sequences of ORN145 are set forth in SEQ ID NOS:26 and 30, respectively. Single chain polypeptide ORN148 comprises from its amino to carboxyl terminus: 11H09 (anti-RON) scFv, human IgG1 SCC-P hinge, human CH2, human CH3, and human Ck(YAE). The nucleotide and amino acid sequences of ORN148 are set forth in SEQ ID NOS:28 and 32, respectively.

Bispecific (c-Met, RON) polypeptide heterodimer ORN152 comprises single chain polypeptides ORN116 (MET021 CH2 CH3 CH1) and ORN146 (4C04 CH2 CH3 Ck(YAE)). Single chain polypeptide ORN116 comprises from its amino to carboxyl terminus: MET021 (anti-c-Met) scFv, human IgG1 SCC-P hinge, human IgG1 CH2, human IgG1 CH3 and human IgG1 CH1. The nucleotide and amino acid sequences of ORN116 are set forth in SEQ ID NOS:25 and 29, respectively. Single chain polypeptide ORN146 comprises from its amino to carboxyl terminus: 4C04 (anti-RON) scFv, human IgG1 SCC-P hinge, human CH2, human CH3, and human Ck(YAE). The nucleotide and amino acid sequences of ORN146 are set forth in SEQ ID NOS:27 and 31, respectively.

Bispecific (c-Met, RON) polypeptide heterodimer ORN153 comprises single chain polypeptides ORN116 (MET021 CH2 CH3 CH1) and ORN148 (11H09CH2 CH3 Ck(YAE)).

Polypeptide heterodimers ORN151, ORN152 and ORN153 were expressed according to Example 1. The following expression levels were obtained: 1.9 μg protein/mL of culture for ORN151, 3.1 μg/mL for ORN152, and 4.9 μg/mL for ORN153.

### Example 4

### Polypeptide Heterodimers with Anti-CD3 Binding Domains

Several bispecific polypeptide heterodimers with an anti-CD3 binding domain were made: polypeptide heterodimers S0268, S0269, and TSC020 to TSC030. Polypeptide heterodimer S0268 comprises single chain polypeptides ORN145 (4C04 CH2 CH3 CH1) and TSC019 (G19-4 CH2 CH3 Ck(YAE)). Single chain polypeptide TSC019 comprises from its amino to carboxyl terminus: G19-4 (anti-CD3) scFv, human IgG1 SCC-P hinge, human CH2, human CH3, and human Ck(YAE). The nucleotide and amino acid sequences of TSC019 are set forth in SEQ ID NOS:52 and 72, respectively.

Bispecific (CD3, c-Met) Polypeptide heterodimer S0269 comprises single chain polypeptides ORN160 (5D5 CH2 CH3 CH1) and TSC019 (G19-4 CH2 CH3 Ck(YAE)). Single chain polypeptide ORN160 comprises from its amino to carboxyl terminus: 5D5 (anti-c-Met) scFv, human IgG1 SCC-P hinge, human IgG1 CH2, human IgG1 CH3, and human IgG1 CH1. The nucleotide and amino acid sequences of ORN160 are set forth in SEQ ID NOS:33 and 53, respectively.

Bivalent (CD19) polypeptide heterodimer TSC020 comprises single chain polypeptides TSC001 (HD37 CH2 CH3 CH1) and TSC019 (G19-4 CH2 CH3 Ck(YAE)). Single chain polypeptide TSC001 comprises from its amino to carboxyl terminus: HD37 (anti-CD19) scFv, human IgG1 SCC-P hinge, human IgG1 CH2, human IgG1 CH3 and human IgG1 CH1. The nucleotide and amino acid sequences of TSC001 are set forth in SEQ ID NOS:34 and 54, respectively.

Bispecific (CD20, CD3) polypeptide heterodimer TSC021 comprises single chain polypeptides TSC002 (2H7CH2 CH3 CH1) and TSC019 (G19-4 CH2 CH3 Ck(YAE)). Single chain polypeptide TSC002 comprises from its amino to carboxyl terminus: 2H7 (anti-CD20) scFv, human IgG1 SCC-P hinge, human IgG1 CH2, human IgG1 CH3 and human IgG1 CH1. The nucleotide and amino acid sequences of TSC002 are set forth in SEQ ID NOS:35 and 55, respectively.

Bispecific (CD79b, CD3) polypeptide heterodimer TSC022 comprises single chain polypeptides TSC017 (P2C2H2CH3 CH1) and TSC019 (G19-4 CH2 CH3 Ck(YAE)). Single chain polypeptide TSC017 comprises from its amino to carboxyl terminus: P2C2 (anti-CD79b) scFv, human IgG1 SCC-P hinge, human IgG1 CH2, human IgG1 CH3 and human IgG1 CH1. The nucleotide and amino acid sequences of TSC017 are set forth in SEQ ID NOS:50 and 70, respectively.

Bispecific (HLA-DR, CD3) polypeptide heterodimer TSC023 comprises single chain polypeptides TSC018 (M0042 CH2 CH3 CH1) and TSC019 (G19-4 CH2 CH3 Ck(YAE)). Single chain polypeptide TSC018 comprises from its amino to carboxyl terminus: M0042 (anti-HLA-DR) scFv, human IgG1 SCC-P hinge, human IgG1 CH2, human IgG1 CH3, and human IgG1 CH1. The nucleotide and amino acid sequences of TSC018 are set forth in SEQ ID NOS:51 and 71, respectively.

Bivalent polypeptide heterodimer TSC024 comprises single chain polypeptides TSC010 (HD37-“IgA1 hinge”-CH2 CH3 CH1) and TSC003 (G19-4-“IgA1 hinge”-CH2 CH3 Ck(YAE)). Single chain polypeptide TSC010 comprises from its amino to carboxyl terminus: HD37 (anti-CD19) scFv, altered human IgA1 hinge (PSTPPTPSPSTPPTPSPSCPPCP, SEQ ID NO:752), human IgG1 CH2, human IgG1 CH3, and human IgG1 CH1. The nucleotide and amino acid sequences of TSC010 are set forth in SEQ ID NOS:43 and 63, respectively. Single chain polypeptide TSC003 comprises from its amino to carboxyl terminus: G19-4 (anti-CD3) scFv, altered human IgA1 hinge (SEQ ID NO:752), human IgG1 CH2, human IgG1 CH3, and human Ck(YAE). The nucleotide and amino acid sequences of TSC003 are set forth in SEQ ID NOS:36 and 56, respectively.

Bivalent polypeptide heterodimer TSC025 comprises single chain polypeptides TSC011 (HD37-“IgA2 hinge”-CH2 CH3 CH1) and TSC004 (G19-4-“IgA2 hinge”-CH2 CH3 Ck(YAE)). Single chain polypeptide TSC011 comprises from its amino to carboxyl terminus: HD37 (anti-CD19) scFv, altered human IgA2 hinge (PPPPPCPPCP, SEQ ID NO:748), human IgG1 CH2, human IgG1 CH3, and human IgG1 CH1. The nucleotide and amino acid sequences of TSC011 are set forth in SEQ ID NOS:44 and 64, respectively. Single chain polypeptide TSC004 comprises from its amino to carboxyl terminus: G19-4 (anti-CD3) scFv, altered human IgA2 hinge (SEQ ID NO:748), human IgG1 CH2, human IgG1 CH3, and human Ck(YAE). The nucleotide and amino acid sequences of TSC004 are set forth in SEQ ID NOS:37 and 57, respectively.

Bivalent polypeptide heterodimer TSC026 comprises single chain polypeptides TSC012 (HD37-“IgG1 hinge”-CH2 CH3 CH1) and TSC007 (G19-4-“IgG1 hinge”-CH2 CH3 Ck(YAE)). Single chain polypeptide TSC012 comprises from its amino to carboxyl terminus: HD37 (anti-CD19) scFv, altered human IgG1 hinge (EPKSSDKTHTSPPSPCPPCP, SEQ ID NO:750), human IgG1 CH2, human IgG1 CH3, and human IgG1 CH1. The nucleotide and amino acid sequences of TSC012 are set forth in SEQ ID NOS:45 and 65, respectively. Single chain polypeptide TSC007 comprises from its amino to carboxyl terminus: G19-4 (anti-CD3) scFv, altered human IgG1 hinge (SEQ ID NO:750), human IgG1 CH2, human IgG1 CH3, and human Ck(YAE). The nucleotide and amino acid sequences of TSC007 are set forth in SEQ ID NOS:40 and 60, respectively.

Bivalent polypeptide heterodimer TSC027 comprises single chain polypeptides TSC013 (HD37-“IgG3 hinge”-CH2 CH3 CH1) and TSC005 (G19-4-“IgG3 hinge”-CH2 CH3 Ck(YAE)). Single chain polypeptide TSC013 comprises from its amino to carboxyl terminus: HD37 (anti-CD19) scFv, altered human IgG3 hinge (EPKSSDTPPPSPRSPCPPCP, SEQ ID NO:751), human IgG1 CH2, human IgG1 CH3, and human IgG1 CH1. The nucleotide and amino acid sequences of TSC013 are set forth in SEQ ID NOS:46 and 66, respectively. Single chain polypeptide TSC005 comprises from its amino to carboxyl terminus: G19-4 (anti-CD3) scFv, altered human IgG3 hinge (SEQ ID NO:751), human IgG1 CH2, human IgG1 CH3, and human Ck(YAE). The nucleotide and amino acid sequences of TSC005 are set forth in SEQ ID NOS:38 and 58, respectively.

Bivalent polypeptide heterodimer TSC028 comprises single chain polypeptides TSC014 (HD37-“IgD hinge”-CH2 CH3 CH1) and TSC006 (G19-4-“IgD hinge”-CH2 CH3 Ck(YAE)). Single chain polypeptide TSC014 comprises from its amino to carboxyl terminus: HD37 (anti-CD19) scFv, altered human IgD hinge (ESPKAQASSVPTAQPQAEGSLAKATTAPATTRNTCPPCP, SEQ ID NO:754), human IgG1 CH2, human IgG1 CH3, and human IgG1 CH1. The nucleotide and amino acid sequences of TSC014 are set forth in SEQ ID NOS:47 and 67, respectively. Single chain polypeptide TSC006 comprises from its amino to carboxyl terminus: G19-4 (anti-CD3) scFv, altered human IgD hinge (SEQ ID NO:754), human IgG1 CH2, human IgG1 CH3, and human Ck(YAE). The nucleotide and amino acid sequences of TSC006 are set forth in SEQ ID NOS:39 and 59, respectively.

Bivalent polypeptide heterodimer TSC029 comprises single chain polypeptides TSC015 (HD37-“IgE CH2 hinge”-CH2 CH3 CH1) and TSC008 (G19-4-“IgE CH2 hinge”-CH2 CH3 Ck(YAE)). Single chain polypeptide TSC015 comprises from its amino to carboxyl terminus: HD37 (anti-CD19) scFv, altered human IgE CH2 hinge (SEQ ID NO:757), human IgG1 CH2, human IgG1 CH3, and human IgG1 CH1. The nucleotide and amino acid sequences of TSC014 are set forth in SEQ ID NOS:48 and 68, respectively. Single chain polypeptide TSC008 comprises from its amino to carboxyl terminus: G19-4 (anti-CD3) scFv, altered human IgE CH2 hinge (SEQ ID NO:757), human IgG1 CH2, human IgG1 CH3, and human Ck(YAE). The nucleotide and amino acid sequences of TSC008 are set forth in SEQ ID NOS:41 and 61, respectively.

Bivalent polypeptide heterodimer TSC030 comprises single chain polypeptides TSC016 (HD37-“IgM CH2 hinge”-CH2 CH3 CH1) and TSC009 (G19-4-“IgM CH2 hinge”-CH2 CH3 Ck(YAE)). Single chain polypeptide TSC016 comprises from its amino to carboxyl terminus: HD37 (anti-CD19) scFv, altered human IgM CH2 hinge (SEQ ID NO:759), human IgG1 CH2, human IgG1 CH3, and human IgG1 CH1. The nucleotide and amino acid sequences of TSC015 are set forth in SEQ ID NOS:49 and 69, respectively. Single chain polypeptide TSC009 comprises from its amino to carboxyl terminus: G19-4 (anti-CD3) scFv, altered human IgM CH2 hinge (SEQ ID NO:759), human IgG1 CH2, human IgG1 CH3, and human Ck(YAE). The nucleotide and amino acid sequences of TSC009 are set forth in SEQ ID NOS:42 and 62, respectively.

Bispecific polypeptide heterodimers S0268 (RON, CD3) and S0269 (CD3, c-Met) were expressed according to Example 1. The following expression levels were obtained: 2.2 μg protein/mL of culture for S0268 and 2.7 μg/mL for S0269.

### Example 5

### Cell Binding of Bispecific Polypeptide Heterodimers

To compare the effectiveness of bispecific polypeptide heterodimer molecules at targeting a tumor cell antigen and T-cells, the on-cell binding characteristics of an anti-RON (4C04 binding domain)×anti-CD3 (G19-4 binding domain) polypeptide heterodimer, S0268 (as described in Example 4), with a different bispecific scaffold (SCORPION™ protein) containing the same binding domains, S0266, were compared. Additionally, the on-cell binding characteristics of two bispecific polypeptide heterodimers, TSC020 and TSC021 (as described in Example 4), targeting different B-cell antigens (CD19 for TSC020 and CD20 for TSC021) as well as a T-cell antigen (CD3) were compared. The nucleotide and amino acid sequences of SCORPION protein S0266 are set forth in SEQ ID NOS:73 and 74, respectively. Nucleotide and amino acid sequences of the single chain polypeptides making up the bispecific polypeptide heterodimers S0268, TSC020, and TSC021 are set forth in SEQ ID NOS:26 and 52 (nucleotide), 30 and 72 (amino acid); 34 and 52 (nucleotide), 54 and 72 (amino acid); and 35 and 52 (nucleotide), 55 and 72 (amino acid), respectively (see also Example 4). Transient transfection in human 293 cells produced 6.9 μg protein/mL of culture for S0266; 2.3 μg/mL of culture for S0268; 3.0 μg/mL of culture for TSC020; and 3.2 μg/mL of culture for TSC021.

MDA-MB-453 (RON+) breast carcinoma cells, Red 1 (CD19⁺, CD20⁺) mantle cell lymphoma cells, and Jurkat (CD3⁺) T cell leukemia cells were obtained from ATCC (Manassas, Va.), and cultured according to the provided protocol. T-cells were isolated from donor PBMCs using a Pan T-cell Isolation Kit II from Miltenyi Biotec (Bergisch Gladbach, Germany). Non T-cells were separated from PBMCs by being indirectly magnetically labeled with biotin-conjugated monoclonal antibodies and anti-biotin magnetic microbeads. These cells were then depleted by retaining them in a column surrounded by a magnetic field. The T-cells were not retained in the column and were collected in the flow through.

Binding was assessed by incubating 5×10⁵ T cells or target (MDA-MB-453, Rec1, Jurkat) cells for 30 minutes at 4° C. with serially diluted bispecific molecules S0266 (αRON×αCD3 SCORPION™ protein) or S0268 (αRON×αCD3 polypeptide heterodimer) (for MDA-MB-453 cells and isolated T cells); or TSC020 (αCD19×αCD3) or TSC021 (αCD20×αCD3) for Rec1 and Jurkat cells, in concentrations from 100 nM to 0.1 nM. The cells were washed three times and then incubated with goat anti-human IgG-FITC (1:200 dilution) for another 30 minutes at 4° C. The cells were then washed again three times, fixed in 1% paraformaldehyde and read on a FACS-Calibur instrument.

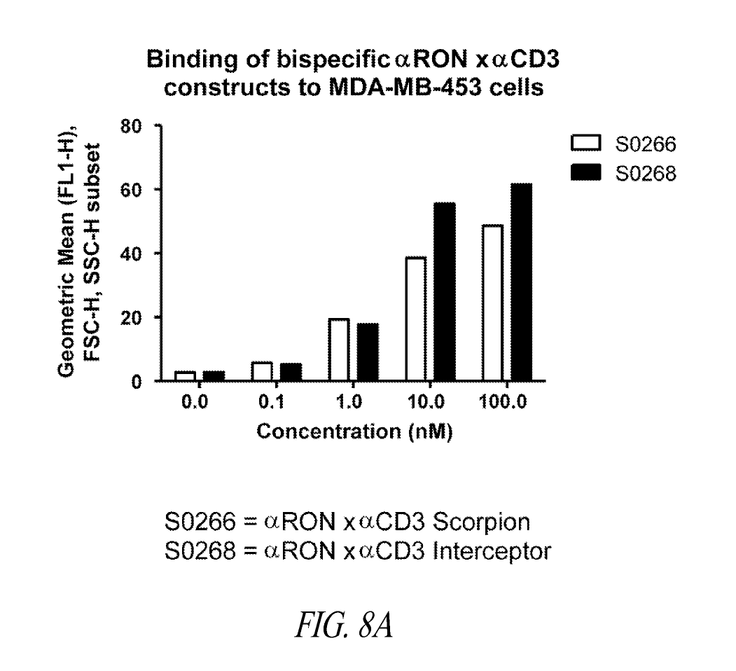

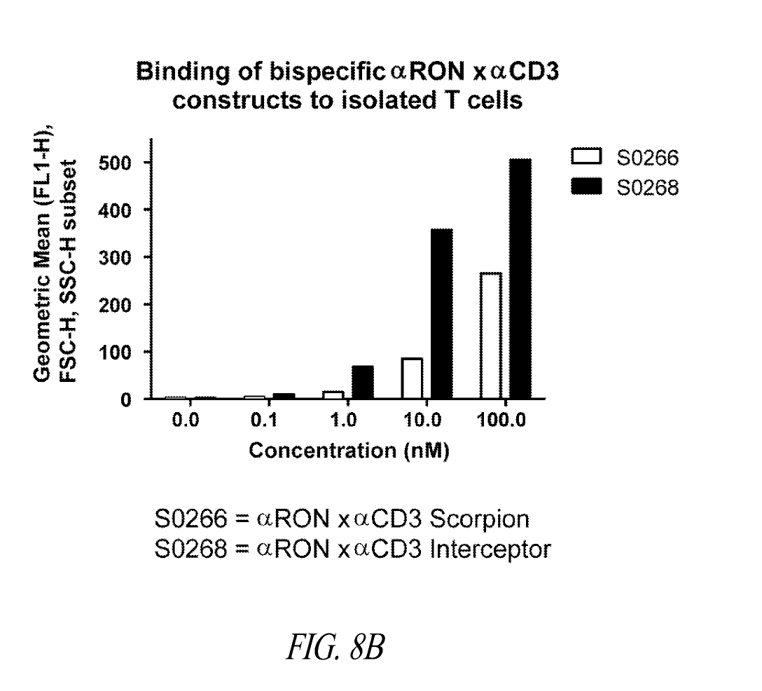

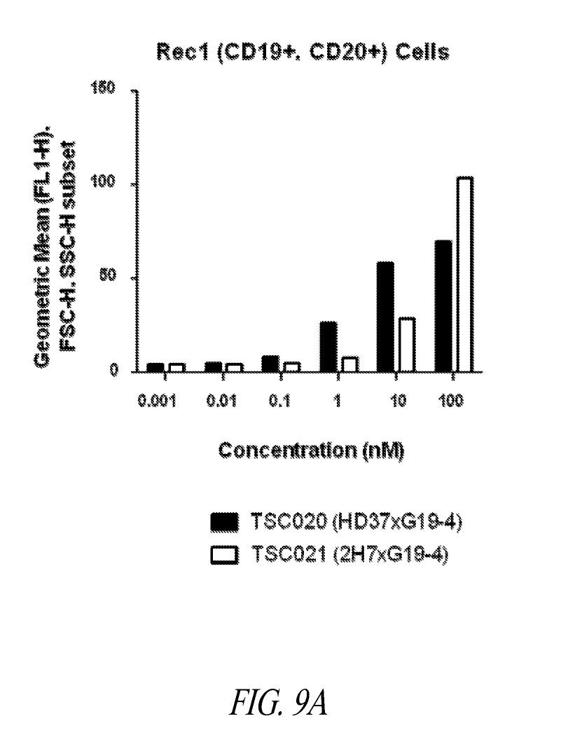

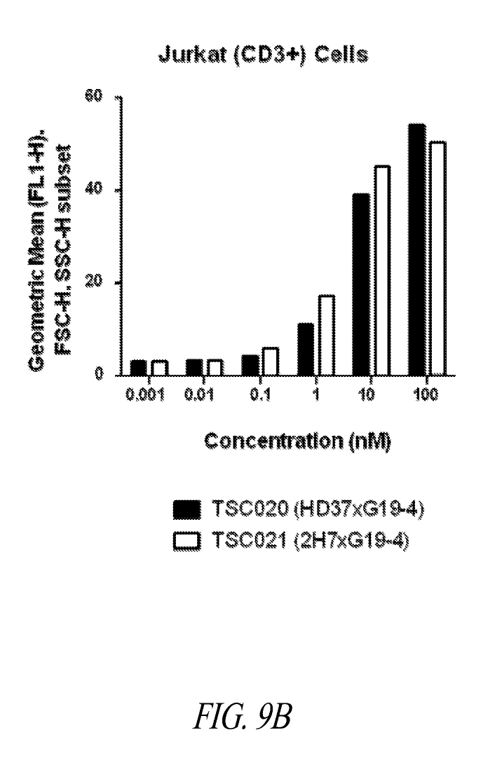

Analysis of the FSC high, SSC high subset in FlowJo v7.5 (Tree Star, Inc, Ashland, Oreg.) showed dose-dependent binding of bispecific molecules S0266 and S0268 to both MDA-MB-453 and isolated T-cells (FIGS. 8A and 8B). Unexpectedly, the S0268 polypeptide heterodimer bound with similar affinity to the comparable SCORPION™ molecule (S0266) on both MDA-MB-453 cells and T-cells, although it lacked the potential for any avidity. Higher saturation on both target cell types was also observed with the polypeptide heterodimer, which would be the case if the polypeptide heterodimer was binding at a higher stoichometry (1:1 binding of polypeptide heterodimer to surface antigen) than the equivalent SCORPION™ (potential 1:2 binding of the bivalent Scorpion to surface antigens). Comparison of the CD19-targeting bispecific heterodimer (TSC020; HD37 and G19-4 binding domains) and the CD20-targeting bispecific heterodimer (TSC021; 2H7 and G19-4 binding domains) on Rec1 and Jurkat cells revealed that the TSC020 heterodimer had higher affinity for the Rec1 cells than TSC021 heterodimer (FIG. 9A). However, both TSC020 and TSC021 heterodimers showed similar binding to CD3⁺ Jurkat cells, which is expected since both heterodimers possess the same anti-CD3 binding domain (G19-4) (FIG. 9B).

### Example 6

### Additional Bivalent & Trivalent Polypeptide Heterodimers Using Single Heterodimerization Domain Pair

Additional multispecific heterodimers were made: TSC046, TSC047, TSC048, TSC054, TSC055, TSC056, TSC057, TSC078, TSC079, TSC080, TSC099, TSC100, TSC101, and TSC102.

Bivalent polypeptide heterodimer TSC046 comprises single chain polypeptides TSC001 (HD37 CH2 CH3 CH1) and TSC039 (BMA031 CH2 CH3 Ck(YAE)). Single chain polypeptide TSC001 comprises from its amino to carboxyl terminus: HD37 (anti-CD19) scFv, human IgG1 SCC-P hinge, human IgG1 CH2, human IgG1 CH3, and human IgG1 CH1. The nucleotide and amino acid sequences of TSC001 are set forth in SEQ ID NOS:34 and 54, respectively. Single chain polypeptide TSC039 comprises from its amino to carboxyl terminus: BMA031 (anti-TCR) scFv, human IgG1 SCC-P hinge, human IgG1 CH2, human IgG1 CH3, and human Ck(YAE). The nucleotide and amino acid sequences of TSC039 are set forth in SEQ ID NOS:793 and 811, respectively.

Bivalent polypeptide heterodimer TSC047 comprises single chain polypeptides TSC001 (HD37 CH2 CH3 CH1) and TSC041 (CRIS7 CH2 CH3 Ck(YAE)). Single chain polypeptide TSC041 comprises from its amino to carboxyl terminus: CRIS7 (anti-CD3) scFv, human IgG1 SCC-P hinge, human IgG1 CH2, human IgG1 CH3, and human Ck(YAE). The nucleotide and amino acid sequences of TSC041 are set forth in SEQ ID NOS:794 and 812, respectively.

Bivalent polypeptide heterodimer TSC048 comprises single chain polypeptides TSC001 (HD37 CH2 CH3 CH1) and TSC043 (OKT3-M CH2 CH3 Ck(YAE)). Single chain polypeptide TSC043 comprises from its amino to carboxyl terminus: OKT3-M (Micromet variant anti-CD3, see also U.S. Pat. No. 7,635,472) scFv, human IgG1 SCC-P hinge, human IgG1 CH2, human IgG1 CH3, and human Ck(YAE). The nucleotide and amino acid sequences of TSC043 are set forth in SEQ ID NOs:795 and 813, respectively.

Bivalent polypeptide heterodimer TSC054 comprises single chain polypeptides TSC049 (HD37 CH2(ADCC/CDC null) CH3 CH1) and TSC053 (G19-4 CH2(ADCC/CDC null) CH3 Ck(YAE)). Single chain polypeptide TSC049 comprises from its amino to carboxyl terminus: HD37 (anti-CD19) scFv, human IgG1 SCC-P hinge, human IgG1 CH2(ADCC/CDC null) (i.e., human IgG1 CH2 with L234A, L235A, G237A, E318A, K320A, and K322A substitutions), human IgG1 CH3, and human IgG1 CH1. The nucleotide and amino acid sequences of TSC049 are set forth in SEQ ID NOS:796 and 814, respectively. Single chain polypeptide TSC053 comprises from its amino to carboxyl terminus: G19-4 (anti-CD3) scFv, human IgG1 SCC-P hinge, human IgG1 CH2(ADCC/CDC null) (i.e., human IgG1 CH2 with L234A, L235A, G237A, E318A, K320A, and K322A substitutions), human IgG1 CH3, and human Ck(YAE). The nucleotide and amino acid sequences of TSC053 are set forth in SEQ ID NOS:800 and 818, respectively.

Bivalent polypeptide heterodimer TSC055 comprises single chain polypeptides TSC050 (2H7CH2(ADCC/CDC null) CH3 CH1) and TSC053 (G19-4 CH2(ADCC/CDC null) CH3 Ck(YAE)). Single chain polypeptide TSC050 comprises from its amino to carboxyl terminus: 2H7 (anti-CD20) scFv, human IgG1 SCC-P hinge, human IgG1 CH2(ADCC/CDC null), human IgG1 CH3, and human IgG1 CH1. The nucleotide and amino acid sequences of TSC050 are set forth in SEQ ID NOS:797 and 815, respectively.

Bivalent polypeptide heterodimer TSC056 comprises single chain polypeptides TSC051 (P2C2 CH2(ADCC/CDC null) CH3 CH1) and TSC053 (G19-4 CH2(ADCC/CDC null) CH3 Ck(YAE)). Single chain polypeptide TSC051 comprises from its amino to carboxyl terminus: P2C2 (anti-CD79) scFv, human IgG1 SCC-P hinge, human IgG1 CH2(ADCC/CDC null), human IgG1 CH3, and human IgG1 CH1. The nucleotide and amino acid sequences of TSC051 are set forth in SEQ ID NOS:798 and 816, respectively.

Bivalent polypeptide heterodimer TSC057 comprises single chain polypeptides TSC052 (5D5 CH2(ADCC/CDC null) CH3 CH1) and TSC053 (G19-4 CH2(ADCC/CDC null) CH3 Ck(YAE)). Single chain polypeptide TSC052 comprises from its amino to carboxyl terminus: 5D5 (anti-cMet) scFv, human IgG1 SCC-P hinge, human IgG1 CH2(ADCC/CDC null), human IgG1 CH3, and human IgG1 CH1. The nucleotide and amino acid sequences of TSC052 are set forth in SEQ ID NOS:799 and 817, respectively.

Bivalent polypeptide heterodimer TSC078 comprises single chain polypeptides TSC049 (HD37 CH2(ADCC/CDC null) CH3 CH1) and TSC076 (OKT3 CH2(ADCC/CDC null) CH3 Ck(YAE)). Single chain polypeptide TSC076 comprises from its amino to carboxyl terminus: OKT3 (anti-CD3) scFv, human IgG1 SCC-P hinge, human IgG1 CH2(ADCC/CDC null), human IgG1 CH3, and human Ck(YAE). The nucleotide and amino acid sequences of TSC076 are set forth in SEQ ID NOS:802 and 820, respectively.

Bivalent polypeptide heterodimer TSC079 comprises single chain polypeptides TSC049 (HD37 CH2(ADCC/CDC null) CH3 CH1) and TSC077 (Nuvion CH2(ADCC/CDC null) CH3 Ck(YAE)). Single chain polypeptide TSC077 comprises from its amino to carboxyl terminus: Nuvion (anti-CD3) scFv, human IgG1 SCC-P hinge, human IgG1 CH2(ADCC/CDC null), human IgG1 CH3, and human Ck(YAE). The nucleotide and amino acid sequences of TSC077 are set forth in SEQ ID NOS:803 and 821, respectively.

Trivalent polypeptide heterodimer TSC080 comprises single chain polypeptides TSC001 (HD37 CH2 CH3 CH1) and TSC064 (G19-4 CH2 CH3 Ck(YAE) H75 Met021). Single chain polypeptide TSC064 comprises from its amino to carboxyl terminus: G19-4 (anti-CD3) scFv, human IgG1 SCC-P hinge, human IgG1 CH2, human IgG1 CH3, human Ck(YAE), H75 linker, and Met021 (anti-c-Met) scFv (with the three serine residues at the C-terminus deleted). The nucleotide and amino acid sequences of TSC064 are set forth in SEQ ID NOS:801 and 819, respectively.

Bivalent polypeptide heterodimer TSC099 comprises single chain polypeptides TSC049 (HD37 CH2(ADCC/CDC null) CH3 CH1) and TSC097 (4C04 CH2(ADCC/CDC null) CH3 Ck(YAE)). Single chain polypeptide TSC097 comprises from its amino to carboxyl terminus: 4C04 (anti-RON) scFv, human IgG1 SCC-P hinge, human IgG1 CH2(ADCC/CDC null), human IgG1 CH3, and human Ck(YAE). The nucleotide and amino acid sequences of TSC097 are set forth in SEQ ID NOS:808 and 826, respectively.

Bivalent polypeptide heterodimer TSC100 comprises single chain polypeptides TSC049 (HD37 CH2(ADCC/CDC null) CH3 CH1) and TSC093 (CRIS7 CH2(ADCC/CDC null) CH3 Ck(YAE)). Single chain polypeptide TSC093 comprises from its amino to carboxyl terminus: CRIS7 (anti-CD3) scFv, human IgG1 SCC-P hinge, human IgG1 CH2(ADCC/CDC null), human IgG1 CH3, and human Ck(YAE). The nucleotide and amino acid sequences of TSC093 are set forth in SEQ ID NOS:804 and 822, respectively.

Bivalent polypeptide heterodimer TSC101 comprises single chain polypeptides TSC049 (HD37 CH2(ADCC/CDC null) CH3 CH1) and TSC094 (OKT3-M CH2(ADCC/CDC null) CH3 Ck(YAE)). Single chain polypeptide TSC094 comprises from its amino to carboxyl terminus: OKT3-M (Micromet variant anti-CD3) scFv, human IgG1 SCC-P hinge, human IgG1 CH2(ADCC/CDC null), human IgG1 CH3, human Ck(YAE). The nucleotide and amino acid sequences of TSC094 are set forth in SEQ ID NOS:805 and 823, respectively.

Bivalent polypeptide heterodimer TSC102 comprises single chain polypeptides TSC049 (HD37 CH2(ADCC/CDC null) CH3 CH1) and TSC095 (BMA031 CH2(ADCC/CDC null) CH3 Ck(YAE)). Single chain polypeptide TSC095 comprises from its amino to carboxyl terminus: BMA031 (anti-TCR) scFv, human IgG1 SCC-P hinge, human IgG1 CH2(ADCC/CDC null), human IgG1 CH3, human Ck(YAE). The nucleotide and amino acid sequences of TSC095 are set forth in SEQ ID NOS:806 and 824, respectively.

The polypeptide heterodimer molecules were expressed and purified according to Example 1.

### Example 7

### Target-Dependent T-Cell Proliferation by Polypeptide Heterodimers

To compare the effectiveness of different bispecific polypeptide heterodimer molecules at inducing target-dependent T-cell activation and proliferation, three different bispecific molecules (TSC054, TSC078, and TSC079 as described in Example 6) with a common anti-CD19 binding domain (HD37) and three different anti-CD3 binding domains (G19-4 for TSC054, OKT3 for TSC078, HuM291 for TSC079) were compared. As a positive control, a Bispecific T-Cell Engaging molecule (BiTE) known as bsc19×3 was also prepared (see U.S. Pat. No. 7,635,472). The nucleotide and amino acid sequences for bsc19×3 are set forth in SEQ ID NOS:809 and 827, respectively. Transient transfection in human 293 cells produced 2.33 ug/mL protein for TSC054, 0.67 ug/mL protein for TSC078, and 3.5 ug/mL for TSC079.

Daudi Burkitt's lymphoma cells (CD19⁺) and MDA-MB-453 (CD19⁻) breast carcinoma cells were obtained from ATCC (Manassas, Va.) and cultured according to the provided protocol. Peripheral blood mononuclear cells (PBMC) were isolated from human blood using standard ficoll gradients. The isolated cells were washed in saline buffer. T cells were additionally isolated using a Pan T-cell Isolation Kit II from Miltenyi Biotec (Bergisch Gladbach, Germany) using the manufacturer's protocol (see also Example 5 for more information).

Proliferation was assessed by labeling isolated PBMC or T cell populations with carboxyfluorescein diacetate succinimidyl ester (CFSE). CFSE-labeled PBMC or T cells were plated in U-bottom 96-well plates at 150,000 or 100,000 cells/well, respectively, with various numbers of tumor cells, to achieve T cell to tumor cell ratios of 10:1 to 3:1. Concentrations of test molecules ranging from 8 nM to 0.08 pM were added to the cell mixtures in a total of 200 ul/well in RPMI 1640 media supplemented with 10% human or bovine serum, sodium pyruvate and non-essential amino acids. Plates were incubated at 37° C., 5% CO₂ in humidified incubators. After 3 days, cells were labeled with antibodies for flow cytometric analysis. Cells were labeled and washed in their original plates to minimize cell losses during transfers, and all labeling was done in saline buffer with 0.2% bovine serum albumin. First, cells were pre-incubated with 100 ug/ml human IgG at room temperature for 15 min. Subsequently, cells were incubated with a mixture (total volume 50 ul) of the following dye-labeled antibodies: CD5-PE, CD4-APC, CD8-Pacific Blue, CD25-PE-Cy7, as well as 7-Amino Actinomycin D (7AAD hereafter) for 40 min. Plates were washed twice, resuspended in 80 to 120 ul volumes and ran immediately in a BD LSRII flow cytometer to acquire 80% of the contents of each well. The sample files were analyzed using FlowJo software to calculate the percentages and numbers of cells that had undergone at least one cell division, according to their CFSE profile, by gating sequentially on activated, live CD4+ or CD8+ T cells (7AAD−, CD5+ CD25+ CD4+ or 7AAD− CD5+ CD25+ CD8+, respectively). Mean values and standard deviations were calculated using Microsoft Excel software. Graphs were plotted using Microsoft Excel or Graphpad Prism.

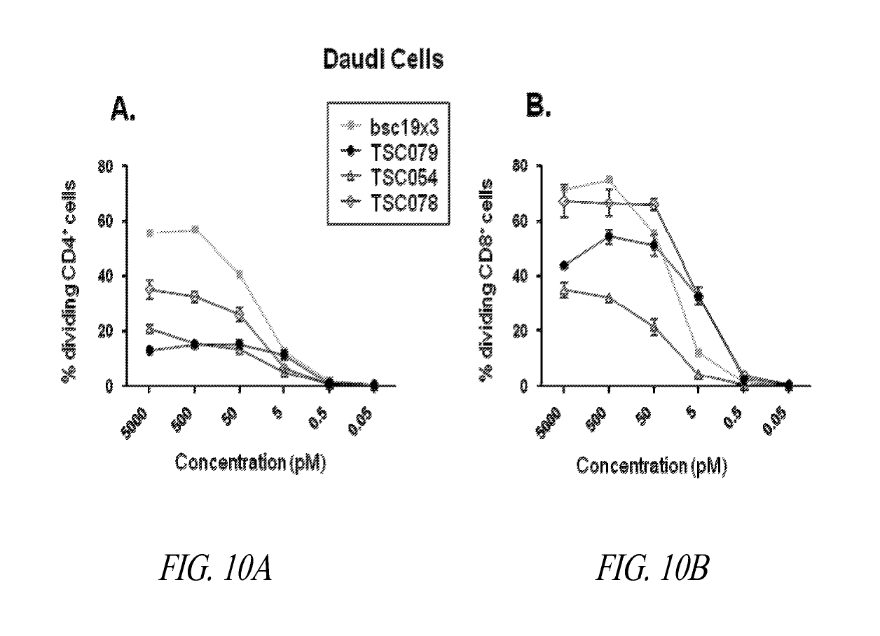

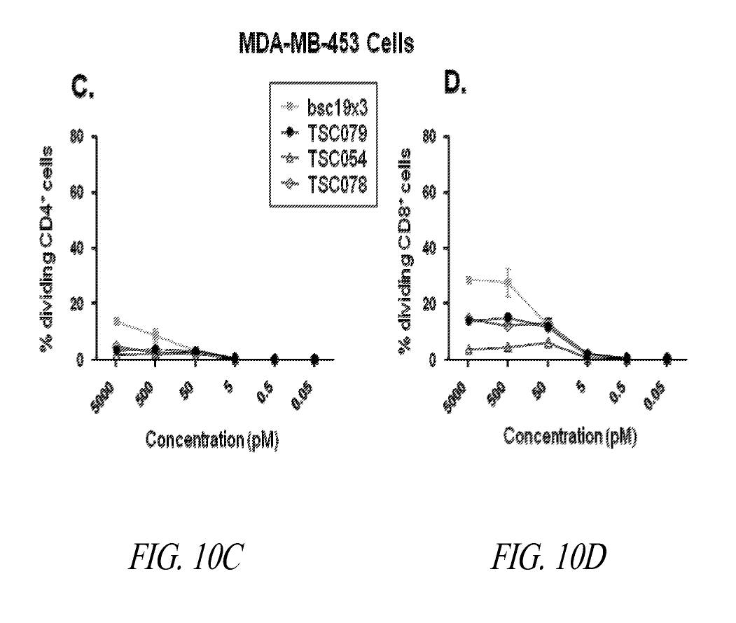

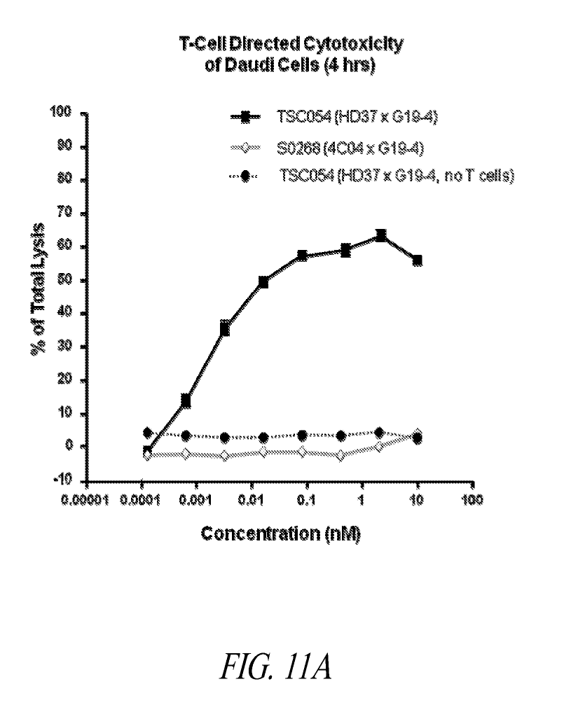

Analysis of live CD4+ and CD8+ populations from Daudi cells or MDA-MB-453 cells treated with whole PBMCs (FIGS. 10A-10D) revealed a significant increase in both the total number of cells and percent proliferating cells in the presence of Daudi cells displaying the target CD19 antigen (FIGS. 10A, 10B), but a lack of significant proliferation in the presence of MDA-MB-453 cells that lacked the CD19 antigen (FIGS. 10C, 10D). Some proliferation was observed at a lower level with the MDA-MB-453 cells and whole PBMCs, since B-cells (CD19+) were present in the PBMCs, but overall proliferation was greatly reduced in comparison. This selectivity was observed with isolated T-cells as well. Proliferation was higher for CD8+ T-cells than CD4+ T-cells in the presence of Daudi cells or MDA-MB-453 cells treated with PBMCs (FIG. 10A-10D), and the proliferation induced by TSC078 (HD37×OKT3) was generally higher at all concentrations than the response induced by TSC054 (HD37×G19-4) or TSC079 (HD37×HuM291) (FIGS. 10A-10D). Induced proliferation of CD4+ T-cells was lower in all cases for TSC054, TSC078, and TSC079 than the BiTE bsc19×3 (FIG. 10A), although TSC078 and TSC079 showed superior induction of proliferation of CD8+ cells at lower concentrations (e.g. 5 pM) than the BiTE molecule (FIG. 10B).

### Example 8

### Redirected T-Cell Cytotoxicity by Polypeptide Heterodimers

To compare the effectiveness of different bispecific polypeptide heterodimer molecules at inducing target-dependent T-cell cytotoxicity, four different bispecific molecules were compared in a chromium (⁵¹Cr) release assay. Three different bispecific molecules (TSC054, TSC078, TSC079, as described in Example 6) with a common anti-CD19 binding domain (HD37) and three different anti-CD3 binding domains (G19-4 for TSC054, OKT3 for TSC078, HuM291 for TSC079) were tested alongside a fourth bispecific molecule (S0268, described in Example 4) with an anti-RON binding domain (4C04) and an anti-CD3 binding domain (G19-4). Transient transfection in human 293 cells produced about 2.33 μg/mL protein for TSC054, about 0.67 μg/mL protein for TSC078, and about 3.5 μg/mL protein for TSC079.

Daudi Burkitt's lymphoma cells (CD19+, RON−) and BxPC-3 cells (CD19−, RON+) were obtained from ATCC (Manassas, Va.) and cultured according to the provided protocol. Peripheral blood mononuclear cells (PBMC) were isolated from human blood using standard ficoll gradients. The isolated cells were washed in saline buffer. T cells were additionally isolated using a Pan T-cell Isolation Kit II from Miltenyi Biotec (Bergisch Gladbach, Germany) using the manufacturer's protocol (see also Example 5 for more information).

Cytotoxicity was assessed by a ⁵¹Cr release assay. Approximately 5×10⁶ Daudi or BxPC-3 cells were treated with 0.3 mCi of ⁵¹Cr and incubated for 75 minutes at 37° C. After 75 minutes, cells were washed 3 times with media (RPMI+10% FBS) and resuspended in 11.5 mL of media. From this suspension, 50 μL was dispensed per well into 96 well U-bottom plates (approximately 20,000 cells/well). Concentrations of bispecific molecules ranging from 10 nM to 0.1 pM were added to the target (Daudi, BxPC-3) cells, bringing the total volume to 100 μL/well. Target cells were incubated at room temperature for 15 minutes. Then 100 μL of isolated T-cells (approximately 200,000) were added to bring the T-cell to target cell ratio to 10:1. 50 μL of 0.8% NP-40 was added to a control well containing target cells, left for 15 minutes, then 100 μL of media was added to provide a total lysis control.

Plates were incubated for 4 hours, spun at 1500 rpm for 3 minutes, and 25 μL of supernatant was transferred from each well to the corresponding well of a 96-well Luma sample plate. Sample plates were allowed to air dry in a chemical safety hood for 18 hours, and then radioactivity was read on a Topcount scintillation counter using a standard protocol.

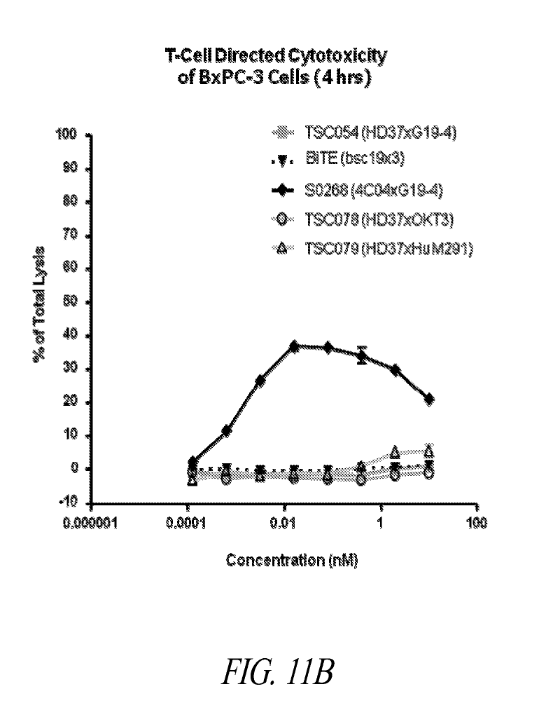

Analysis of cytotoxicity data showed a lack of off-target cytotoxicity on the Daudi (RON−) cells from the anti-RON directed bispecific molecule S0268 (FIG. 11A). Similarly, there was a lack of direct cytotoxicity observed from treating Daudi cells with TSC054 in the absence of T-cells (FIG. 11A). However, strong T-cell directed cytotoxicity was observed with the Daudi cells in the presence of T-cells and an anti-CD19 directed bispecific molecule (TSC054), reaching maximal lysis at a concentration between 10 and 100 pM (FIG. 11A). Similarly, using a second T-cell donor (FIG. 11B), no off-target cytotoxicity of the BxPC-3 (CD19−) cells was observed from the CD19-directed bispecifics TSC054, TSC078, or TSC079, or the CD19-directed BiTE bsc19×3. The anti-RON directed S0268 bispecific molecule induced cytotoxicity in BxPC-3 (RON+) cells, reaching a maximum between 10 and 100 pM (FIG. 11B).

### Example 9

### Modulation of Target-Dependent T-Cell Proliferation by Polypeptide Heterodimers with Altered Hinge Sequences

To compare the effectiveness of different bispecific polypeptide heterodimer molecules with altered hinge sequences at inducing target-dependent T-cell activation and proliferation, six different bispecific heterodimers (TSC100, TSC127, TSC165, TSC166, TSC167 and TSC168 with a common anti-CD19 binding domain (HD37), a common anti-CD3 binding domain (Cris7) and five different hinge constructs (IgG1 SCC-P hinge for TSC100 and TSC127, IgA2 hinge without the first Val residue linked to a human IgG1 core hinge for TSC165, IgG1 SSS-P hinge linked to a human IgG1 core hinge for TSC166, a portion of mutated IgG3 hinge linked to a IgG1 core hinge for TSC167, and the IgM CH2 domain without the first Val linked to a IgG1 core hinge for TSC168) were compared.

More specifically, bispecific heterodimer TSC100 is as described in Example 6.

Bispecific heterodimer TSC127 comprises single chain polypeptides TSC125 (Cris7 CH2(ADCC/CDC null) CH3 CH1) and TSC096 (HD37 CH2(ADCC/CDC null) CH3 Ck(YAE). Single chain polypeptide TSC125 comprises from its amino to carboxyl terminus: humanized Cris7 (anti-CD3) scFv, human IgG1 SCC-P hinge, human IgG1 CH2 (ADCC/CDC null), human IgG1 CH3, and human IgG1 CH1. The nucleotide and amino acid sequences of TSC125 are set forth in SEQ ID NOS:865 and 874, respectively. Single chain polypeptide TSC096 comprises from its amino to carboxyl terminus: HD37 (anti-CD19) scFv, human IgG1 SCC-P hinge, human IgG1 CH2 (ADCC/CDC null), human IgG1 CH3, and human Ck (YAE). The nucleotide and amino acid sequences of TSC096 are set forth in SEQ ID NOS:807 and 825, respectively.

Bispecific heterodimer TSC165 comprises single chain polypeptides TSC157 (Cris7 IgA2UH CH2 (ADCC/CDC null) CH3 CH1) and TSC161 (HD37 IgA2UH CH2 (ADCC/CDC null) CH3 Ck(YAE)). Single chain polypeptide TSC157 comprises from its amino to carboxyl terminus: humanized Cris7 (anti-CD3) scFv, human IgA2 hinge without the first Val linked with a human IgG1core hinge, human IgG1 CH2 (ADCC/CDC null), human IgG1 CH3, and human IgG1 CH1. The nucleotide and amino acid sequences of TSC157 are set forth in SEQ ID NOS:866 and 875, respectively. Single chain polypeptide TSC161 comprises from its amino to carboxyl terminus: HD37 (anti-CD19) scFv, human IgA2 hinge without the first Val linked with a human IgG1 core hinge, human IgG1 CH2 (ADCC/CDC null), human IgG1 CH3, and human Ck (YAE). The nucleotide and amino acid sequences of TSC161 are set forth in SEQ ID NOS:870 and 879, respectively. The amino acid sequence of the hinge used in TSC157 and TSC161 is set forth in SEQ ID NO:748.

Bispecific heterodimer TSC166 comprises single chain polypeptide TSC158 (Cris7 IgG1miniUH CH2 (ADCC/CDC null) CH3 CH1) and TSC162 (HD37 IgG1miniUH CH2 (ADCC/CDC null) CH3 Ck(YAE)). Single chain polypeptide TSC158 comprises from its amino to carboxyl terminus: humanized Cris7 (anti-CD3) scFv, human IgG1 SSC-P hinge linked with a human IgG1 core hinge, human IgG1 CH2 (ADCC/CDC null), human IgG1 CH3, and human IgG1 CH1. The nucleotide and amino acid sequences of TSC158 are set forth in SEQ ID NOS:867 and 876, respectively. Single chain polypeptide TSC162 comprises from its amino to carboxyl terminus: HD37 (anti-CD19) scFv, human IgG1 SSC-P hinge linked with a human IgG1 core hinge, human IgG1 CH2 (ADCC/CDC null), human IgG1 CH3, and human Ck (YAE). The nucleotide and amino acid sequences of TSC162 are set forth in SEQ ID NOS:871 and 880. The amino acid sequence of the hinge used in TSC158 and TSC162 is set forth in SEQ ID NO:750.

Bispecific heterodimer TSC167 comprises single chain polypeptide TSC159 (Cris7 IgG3UH CH2 (ADCC/CDC null) CH3 CH1) and TSC163 (HD37 IgG3UH CH2 (ADCC/CDC null) CH3 Ck(YAE)). Single chain polypeptide TSC159 comprises from its amino to carboxyl terminus: humanized Cris7 (anti-CD3) scFv, a portion of mutated human IgG3 hinge linked with a human IgG1 core hinge, human IgG1 CH2 (ADCC/CDC null), human IgG1 CH3, and human IgG1 CH1. The nucleotide and amino acid sequences of TSC159 are set forth in SEQ ID NOS:868 and 877, respectively. Single chain polypeptide TSC163 comprises from its amino to carboxyl terminus: HD37 (anti-CD19) scFv, a portion of mutated human IgG3 hinge linked with a human IgG1 core hinge, human IgG1 CH2 (ADCC/CDC null), human IgG1 CH3, and human Ck (YAE). The nucleotide and amino acid sequences of TSC163 are set forth in SEQ ID NOS:872 and 881. The amino acid sequence of the hinge used in TSC159 and TSC163 is set forth in SEQ ID NO:751.

Bispecific heterodimer TSC168 comprises single chain polypeptide TSC160 (Cris7 IgMCH2UH CH2 (ADCC/CDC null) CH3 CH1) and TSC164 (HD37 IgMCH2UH CH2 (ADCC/CDC null) CH3 Ck(YAE)). Single chain polypeptide TSC160 comprises from its amino to carboxyl terminus: humanized Cris7 (anti-CD3) scFv, human IgM CH2 without the first Val linked with a human IgG1 core hinge, human IgG1 CH2 (ADCC/CDC null), human IgG1 CH3, and human IgG1 CH1. The nucleotide and amino acid sequences of TSC160 are set forth in SEQ ID NOS:869 and 878, respectively. Single chain polypeptide TSC163 comprises from its amino to carboxyl terminus: HD37 (anti-CD19) scFv, human IgM CH2 without the first Val linked with a human IgG1core hinge, human IgG1 CH2 (ADCC/CDC null), human IgG1 CH3, and human Ck (YAE). The nucleotide and amino acid sequences of TSC164 are set forth in SEQ ID NOS:873 and 882. The amino acid sequence of the hinge used in TSC160 and TSC164 is set forth in SEQ ID NO:759.

As a positive control, a Bispecific T-Cell Engaging molecule (BiTE) known as bsc19×3 was also prepared (see U.S. Pat. No. 7,635,472). The nucleotide and amino acid sequences for bsc19×3 are set forth in SEQ ID NOS:809 and 827, respectively.

Transient transfection in human 293 cells produced 3.2 ug protein/mL of culture for TSC100, 6.1 ug protein/mL of culture for TSC127, 4.8 ug protein/mL of culture for TSC165, 6.2 ug protein/mL of culture for TSC166, 6.4 ug protein/mL of culture for TSC167, and 6.4 ug/mL protein for TSC168.

Daudi Burkitt's lymphoma cells (CD19⁺) and C4-2 (CD19⁻) prostate carcinoma cells were obtained from ATCC (Manassas, Va.) and MD Anderson Cancer Center (Houston, Tex.) and cultured according to the provided protocol. T cells were isolated using a Pan T-cell Isolation Kit II from Miltenyi Biotec (Bergisch Gladbach, Germany) using the manufacturer's protocol (see also Example 5 for more information).

Proliferation was assessed by labeling isolated T cell populations with carboxyfluorescein diacetate succinimidyl ester (CFSE). CFSE-labeled T cells were plated in U-bottom 96-well plates at 100,000 cells/well, with 10,000 tumor cells/well, to achieve a T cell to tumor cell ratio of 10:1. Concentrations of test molecules ranging from 5 nM to 0.005 pM were added to the cell mixtures in a total of 200 ul/well in RPMI 1640 media supplemented with 10% human or bovine serum, sodium pyruvate and non-essential amino acids. Plates were incubated at 37° C., 5% CO₂ in humidified incubators. After 3 days, cells were labeled with antibodies for flow cytometric analysis. Cells were labeled and washed in their original plates to minimize cell losses during transfers, and all labeling was done in saline buffer with 0.2% bovine serum albumin. First, cells were pre-incubated with 100 ug/ml human IgG at room temperature for 15 min. Subsequently, cells were incubated with a mixture (total volume 50 ul) of the following dye-labeled antibodies: CD5-PE, CD4-APC, CD8-Pacific Blue, CD25-PE-Cy7, as well as 7-Amino Actinomycin D (7AAD hereafter) for 40 minutes. Plates were washed twice, resuspended in 80 to 120 ul volumes and ran immediately in a BD LSRII flow cytometer to acquire 80% of the contents of each well. The sample files were analyzed using FlowJo software to calculate the percentages and numbers of cells that had undergone at least one cell division, according to their CFSE profile, by gating sequentially on activated, live CD4+ or CD8+ T cells (7AAD−, CD5+ CD25+ CD4+ or 7AAD− CD5+ CD25+ CD8+, respectively). Mean values and standard deviations were calculated using Microsoft Excel software. Graphs were plotted using Microsoft Excel or Graphpad Prism.

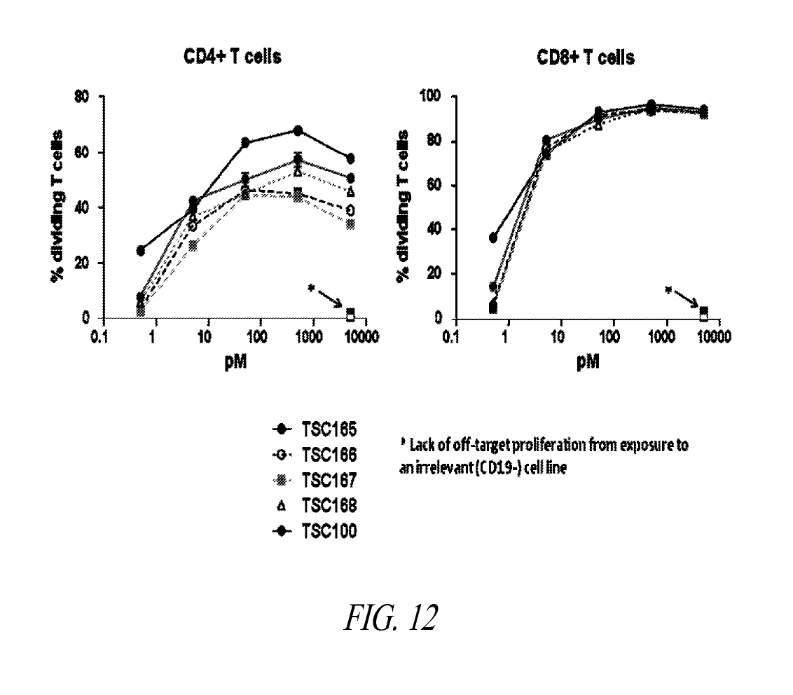

Analysis of live CD4+ and CD8+ populations from Daudi cells or C4-2 cells treated with isolated T-cells revealed a significant increase in both the total number of cells and percent proliferating cells in the presence of Daudi cells displaying the target CD19 antigen, but a lack of significant proliferation in the presence of C4-2 cells that lacked the CD19 antigen (FIG. 12). Proliferation of CD8+ cells was very similar for bispecific molecules with the default IgG1 hinge (TSC100) as well as those with longer hinges (TSC166, TSC167, TSC168). Slightly higher CD8+ proliferation at low concentrations was observed with the bispecific with the shorter IgA2 upper hinge (TSC165). Similar, but more marked differences were observed with proliferation of CD4+ cells, wherein the molecule containing the shorter IgA2 hinge (TSC165) showed higher proliferation at most concentrations than the standard IgG1 hinge (TSC100), and molecules with longer hinges (TSC166, TSC167, TSC168) showed lower proliferation.

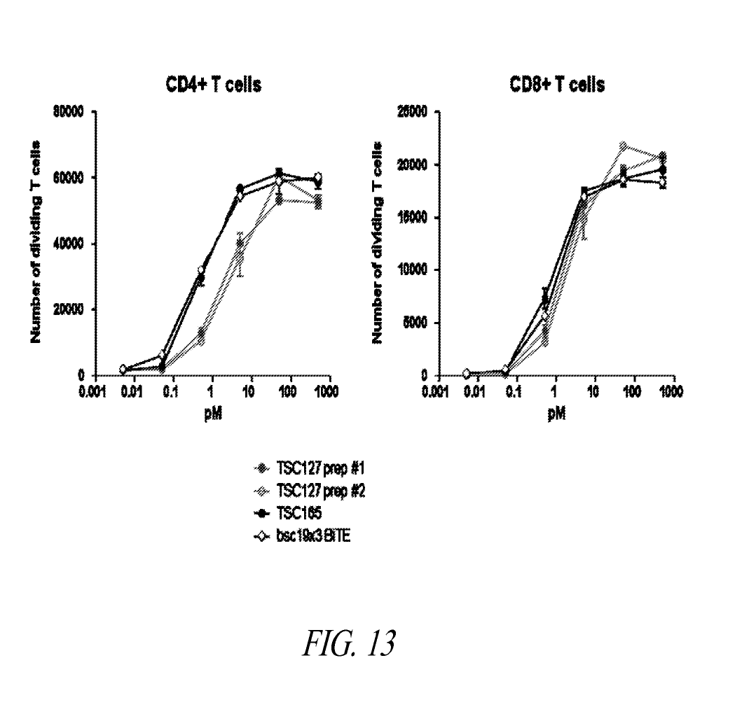

To confirm the differential activity of the IgA2 hinge, a second proliferation experiment was carried out with a titration to lower protein concentrations (FIG. 13), comparing the bispecific featuring the IgA2 hinge (TSC165) to two different production lots of a bispecific featuring the default IgG1 hinge (TSC127) and the bsc19×3 BiTE molecule. Similar to the previous experiment, less variation was observed with proliferation of CD8+ cells, with TSC165 showing comparable or greater induction of proliferation to bsc19×3, which in turn showed slightly higher or comparable proliferation to TSC127. Again, similar to the previous experiment, these trends repeated in a magnified fashion with the CD4+ cell proliferation, with TSC165 and bsc19×3 showing comparable proliferation, which in turn was noticeably greater than that of TSC127.

### Example 10

### Modulation of Redirected T-Cell Cytotoxicity by Polypeptide Heterodimers with Altered Hinge Sequences

to compare the effectiveness of changing hinge composition in bispecific polypeptide heterodimer molecules on inducing target-dependent T-cell cytotoxicity, five different bispecific molecules were compared in a chromium (⁵¹Cr) release assay. Five different bispecific molecules (TSC100, TSC165, TSC166, TSC167 and TSC168, as described in Example 9) with a common anti-CD19 binding domain (HD37), a common anti-CD3 binding domain (Cris7) and five different hinge constructs (IgG1 SCC-P hinge for TSC100 and TSC127, IgA2 hinge without the first Val linked to a human IgG1 core hinge for TSC165, IgG1 SSS-P hinge linked to a human IgG1 core hinge for TSC166, a portion of mutated IgG3 hinge linked to a human IgG1 core hinge for TSC167, and the IgM CH2 domain without the first Val linked to a human IgG1 core hinge for TSC168) were compared.

Daudi Burkitt's lymphoma cells (CD19+, RON−) were obtained from ATCC (Manassas, Va.) and cultured according to the provided protocol. T cells were isolated using a Pan T-cell Isolation Kit II from Miltenyi Biotec (Bergisch Gladbach, Germany) using the manufacturer's protocol (see also Example 5 for more information).

Cytotoxicity was assessed by a ⁵¹Cr release assay. Approximately 5×10⁶ Daudi cells were treated with 0.3 mCi of ⁵¹Cr and incubated for 75 minutes at 37° C. After 75 minutes, cells were washed 3 times with media (RPMI+10% FBS) and resuspended in 11.5 mL of media. From this suspension, 50 μL was dispensed per well into 96 well U-bottom plates (approximately 20,000 cells/well). Concentrations of bispecific molecules ranging from 10 nM to 0.1 pM were added to the target (Daudi) cells, bringing the total volume to 100 μL/well. Target cells were incubated at room temperature for 15 minutes. Then 100 μL of isolated T-cells (approximately 200,000) were added to bring the T-cell to target cell ratio to 10:1. 50 μL of 0.8% NP-40 was added to a control well containing target cells, left for 15 minutes, then 100 μL of media was added to provide a total lysis control.

Plates were incubated for 4 hours, spun at 1500 rpm for 3 minutes, and 25 μL of supernatant was transferred from each well to the corresponding well of a 96-well Luma sample plate. Sample plates were allowed to air dry in a chemical safety hood for 18 hours, and then radioactivity was read on a Topcount scintillation counter using a standard protocol.

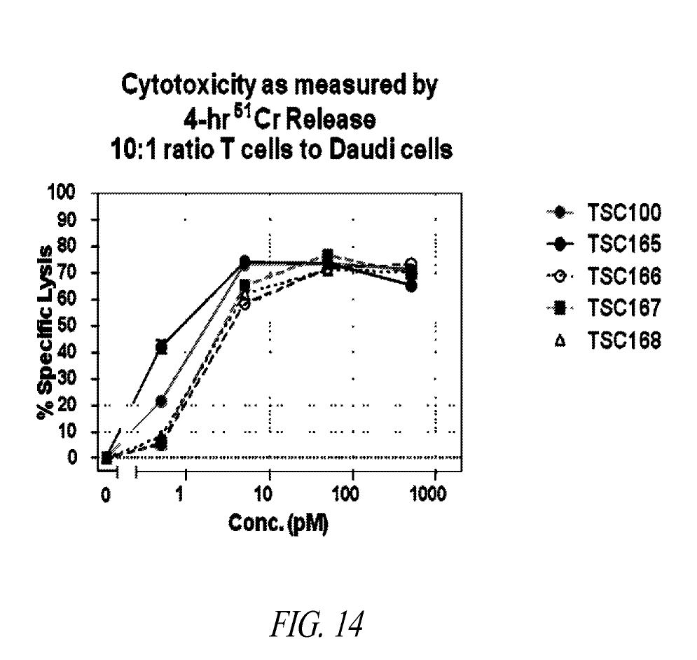

Analysis of cytotoxicity data showed strong T-cell directed cytotoxicity with the Daudi cells in the presence of T-cells and the anti-CD19 directed bispecific molecules (TSC100-TSC168), reaching maximal lysis at a concentration between 5 and 50 pM (FIG. 14). Similar to the trends observed in Example 9, the bispecific molecule with a shorter IgA2 upper hinge region (TSC165) showed comparable or greater cytotoxicity to the molecule with the standard IgG1 upper hinge region (TSC100), whereas the molecules with longer upper hinge regions (TSC166, TSC167, TSC168) were less potent at inducing cytotoxicity.

### Example 11

### Bispecific Heterodimers with CH3-CH1 and CH3-Ck Linker Variations

The following bispecific heterodimers with CH3-CH1 and CH3-Ck linker variations were made:

Bispecific heterodimer TSC151 comprises single chain polypeptides TSC145 and TSC148. Single chain polypeptide TSC145 comprises from its amino to carboxyl terminus: humanized Cris7 (anti-CD3) scFv, human IgG1 SCC-P hinge, human CH2 (ADCC/CDC null), human CH3, and human IgG1 CH1. The human CH3 and human IgG1 CH1 are linked by a linker having the sequence GGGSS (SEQ ID NO:847). The nucleotide and amino acid sequences of TSC145 are set forth in SEQ ID NOS:853 and 859, respectively. Single chain polypeptide TSC148 comprises from its amino to carboxyl terminus: HD37 (anti-CD19) scFv, human IgG1 SCC-P hinge, human CH2 (ADCC/CDC null), human CH3, and human Ck (YAE). The human CH3 and human Ck (YAE) are linked with a linker having the sequence GGGSR (SEQ ID NO:850) in which R may alternatively be regarded as the first arginine of human Ck (YAE). The nucleotide and amino acid sequences of TSC148 are set forth in SEQ ID NOS:856 and 862, respectively.

Bispecific heterodimer TSC152 comprises single chain polypeptides TSC146 and TSC149. Single chain polypeptide TSC146 comprises from its amino to carboxyl terminus: humanized Cris7 (anti-CD3) scFv, human IgG1 SCC-P hinge, human CH2 (ADCC/CDC null), human CH3, and human IgG1 CH1. The human CH3 and human IgG1 CH1 are linked by a linker having the sequence SYSPNS (SEQ ID NO:848). The nucleotide and amino acid sequences of TSC146 are set forth in SEQ ID NOS:854 and 860, respectively. Single chain polypeptide TSC149 comprises from its amino to carboxyl terminus: HD37 (anti-CD19) scFv, human IgG1 SCC-P hinge, human CH2 (ADCC/CDC null), human CH3, and human Ck (YAE). The human CH3 and human Ck (YAE) are linked with a linker having the sequence SYSPNSR (SEQ ID NO:851) in which R may alternatively be regarded as the first arginine of human Ck (YAE). The nucleotide and amino acid sequences of TSC149 are set forth in SEQ ID NOS:857 and 863, respectively.

Bispecific heterodimer TSC153 comprises single chain polypeptides TSC147 and TSC150. Single chain polypeptide TSC147 comprises from its amino to carboxyl terminus: humanized Cris7 (anti-CD3) scFv, human IgG1 SCC-P hinge, human CH2 (ADCC/CDC null), human CH3, and human IgG1 CH1. The human CH3 and human IgG1 CH1 are linked by a linker having the sequence SSLNTPNS (SEQ ID NO:849). The nucleotide and amino acid sequences of TSC147 are set forth in SEQ ID NOS:855 and 861, respectively. Single chain polypeptide TSC150 comprises from its amino to carboxyl terminus: HD37 (anti-CD19) scFv, human IgG1 SCC-P hinge, human CH2 (ADCC/CDC null), human CH3, and human Ck (YAE). The human CH3 and human Ck (YAE) are linked with a linker having the sequence SSLNTPNSR (SEQ ID NO:852) in which R may alternatively be regarded as the first arginine of human Ck (YAE). The nucleotide and amino acid sequences of TSC150 are set forth in SEQ ID NOS:858 and 864, respectively.

The above bispecific heterodimers were expressed according to Example 1. The following expression levels were obtained: 9.2 μg protein/ml of culture for TSC151, 11.2 μg protein/ml of culture for TSC152, and 14.7 μg protein/ml of culture for TSC153. In comparison, about 6 μg protein/ml of culture was obtained for heterodimers with a CH3-CH1 and CH3-Ck linker having the amino acid sequence KSR (SEQ ID NO:846).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A polypeptide heterodimer, comprising:
(a) a first single chain polypeptide (SCP-I) comprising from one to four binding domains that specifically bind from one to four targets, a hinge (H-I), an immunoglobulin heterodimerization domain (HD-I), and an Fc region portion (FRP-I); and
(b) a second single chain polypeptide (SCP-II) comprising from zero to four binding domains that specifically bind from zero to four targets, a hinge (H-II), an immunoglobulin heterodimerization domain (HD-II), and an Fc region portion (FRP-II);
wherein
(i) the immunoglobulin heterodimerization domain of the first single chain polypeptide (HD-I) and the immunoglobulin heterodimerization domain of the second single chain polypeptide (HD-II) preferentially associate with each other to form a polypeptide heterodimer comprised of the first single chain polypeptide (SCP-I) and the second single chain polypeptide (SCP-II), and
(1) the immunoglobulin heterodimerization domain of the first single chain polypeptide (HD-I) comprises a first immunoglobulin CHI region, and the immunoglobulin heterodimerization domain of the second single chain polypeptide (HD-II) comprises a first immunoglobulin CL region, or
(2) the immunoglobulin heterodimerization domain of the first single chain polypeptide (HD-I) comprises a first immunoglobulin CL region, and the immunoglobulin heterodimerization domain of the second single chain polypeptide (HD-II) comprises a first immunoglobulin CHI region; and
(ii) the Fc region portion of the first single chain polypeptide (FCP-I) and the Fc region portion of the second single chain polypeptide (FCP-II) each comprise an immunoglobulin CH2 and CH3 domain of IgG1, IgG2, IgG3, IgG4, IgA1, IgA2, IgD, or any combination thereof; an immunoglobulin CH3 domain of IgG1, IgG2, IgG3, IgG4, IgA1, IgA2, IgD, IgE, IgM, or any combination thereof; or an immunoglobulin CH3 and CH4 domain of IgE, IgM, or a combination thereof,
provided that the polypeptide heterodimer comprises at least two binding domains that specifically bind at least two different targets.

2. The polypeptide heterodimer of claim 1, wherein the binding domains are single chain Fv (scFv) polypeptides.

3. The polypeptide heterodimer of claim 1, wherein the polypeptide heterodimer comprises two binding domains (BD1 and BD2), three binding domains (BD1, BD2 and BD3), four binding domains (BD1, BD2, BD3 and BD4), or five to eight binding domains.

4-15. (canceled)

16. The polypeptide heterodimer of claim 1, wherein at least one of the binding domains is an agonist of IL-10, HLA-G, HGF, IL-35, PD-1, BTLA, TNFR1, TNFR2, DR4, DR5, TWEAKR, or FAS.

17. The polypeptide heterodimer of claim 1, wherein at least one binding domain specifically binds a TCR complex or a component thereof, and at least another binding domain specifically binds to PSMA, CD79b, CD19, HLA-DR, CD20, RON, c-Met, or CEACAM-6.

18. The polypeptide heterodimer of claim 1, wherein at least one binding domain specifically binds to CD28, and at least another binding domain specifically binds to or is an antagonist of, CD79b, hyperIL-6, PDL2, monoIL-10, CD86, LIGHT, GITRL, CD40, PDL1, HVEM, or LTBR.

19. The polypeptide heterodimer of claim 1, wherein at least one binding domain specifically binds to CD28, and at least another binding domain is an agonist of IL-10, HLA-G, HGF, IL-35, PD-1, or BTLA.

20. The polypeptide heterodimer of claim 1, wherein the immunoglobulin heterodimerization domain of the first single chain polypeptide (HD-I) comprises the first immunoglobulin CHI region and the immunoglobulin heterodimerization domain of the second single chain polypeptide (HD-II) comprises the first immunoglobulin CL region.

21. (canceled)

22. (canceled)

23. The polypeptide heterodimer of claim 20 wherein the first single chain polypeptide further comprises a second CHI region and the second single chain polypeptide further comprises a second CL region, and wherein the second CHI region of the first single chain polypeptide and the second CL region of the second single chain polypeptide associate with each other in the polypeptide heterodimer.

24-26. (canceled)

27. The polypeptide heterodimer of claim 1, wherein the immunoglobulin heterodimerization domain of the first single chain polypeptide (HD-I) comprises a first immunoglobulin CL region, and the immunoglobulin heterodimerization domain of the second single chain polypeptide (HD-II) comprises a first immunoglobulin CHI region.

28. (canceled)

29. (canceled)

30. The polypeptide heterodimer of claim 27 wherein the first single chain polypeptide further comprises a second CL region and the second single chain polypeptide further comprises a second CHI region, and wherein the second CL region of the first single chain polypeptide and the second CHI region of the second single chain polypeptide associate with each other in the polypeptide heterodimer.

31-40. (canceled)

41. The polypeptide heterodimer of claim 1, wherein the first CL region is a Cκ region or a Cλ region.

42. (canceled)

43. The polypeptide heterodimer of claim 23, wherein the second CL region is a Cκ region or a Cλ region.

44. (canceled)

45. The polypeptide heterodimer of claim 41, wherein the Cκ region is a wild type human immunoglobulin Cκ region.

46. The polypeptide heterodimer of claim 41, wherein the Cκ region is an altered human immunoglobulin Cκ region with one or more amino acids of a wild type human Cκ region substituted at N29, N30, Q52, V55, T56, S68, or T70.

47. (canceled)

48. The polypeptide heterodimer of claim 41, wherein the CHI region is an altered human immunoglobulin CHI region comprising an amino acid substitution by which Val (V) at position 68 is substituted by Lys (K), Arg (R) or His (H), and wherein the Cκ region is an altered human immunoglobulin Cκ region comprising an amino acid substitution by which Leu (L) at position 27 is substituted by Asp (D) or Glu (E).

49. The polypeptide heterodimer of claim 41, wherein the CHI region is an altered human immunoglobulin CHI region comprising an amino acid substitution by which Val (V) at position 68 is changed to Asp (D) or Glu (E), and wherein the Cκ region is an altered human immunoglobulin Cκ region comprising an amino acid substitution by which Leu (L) at position 27 is changed to Lys (K), Arg (R) or His (H).

50-52. (canceled)

53. The polypeptide heterodimer of claim 1, wherein the first CHI region is an altered human immunoglobulin CHI region with the cysteine of a wild type human immunoglobulin CHI region that is involved in forming a disulfide bond with a wild type human immunoglobulin CL region deleted or substituted.

54-56. (canceled)

57. The polypeptide heterodimer of claim 53, wherein the first CHI region is a polypeptide comprising SEQ ID NO: 114, 844 or 845.

58. The polypeptide heterodimer of claim 41, wherein the Cκ region is selected from any one of the polypeptides comprising SEQ IL NOS: 141-178, 202, and 838-843.

59. The polypeptide heterodimer of claim 41, wherein the Cλ region is a polypeptide comprising SEQ ID NO: 140.

60. The polypeptide heterodimer of claim 1, wherein the Fc region portion of the first single chain polypeptide (FRP-I) and the Fc region portion of the second single chain polypeptide (FRP-II) each comprise an immunoglobulin CH2 domain of an immunoglobulin CH3 domain.

61-65. (canceled)

66. The polypeptide heterodimer of claim 1, wherein the Fc region portion of the first single chain polypeptide (FRP-I) and the Fc region portion of the second single chain polypeptide (FRP-II) each comprise an immunoglobulin CH2 domain and an immunoglobulin CH3 domain.

67-74. (canceled)

75. The polypeptide heterodimer of claim 1, wherein the hinge of both the first and second single chain polypeptides is an immunoglobulin hinge region.

76-78. (canceled)

79. The polypeptide heterodimer of claim 75, wherein the hinge region is
(a) amino terminal to the Fc region portion,
(b) disposed between the binding domain and the immunoglobulin heterodimerization domain,
(c) disposed between the immunoglobulin heterodimerization domain and the Fc region portion, or
(d) at the amino terminus of the first or second single chain polypeptide.

80-83. (canceled)

84. The polypeptide heterodimer of claim 1, wherein the hinges of the first and second single chain polypeptides are different.

85-87. (canceled)

88. The heterodimer of claim 1, wherein the first and second single chain polypeptides comprise SEQ ID NOS: SEQ ID NOS:2 and 4, SEQ ID NOS:6 and 8, SEQ ID NOS: 10 and 12, SEQ ID NOS: 14 and 16, SEQ ID NOS: 18 and 20, SEQ ID NOS:20 and 22, SEQ ID NOS:20 and 24, SEQ ID NOS:30 and 32, SEQ ID NOS:29 and 31, SEQ ID NOS:29 and 32, SEQ ID NOS:30 and 72, SEQ ID NOS:53 and 72, SEQ ID NOS:54 and 72, SEQ ID NOS:55 and 72, SEQ ID NOS:70 and 72, SEQ ID NOS:71 and 72, SEQ ID NOS:63 and 56, SEQ ID NOS:64 and 57, SEQ ID NOS:65 and 60, SEQ ID NOS:66 and 58, SEQ ID NOS:67 and 59, SEQ ID NOS:68 and 61, SEQ ID NOS:69 and 62, SEQ ID NOS:54 and 811, SEQ ID NOS:54 and 812, SEQ ID NOS:54 and 813, SEQ ID NOS:814 and 818, SEQ ID NOS:815 and 818, SEQ ID NOS:816 and 818, SEQ ID NOS:817 and 818, SEQ ID NOS:814 and 820, SEQ ID NOS:814 and 821, SEQ ID NOS:54 and 819, SEQ ID NOS:814 and 826, SEQ ID NOS:814 and 822, SEQ ID NOS: 814 and 823, SEQ ID NOS:814 and 824, SEQ ID NOS:859 and 862, SEQ ID NOS:860 and 863, SEQ ID NOS:861 and 864, SEQ ID NOS:874 and 825, SEQ ID NOS:875 and 879, SEQ ID NOS:876 and 880, SEQ ID NO:877 and 881, or SEQ ID NOS:878 and 882.

89. A composition comprising a polypeptide heterodimer of claim 1 and a pharmaceutically acceptable excipient.

90. An expression vector capable of expressing the polypeptide heterodimer of claim 1, comprising a first polynucleotide encoding the first single chain polypeptide and a second polynucleotide encoding the second single chain polypeptide.

91. A host cell comprising the expression vector of claim 90.

92. A host cell comprising first and second expression vectors capable of expressing the first and second single chain polypeptides, respectively, of the polypeptide heterodimer of claim 1.

93. A method for making a polypeptide heterodimer, comprising
(a) culturing the host cell of claim 91 under conditions suitable to express first and second single chain polypeptides, and
(b) optionally isolating or purifying the heterodimers formed from the first and second single chain polypeptides from the culture.

94. A method for directing T cell activation, comprising administering to a patient in need thereof an effective amount of a polypeptide heterodimer according to claim 1, wherein the polypeptide heterodimer comprises a binding domain that specifically binds TCRα, TCRβ, CD3γ, CD3ε, CD3β or a combination thereof, and a second binding domain that specifically binds a different target.

95. A method for inhibiting growth, metastasis or metastatic growth of a malignancy, comprising administering to a patient in need thereof an effective amount of a polypeptide heterodimer according to claim 1, wherein the polypeptide heterodimer comprises a binding domain that specifically binds TCRα, TCRβ, CD3γ, CD3ε, CD3ε, c-Met, or Ron.

96. A method for treating an autoimmune or inflammatory condition, comprising administering to a patient in need thereof an effective amount of a polypeptide heterodimer according to claim 1, wherein the polypeptide heterodimer comprises a binding domain that specifically binds TCRα, TCRβ, CD3γ, CD3ε, CDR or CD28.

97. A method for treating a B-cell associated disorder or disease comprising administering to a patient in need thereof an effective amount of a polypeptide heterodimer according to claim 1, wherein the polypeptide heterodimer comprises a binding domain that specifically binds TCRα, TCRβ, CD3γ, CD3ε, CD3β, and a second binding domain that specifically binds to CD19, CD20, CD79b or HLA-DR.

98-101. (canceled)
